(19) United States
(12) Patent Application Publication
(10) Pub. No.: US 2022/0218047 A1
(43) Pub. Date: Jul. 14, 2022

(54) PNEUMATIC TRAINING DEVICE AND GARMENT FOR INCREASING STRENGTH

(71) Applicant: Sean Tremaine Whalen, Mountain View, CA (US)

(72) Inventor: Sean Tremaine Whalen, Mountain View, CA (US)

(21) Appl. No.: 17/656,930

(22) Filed: Mar. 29, 2022

Related U.S. Application Data
(63) Continuation of application No. 16/034,730, filed on Jul. 13, 2018.
(60) Provisional application No. 62/533,008, filed on Jul. 15, 2017.

(51) Int. Cl. A41D 13/00; A61B 17/132; A63B 21/00; A61B 5/00; A61B 5/022; A61B 17/135; A63B 23/035; A63B 69/00; A41D 1/00; A63B 21/008; A63B 24/00

(52) U.S. Cl. CPC A41D 13/0015; A61B 5/024; A63B 21/4009; A61B 5/6804; A63B 21/4025; A61B 5/022; A63B 21/4011; A61B 17/135; A63B 23/03508; A63B 21/4017; A63B 69/0057; A41D 1/002; A63B 21/0085; A63B 24/0087; A61B 17/1327; A61B 17/1325

(57) ABSTRACT

A device for performing blood flow restriction training during the day, integrated with a garment, and controllable to apply a desired compression level to a range of muscles with the intent on improving the health and fitness of a user doing normal daily activities.

### CROSS-REFERENCE TO RELATED APPLICATIONS

This application is a continuation of U.S. Ser. No. 16/034,730 filed on Jul. 13, 2018 and entitled “Pneumatic Training Device and Garment for Increasing Strength.” U.S. Ser. No. 16/034,730 claims priority to and the benefit of U.S. Provisional Ser. No. 62/533,008 filed on Jul. 15, 2017 and entitled “Pneumatic Blood Flow Restriction Training Garment and Method.” by Whalen. Each of the foregoing applications are hereby incorporated by reference in their entirety, but except for any subject matter disclaimers or disavowals, and except to the extent in conflict with the express disclosure of this document, in which case the language of this document shall control.

### FIELD

This invention relates to blood flow restriction systems, and more specifically to a garment designed for standalone use or in conjunction with an inflatable belt design for use therein, to provide a simple to use convenient way to integrate BFR into a daily lifestyle.

### BACKGROUND

The muscle training apparatus, system, and method described in prior art, and herein in this application is spreading fast globally because of its beneficial effects as described below. In addition, national and foreign physicians as well as universities have conducted blood flow restriction research investigations, as a result of them, researchers have published many articles.

The muscle strength increasing method according to these patents is a distinctive non-conventional one that involves compression of an arm or leg at a position near the top thereof. This muscle strength increasing method (the subject muscle strength increasing method is herein referred to as a “Blood flow restriction muscle training method” or simply BFR).

Muscles are composed of slow-twitch muscle fibers and fast-twitch muscle fibers. Slow-twitch muscle fibers are limited in their potential for growth. Accordingly, it is necessary to the recruit fast-twitch muscle fibers in the muscle in order to develop the muscles. Recruitment of fast-twitch muscle fibers causes lactic acid buildup in the muscles, which triggers secretion of growth hormone from the pituitary. The growth hormone has effects of, for example, promoting muscle growth and shedding body fat. This means that recruitment and exhaustion of fast-twitch muscle fibers results in development of fast-twitch muscle fibers and, in turn, the entire muscle.

Slow-twitch muscle fibers and fast-twitch muscle fibers are different from each other in terms of the following. Slow-twitch muscle fibers use oxygen for energy and are recruited for low-intensity endurance activities. Fast-twitch muscle fibers provide for activities regardless of whether or not oxygen is present. They are recruited after the slow-twitch muscle fibers for highly intense activities. Therefore, it is necessary to cause the earlier recruited and activated slow-twitch muscle fibers to be exhausted soon in order to recruit fast-twitch muscle fibers.

Conventional muscle strength increasing methods use heavy load with, for example, a barbell to cause the slow-twitch muscle fibers to be exhausted first, and then to recruit the fast-twitch muscle fibers. This recruitment of fast-twitch muscle fibers requires a significant amount of force generation from the muscle, is time-consuming, and tends to increase the burden on muscles and joints.

On the other hand, muscle exercise may be performed under the restriction of muscle blood flow into the limb distal to a predetermined position by means of applying pressure upon the muscles at the predetermined position near the top of the limb. Since less oxygen is supplied to these muscles, the slow-twitch muscle fibers, which require oxygen for energy, are thus exhausted in a short period of time. Muscle exercises with blood-flow restriction by application of pressure will result in recruitment of the fast-twitch muscle fibers without needing a large amount of exercises. More specifically, when pressure is applied circumferentially upon a limb at a predetermined position near the top of the limb, venous circulation is restricted while arterial circulation is kept almost the same as the normal condition if an appropriate pressure is applied, This is because veins are closer to the skin surface of the limb, and are thinner and less muscular (less resistant against an force for pressurization) than arteries while arteries are found deep within the limb, and are thicker and more muscular than veins. By holding that condition for a certain period of time, the limb that has compressed near the top thereof becomes engorged with blood which runs from arteries but cannot flow through veins. This promotes a state of blood pooling in the capillaries where such an amount of blood is not flowing normally. The limb that is compressed at a position near the top thereof gets into a state as if it were doing heavy exercises. During this time, because of the temporal occlusion of the veins, the muscle fatigue is caused by the fact that the lactic acid that has built up in the muscles is less likely to be removed from the muscles. Furthermore, the brain receives information of strenuous exercise from muscles, and brain's physiological action is then responsible for the production of much more growth hormone than is usually produced during the daily life for muscle regeneration as well as during typical exercises.

In other words, BFR training contributes to artificially produce a state which otherwise will occur during and after heavy exercises. It is possible to cause muscle fatigue much more heavily than would be produced normally with that amount of exercises. In addition, the user can “trick” the brain into secreting a larger amount of growth hormone.

Because of the aforementioned mechanism, restriction of muscle blood flow can allow users to significantly develop their muscles.

As the applicant will describe, prior art in the form of patents and product for sale by the applicant and other inventors describe a means of applying an external apparatus, namely a belt/band/strap, either inflatable or non-inflatable to the user's body for performing BFR training. While this method and equipment is convenient in some settings, i.e. going to a gym or a sports practice, it may be cumbersome for casual users who are not accustomed to carrying equipment or taking time out of their day to work out. Further, it has been shown in research that adding BFR during normal daily activities such as walking can improve functional outcomes. Therefore, a system, or preferably a garment that is configured to perform BFR training throughout a day doing daily activities may provide casual users benefits they would otherwise not have the motivation or habit to do the work to achieve. The reader shall note that the application may refer to a blood flow restriction training garment, an integrated garment, or simply a garment and the terms shall be equivalent for the purposes of this application unless otherwise specified. A person for example who is not accustomed to consciously doing an exercise routine during the day is unlikely to utilize the prior art because it takes investment in equipment, but more importantly a change to behavior which is difficult to effect. The applicant's invention as will be described herein allows a casual user who does not normally exercise, as well as those who do, to get the benefits of BFR training without doing anything different than they normally do throughout the day. Therefore the applicant's invention is a much more practical system to adopt than external BFR systems like the prior art that require setting aside fixed points in time to do the training and special gear that must carried around and strapped on in order to do so.

One of the important education factors with BFR training is placement, both location and orientation, and initial tension of the belts as discussed in prior art. By locating and orienting the belts on a garment that is to be worn by the user, the belts are automatically located for the user and removes this educational requirement and potential for mistakes that would make the training either less effective or potentially dangerous.

Another aspect of current systems that hinders use is the fact that additional equipment must be carried and different clothing used in order to perform exercise. Then the belts must be placed over the clothing and a setup process takes some amount of time. The applicant has seen how in practice the everyday lives of most people is too busy to consciously stop and put on special gear and designate a certain time for doing exercise. Therefore the applicant's invention of integrated garments and further augmentation with automated sensory triggers and methods removes the user's need to actually do anything other than put their clothes on in the morning, which is something everyone has to do anyway.

Additionally, for busy professionals, or anyone for that matter, putting external belts or bands over clothing can a) wrinkle the clothing and b) draws attention that might cause embarrassment. The applicant's inventions alleviate both these issues as they can be worn as undergarments and easily activated during the day, or even automatically activated during the day without drawing attention to the user. In contrast to prior art, the applicant's integration of compression means and conventional clothing form the thinnest possible system to be inconspicuous, vs. prior art products, particularly pneumatic BFR products, developed by the applicant, by KAATSU and by other companies that are bulky and must be worn external to clothing.

### Compression Garments

There are many examples in the prior art of compression garments designed to push fluid out of an extremity and promote circulation and reduce edema in a limb. Products such as the Game Ready, Recovery Pump or Normatec boots are specifically designed in order to do this and are meant for use post exercise for recovery to reduce edema. Non-pneumatic mechanisms such as compression stockings and other garments similarly have the same purpose, to push fluid OUT of the extremity and prevent pooling which can cause DVTs etc.

This field or category of product and construction has a completely different and opposite purpose than the applicant's invention for BFR training. As described above and in prior applications to the applicant, the goal of BFR is actually to trap fluid and blood in the extremity, and NOT let it escape except briefly during muscle contractions, but in all cases to reduce overall blood flow, not increase it. By doing this, the metabolites accumulate and you get the disturbance of homeostasis necessary to properly perform BFR. In the case of compression garments, pneumatic or not, the blood or fluid being forced out of the limb actually works against the purpose of BFR and is an aid to circulation, NOT an impediment. Their intended purposes is not to build muscle but to assist in circulation. Therefore, the reader shall note that there are stark differences in implementation and these types of compression garment prior art are designed and invented to produce exactly the OPPOSITE physiological manipulation to the applicant's invention.

### BFR Shirts and Shorts

Lowery proposed the concept of a BFR garment wrap (https://prezi.com/kwliuyls_dkl/practical-blood-flow-restriction-garment-wraps/) wherein elastic or non-stretch straps would be part of a pair of compression shorts or shirts for creating an instant wrap for occluding blood flow. Lowery however does not elaborate on any details around how this would be effectively achieved and further does not contemplate any pneumatic garment system for doing BFR training.

A simple wrap or non-inflatable strap is impractical for such an application for several reasons. For starters, if the garment is loose fitting (Lowery only contemplates tight fitting garments), as many undergarments generally are, there is no tension or ability to easily cinch up the strap on the upper body where only one arm is available. Further, in a loose fitting garment, even on the legs, fabric would need to bunch up significantly underneath the wrap and this would position the wrap away from the surface and make it difficult to apply adequate compression, and make it uncomfortable for the user to have bunch up fabric squished against their skin.

Further, a strap must be manually tensioned and loosened each time to apply and remove restriction which takes an action from the user, and requires access to the wrap under normal clothing. This makes application during the day difficult because one needs to get underneath shirts or pants for example to access a wrap vs. the applicant's pneumatic concepts that may be inflated via external access.

Further, the wrap or non-inflatable strap is very difficult to apply appropriately with even tension and surface compression around the circumference. It is easily over tensioned as well leading to an unsafe condition in the muscle that can lead to occlusion and subsequent health complications. Finally, to be effective, wraps must be wrapped more than one time around the limb in order to adequately restrict blood flow to be effective and it is difficult to do this when you have the wrap under a shirt or pair of work pants for example. Inherently a wrap is not a very good system for doing BFR training because it is not repeatable, it is not controllable, it is inaccurate, and it is cumbersome to apply.

Additionally, in the applicant's invention, the compression means may be worn under clothing and accessing the strapping mechanisms suggested in Lowery requires removing external clothing, such as work clothes for example, in order to access the straps. This is neither appropriate nor practical for a working professional during the day for example and the applicant's invention solves this fundamental problem with the prior art. Neither is it appropriate in an athletic training session, for example American football. If worn during training, the strap would be covered by padding and inaccessible and require removal of padding and gear which is impractical. The prior art further does not elaborate on various usage mechanisms or sensing and protocols that can be combined with “garment” based BFR to achieve results and improvements in quality of life for casual users who are not necessarily accustomed or pre-disposed to do exercise routines. A pull strap wrap as Lowery describes is not automated as well, and therefore lacks the benefits of the applicant's invention which easily and discretely is adjusted to adequately and effectively restrict flow in the limb, potentially without needing intervention from the user. The applicant could not find prior art or patents which address the concept of an combining compression means with clothing that is designed for user every day in general living conditions. While Lowery has proposed a garment with a non-inflatable strap, such mechanisms have not been accepted by the market. The applicant contends that this is because simply providing of a strap connected to a shirt or shorts does not alleviate the fundamental problem in that strapping is not an adequate or user-friendly way of restricting blood flow to achieve results and it is cumbersome and still requires significant education and experience by the user.

### Discrete BFR Mechanism

While other prior art addresses discrete belts, bands, or cuffs for performing BFR training, all of these mechanisms, including the applicant's, are designed for external use over clothing and as a temporary condition during a training period, not as a “casual wear” system. As such, it is unlikely that many users will utilize such systems on a regular basis if they are not already accustomed to doing so. This leaves out a large segment of the population (the sedentary or infirm in particular) from enjoying the benefits of BFR training. Behavioral changes are extremely difficult and the applicant's invention addresses this fundamental challenge that the user need not alter anything they are doing yet may still reap the rewards of BFR training. Additionally the applicant's inventions pave the way for usage methods, such as worker health management, that have not even been contemplated in prior art and are not practical without a system like the applicant proposes for receiving data about and controlling multiple BFR garments remotely.

### BACKGROUND OF THE INVENTION—OBJECTS AND ADVANTAGES

Accordingly, besides the objects and advantages of a garment for use in a blood flow restriction system described in this specification, several objects and advantages of the present invention are:
a) To provide a BFR system that is easy to use in regular daily life
b) To provide a BFR system that is inexpensive and can be used and washed and cleaned every day as a regular wear garment.
c) To provide a method in which a user may automatically take advantage of the benefits of BFR without altering their daily activities.
d) To provide a method for monitoring health and safety of a BFR user.
e) To provide a system for monitoring and controlling multiple BFR garments.
f) To provide a system that is easy to mass produce.
g) To provide a system that is imperceptible to an outside observe when it is worn by a user.
h) To provide a system that includes replaceable compression means.
i) To provide a system that includes integral compression means.
j) To provide a connectable system for remote monitoring and control.
k) The provide a BFR garment that comfortably accommodates users of different body types and sizes.
l) To provide a BFR garment that minimizes pressure spikes during muscle contractions.
m) To provide a BFR garment that can comfortably be worn, activated, and de-activated without significant action from the user.

Still further objects and advantages will become apparent from a consideration of the ensuing description and drawings.

### SUMMARY

In accordance with the present invention, a garment is provided with optional attachment means for securing a belt in order to apply a compression around a range of muscles to be compressed, and the garment may be worn as a standalone garment or may be concealed under an external piece of clothing.

### BRIEF DESCRIPTION OF THE DRAWINGS

FIG. 1A—shows a BFR garment as a shirt with air pathways and/or electrical conduits on a front face of the garment with connection means at both ends for transporting air pressure and/or electrical signals from an attachment area for a compression means to the chest area.

FIG. 1B—shows a BFR garment as a shirt with air pathways running along an out-seam with connection means at both ends for transporting air pressure from near an attachment area for a compression means to a second point near the hip or waist area.

FIG. 1C—shows a BFR garment as a shirt with air pathways with connection means at both ends for transporting air pressure or electrical signals from near an attachment area for a compression means to a second point located near the back or waist area where the air pathway runs along a back face of the shirt, and one air pathway is removable from the shirt.

FIG. 2A—shows a BFR garment as a pair of shorts with air pathways and/or electrical conduits running along an out-seam, with connection means at one end for transporting air and/or electrical signals from an integrally formed compression means in the form of a pneumatic belt located around the groin region to a second point located in the hip or waist region of the user.

FIG. 2B—shows a BFR garment similar to FIG. 2A wherein two air pathways are joined together at an air pathway junction to form a single reservoir and only one connection means is needed to inflate both compression means.

FIG. 3A—shows a BFR garment in the form of a shirt with a liner comprising the BFR components of FIG. 1A where the liner and BFR components are hidden from public view by the convention clothing sleeve of the shirt, and the liner comprises both an integrated compression means and an attachment means for attaching a separable compression means.

FIG. 3B—shows a BFR garment in the form of a skirt with a liner comprising BFR components of FIG. 2A wherein the liner and BFR components are hidden from public view by the skirt, and the BFR components are integrally formed in the BFR garment, and the BFR components may be used to shape the upper leg or buttocks region of the user.

FIG. 4—shows a BFR garment in the form of tight fitting pants with attachment means for securing a control system near the ankle in the form of a pocket, air pathways and/or electrical conduits with connection means at both ends for transporting pressurized air and/or electrical signals to the legs and arms, and attachment means for attaching a compression means to each leg near the groin region.

FIG. 5—shows two BFR garments in the form of convention clothing items: shirt and shorts for use together as a full body system, where shirt and shorts have integrated compression means at the upper arms and upper thigh respectively, each has separable air pathways with connection means on one end for plugging into a control system comprising at least an inflatable mechanism, and attachment means in the form of a belt for clipping in the control system around the waist area of the user.

FIG. 6—shows a BFR garment in the form of a shirt with integrated compression means on each upper arm and an attachment means for attaching a control system in the form of a removable necklace worn by the user, and the control system is in communication with air pathways and/or electrical conduits suitable for transporting pressurized gas and/or electrical signals between the compression means and the control system.

FIG. 7A—shows an attachment means for connecting a compression means to a BFR garment comprising a series of button snaps distributed circumferentially around a portion of the garment and mating snaps disposed on one side of the compression means, depicted as a pneumatic belt.

FIG. 7B—shows an attachment means for securing a compression means to a BFR garment comprising a flap with a attachment means shown as a series of button snaps, and the flap may form a sleeve to enclose a portion of the compression means, shown as an inflatable belt, when the flap's connection means is secured, and the compression means is substantially held in place for tensioning.

FIG. 7C—shows an attachment means for connecting a compression means to a BFR garment comprising a sleeve connected to the garment through which the compression means may be fed to secure the compression means to the garment in a pre-determined location.

FIG. 7D—shows an attachment means for connecting a compression means to a BFR garment comprising a series of loops connected to the garment through which the compression means may be fed to secure the compression means to the garment in a pre-determined location.

FIG. 7E—shows an attachment means for connecting a compression means to a BFR garment comprising a series of periodically placed, shown as loop fasteners, to mate with a matching attachment means on the compression means shown as hook fasteners, the periodic spacing allowing an elastic conventional clothing element to stretch for accommodating varying limb sizes.

FIG. 8A—shows an attachment means in the form of button snaps for attaching a compression means to a BFR garment, further depicting an initial tensioning means configurable to provide a pre-determined compression level to the user prior to locking down the compression means.

FIG. 8B—shows the system of FIG. 8A without the initial tensioning means and further shows a marking system for locking the compression means to the garment at a pre-determined location and apply a desired initial tension.

FIG. 8C—shows an initial tensioning means comprising a pair of flexible members in the form of draw strings with an adjustable stop and slide lock on each draw string, and a portion of fabric integrally formed with the garment, where tensioning of the draw strings until the adjustable stop is reached reduces the circumference of a section of the garment over a range of muscles to be compressed.

FIG. 8D—shows a variation on the initial tensioning means of FIG. 8C whereby the draw string mechanism is disposed on the compression means itself, and the belt is attached to the garment via attachment means, and the draw string mechanism is movable and adjustable along the outer surface of the belt to adjust for different limb girths.

FIG. 8E—shows a section view of one example of a loose fitting garment illustrating the adjustment process of the BFR garment to accommodate limbs of different girths whereby a non-connected portion of the BFR garment is movable separate from an connected portion of the compression means, and the non-connected portion of the BFR garment may bunch up underneath the compression means as the compression means is adjusted to apply sufficient initial tension and locate the compression means in the open position.

FIG. 8F—shows a leg section of a BFR garment comprising multiple cutout profiles around the circumference for weaving in and attaching a compression means, and the cutout profiles secure the position of the compression means relative to the user and allow the BFR garment to stretch to accommodate users of different limb sizes.

FIG. 8G—shows a cross section view of FIG. 8F illustrating how the compression means may weave in and out of the cutout profiles and attach to the BFR garment with fastening means depicted as sections of hook and loop fastener.

FIG. 9A—shows a control system comprising basic and optional components in order to actuate and control a compressive force on the range of muscles to be compressed.

FIG. 9B—shows a system comprising an external controller and multiple BFR garments worn by one or more users and the external controller is used to gather data about the users and their sessions from the BFR garments and control compression levels and programming of the BFR garments.

FIG. 10A—shows a method of programming and automatically controlling a BFR garment without intervention from a user to apply a safe and effective compression level to the range of muscles to be compressed.

FIG. 10B—shows a method of controlling multiple BFR garments via an external controller.

FIG. 11—shows a method of remotely monitoring the health and safety of a BFR user wearing a BFR garment.

FIG. 12A—shows a section view of a compression means integrated into a BFR garment during a manufacturing process such that the user cannot remove the compression means after usage, comprising an elastic expandable bladder with an adjustable non-stretch outer barrier for limiting outward expansion of the bladder.

FIG. 12B—shows a section view of a compression means integrated into a BFR garment similar to FIG. 12A, the bladder and garment being elastic and stretchable, and a portion of the bladder is covered with a non-stretch outer barrier layer to limit expansion of that particular section of the bladder.

FIG. 13A—shows another style of integrated compression means in an unprepared state and BFR garment with a fold up flap style outer sheath to secure an outer circumference of an elastic bladder with draw strings and lock the outer circumference in the open position.

FIG. 13B—shows the integrated compression garment of FIG. 13A in the prepared position where the fold up flap covers the inflatable chambers and outer bladder layer to prevent it from expanding under pressure

FIG. 14A—shows a flat sample pattern for an integrated BFR garment in the form of one leg of a pair of shorts using substantially airtight material wherein a bladder is integrally formed at a point along the height of the shorts leg by adding a single non-stretch layer along the outer surface in a sufficiently airtight manner and the shorts material comprises the airtight inner bladder layer.

FIG. 14B—shows a variation on the sample pattern of FIG. 14A wherein a separate bladder component comprised of substantially non-stretch material in the form of a barrel inflatable belt is attached to a bottom layer of the shorts around a portion of the perimeter of one leg, and an optional webbing is disposed under a portion of the bladder.

FIG. 15A—shows a BFR garment as a pair of shorts with an integrated compression means that is non-stretch disposed around a portion of the circumference and the shorts have a slit on the end that allows a portion of the shorts to expand to accommodate legs of different sizes.

FIG. 15B—shows a BFR garment similar to 15A wherein the slit is located along the length and the cutout profile allows the expansion of the shorts without producing undue stress on the fabric which would reduce durability.

FIG. 16A—shows a BFR garment where the compression means is covering less than the full circumference and connected to the clothing element along the full length of the compression means, and the stretch in the clothing element in the bladder end gap is sufficient to accommodate a range of larger limb sizes and may additionally fold back and overlap to accommodate a range of smaller limb sizes, and a series of fastening means, shown as hook and loop fasteners secure an adjustable fastening means to look in an open position.

FIG. 16B—shows one example of how to integrally form a compression means using the conventional clothing element and an outer layer with either stitching or bonding.

FIG. 16C—shows another version of FIG. 16B with an added stiffener to force inflation of the compression means radially inward on a limb.

FIG. 16D—shows another version of FIG. 16C wherein an outer barrier layer is integrated with the BFR garment to limit outward expansion of the compression means.

### DRAWINGS—REFERENCE NUMERALS

1A-K—device
100—BFR garment
101—inner belt material
102—outer belt material
103—Inflatable chamber
104—input port
105—compression means
106—inflation means
107—connection means
108—conventional clothing element
109—
110—first fastening means
111—second fastening means
112—stitch line
113—
114—attachment means
115—liner
116—sleeve
117—pocket
118—out-seam
119—air pathway
120—inseam
121—front face
122—back face
123—air pathway junction
124—reservoir
125—electrical conduit
126—
127—loop
128—first point
129—second point
130—
131—
132—fabric loop
133—neck strap
134—belt
700—button snap
701—fold up flap
800—initial tension means
801—marking guide
802—elastic member
803—slide lock
804—adjustable stop
805—adjustable fastening means
806—first end
807—second end
808—opening
809—connected portion
810—non-connected portion
811—open position
812—connection point
813—anchor point
814—strap
815—flexible member
900—control system
901—memory storage means
902—processing means
903—
904—communication means
905—energy storage means
906—sensing means
907—external controller
908—data
909—external reservoir
1200—
1201—inner bladder layer
1202—outer bladder layer
1203—outer barrier layer
1204—bladder connection joint
1205—fold line
1500—slit garment
1501—first slit edge
1502—second slit edge
1503—webbing
1504—cutout profile
1601—seam tape
1602—stiffener
1603—bladder end gap

### DETAILED DESCRIPTION

### Preferred Embodiment—Description

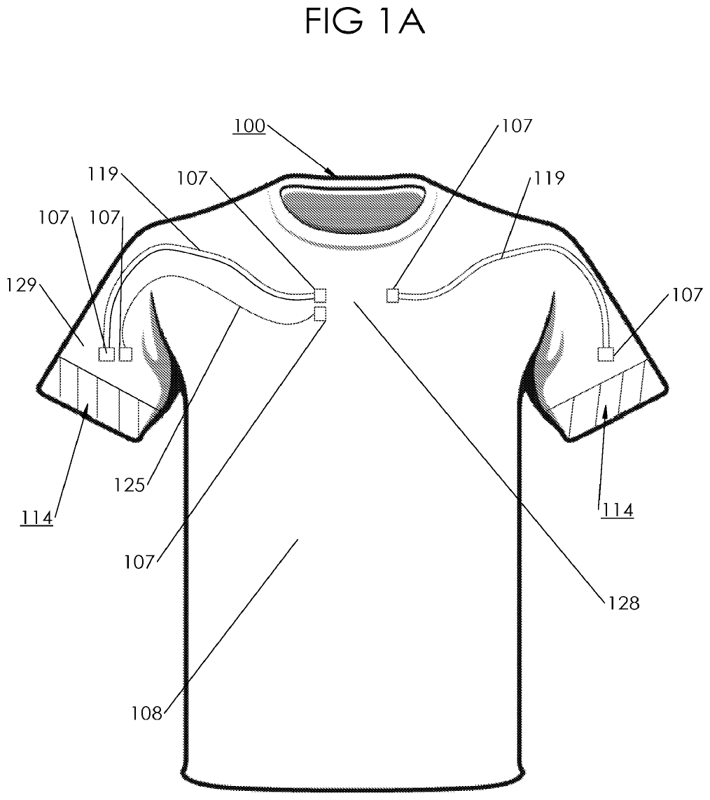

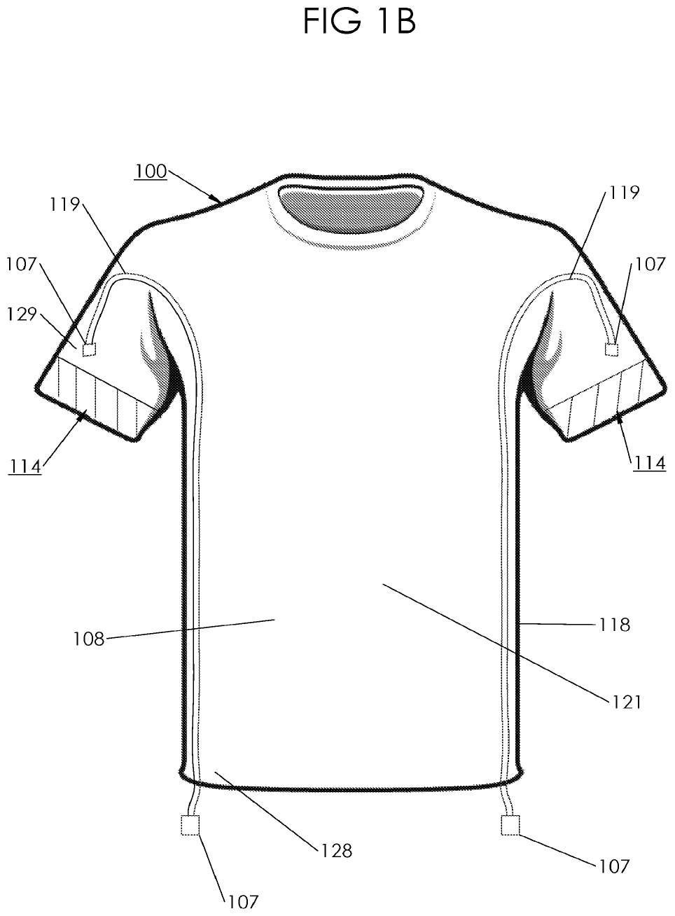

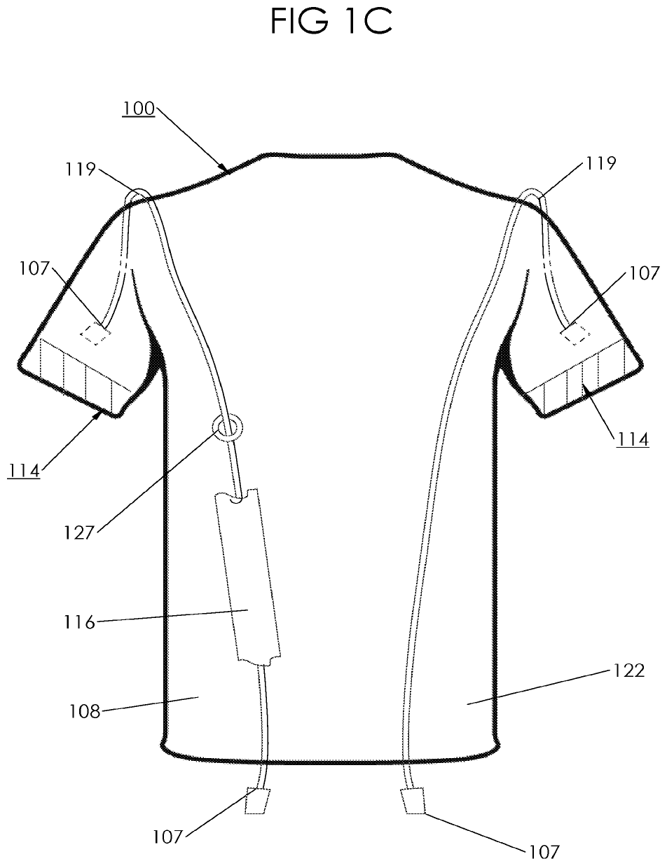

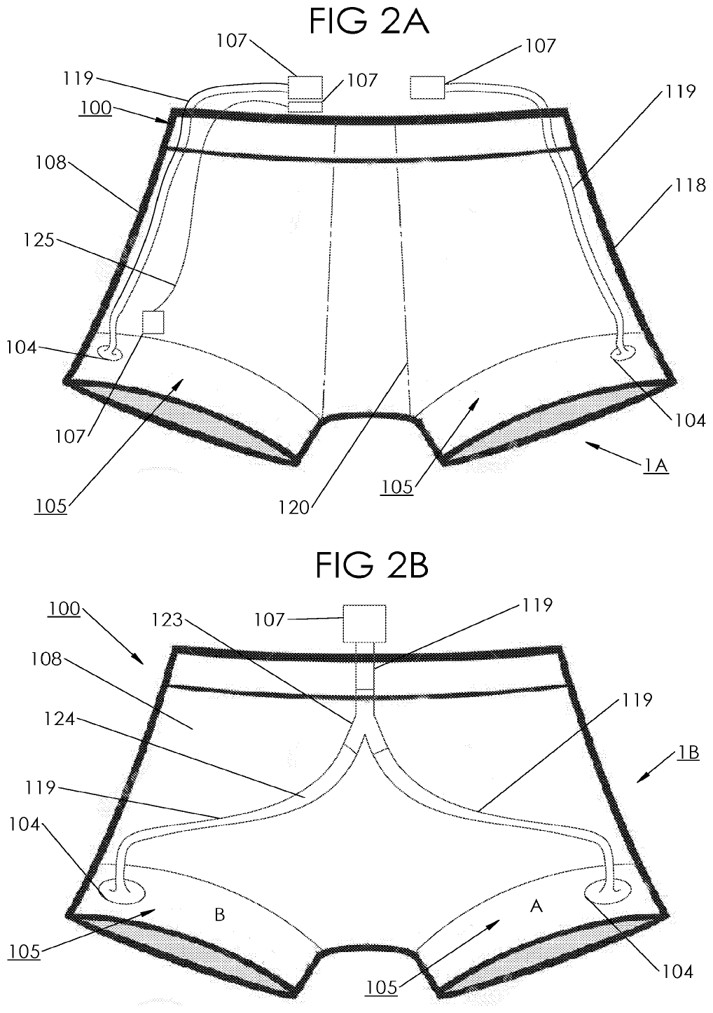

A device 1 for blood flow restriction training is described herein and shown in various figs such as FIG. 2A (device 1A), 2B (device 1B), 3 (device 1C), 3B (device 1D), 5 (device 1E), 6 (device 1F), 9B (device 1G and 1H), 15A (device 11), 15B (device 1J), 16A (device 1K). The device 1, device with reference numeral 1 is the general concept vs. specific implementations shown in the FIGS. 1A-K, may be an independent device 1 or attached to the garment. The device 1 may be removably attached to the garment 100 or integrated together with a garment. It is understood that the devices 1,1A-K are independently configurable and interchangeable with all other devices 1,1A-K herein, in that each may be independent, attachable to a garment 100 (otherwise a component of the garment 100 referred to as a conventional clothing element 108 and explained later), or attached and formed integrally with the garment 100 itself whether explicitly stated as such or not. The device 1 may comprise additional features and functions in whole or in part, such as, but not limited to: compression means 105, air pathway 119, connection means 107, attachment means 114, initial tension means 800, adjustable fastening means 805, external controller 907s, system controllers with option components of inflation means 106, sensing means 906, processing means 902, etc. The reader shall understand therefore that the device 1 is a broad combination of various elements and elements may be added or removed as deemed necessary to cover the particular embodiments described here, or further inferred from the applicants specification. Furthermore, each of the attachment means 114 or methods described herein for attaching the device 1 to the garment 100 may be used with any of the other embodiments of the device 1.

A preferred embodiment of the device 1 K, as attached to a BFR garment 100, alternatively referred to as simply a garment 100 in this specification and claims, for use in a BFR system is shown in 16A and described below. As stated the device 1 may comprise additional functions and features beyond what is shown in a given figure, and otherwise described or inferred herein.

### Compression Means—

The foundational component of the device 1 is a compression means 105 and the description below references the compression means 105 attached to, removable from, or integrated with a garment. The reader shall understand in these the garment 100 is a separate entity and not part of the device 1. Compression means 105 have been extensively covered in the prior art to both the applicant and other inventors. One feature of the applicant's invention disclosed herein is that the concepts are adaptable to many of the prior disclosed concepts for compression means 105 including both pneumatic and non-pneumatic designs such as an elastic wrap or tension strap as Sato has described. For the sake of brevity, the descriptions herein will focus on pneumatic means as these are more controllable, practical, safe, and viable overall than tension straps as has been described in previous applications and was disclosed by Lowery.

Compression means 105 therefore preferably comprise an inflatable bladder made of suitable substantially airtight materials. The materials have been extensively covered previously, but for review may be a polyurethane or PVC laminate, for example 0.080″ thick, welded together on a perimeter, and the laminate may be backed with a nylon material, for example 200 denier. An inner belt material 101 may be either the polyurethane film or the film laminated onto a substrate fabric like a nylon or a Lycra fabric and may be non-stretch or elastic. Likewise the outer belt material 102 may also be of the same construction options. As described in relation to various embodiments, the conventional clothing element 108 may be adapted to be substantially airtight and may form either the inner bladder layer 1201 or outer bladder layer 1202, thereby reducing components, cost, and bulk as in Alternate Embodiments which describe how to integrate the device 1 and compression means 105 with the garment.

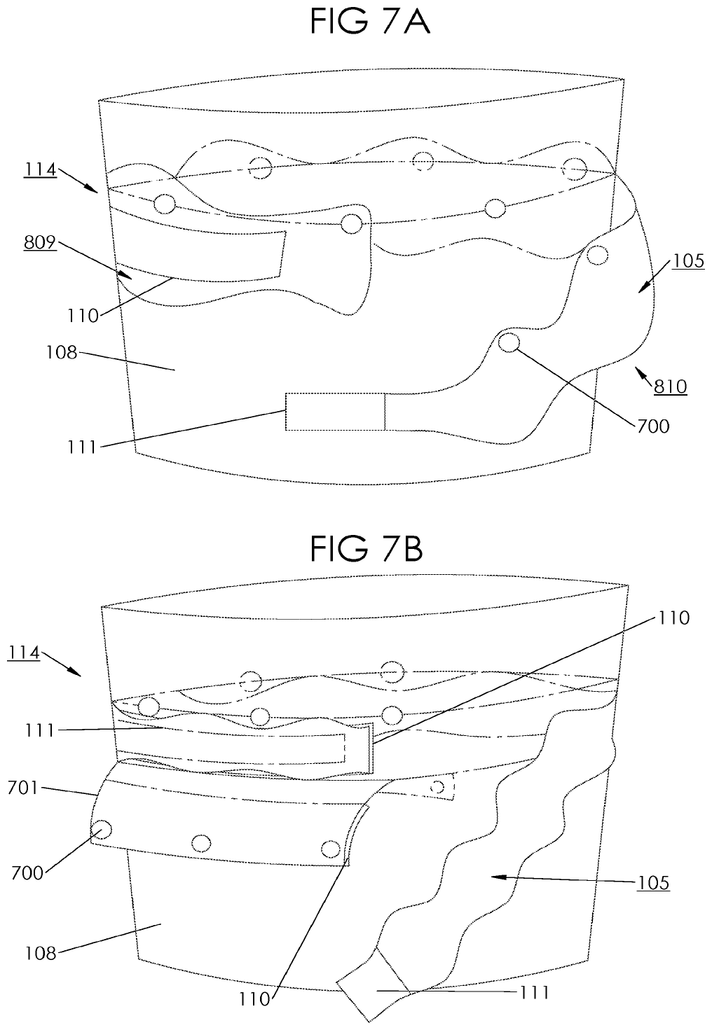

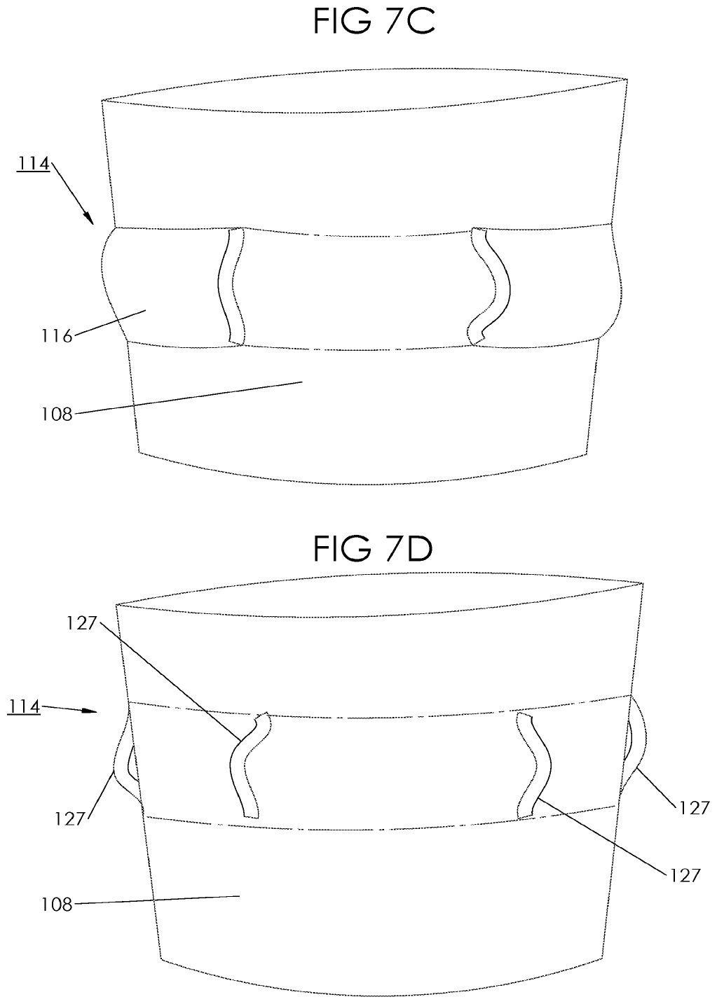

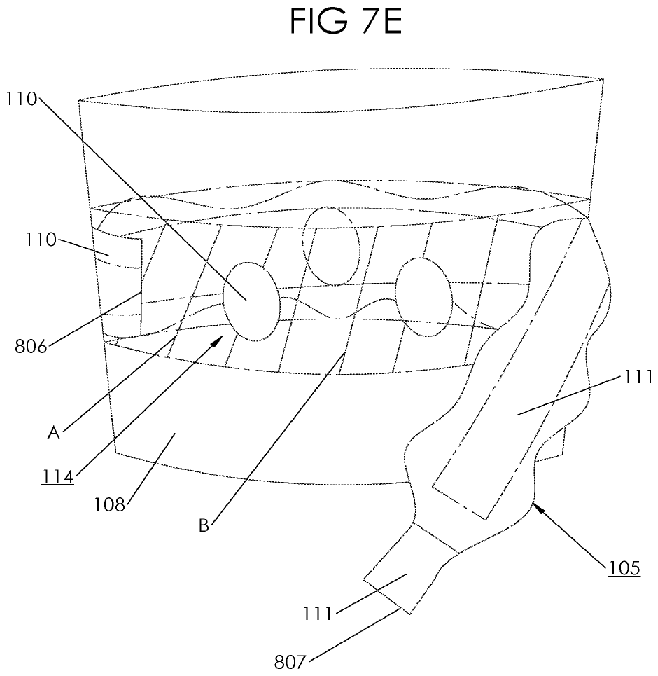

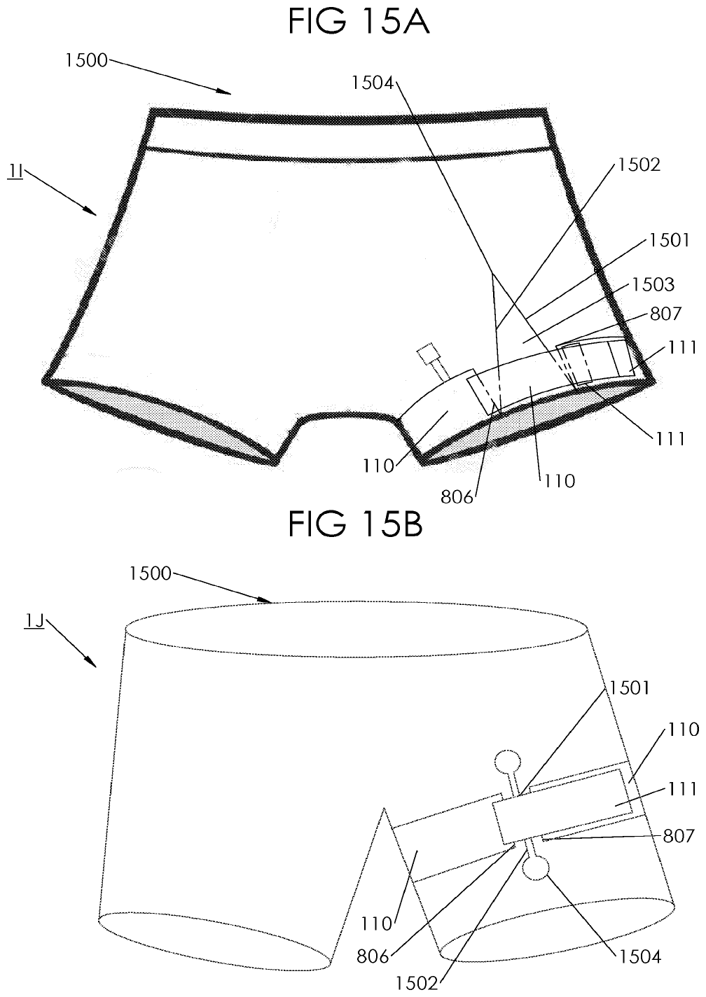

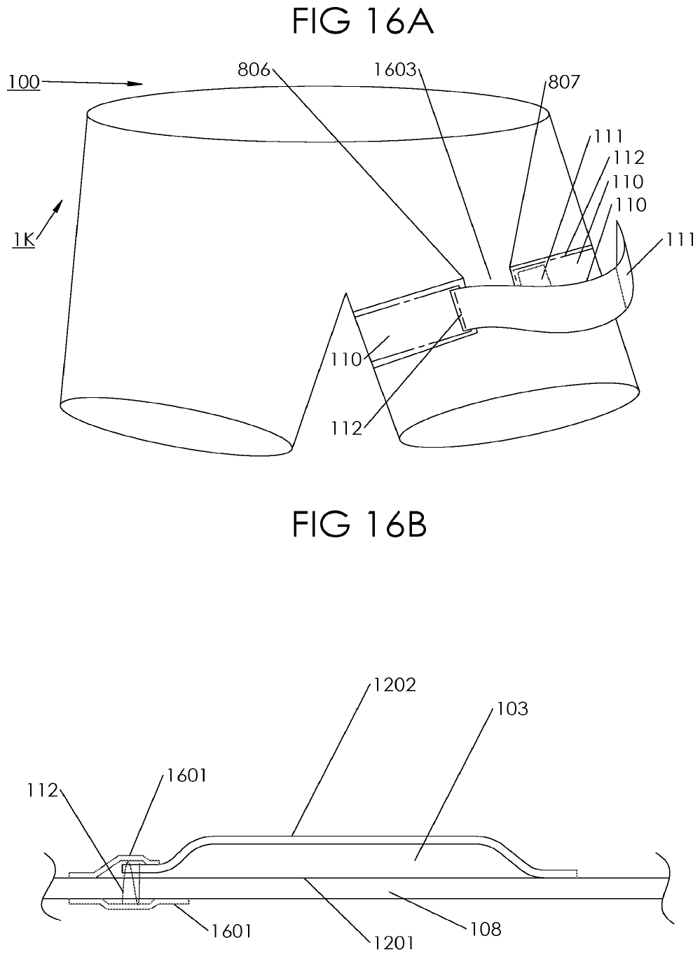

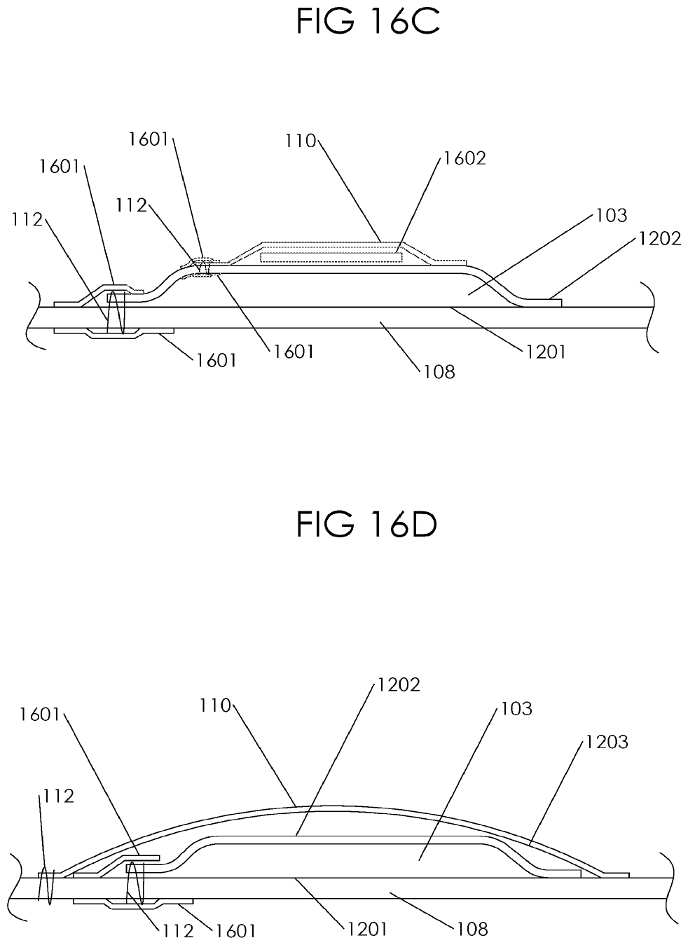

The compression means 105 comprises a first end 806 and a second end 807 as marked in FIG. 16A, FIG. 7E, 8C and FIG. 15A,B, and the compression means 105 may be designed for the first end 806 to overlap with the second end 807, for the first end 806 and second end 807 to displace away from one another, or for the first and second end 807 to both overlap and displace away from one another depending on the limb circumference of the user. Examples of each configuration are described herein. A compression means 105 desirably covers at least 50% of a limb circumference in all positions so the minimum length of the compression means 105 for targeted compression shall be 50% of the maximum designed limb circumference. For example, in the case of a designed limb circumference range of 30 cm-50 cm, a non-stretch compression means 105 that only displaces to larger circumferences has a bladder size would be 25 cm (50% of 50 cm) and so has almost full coverage at the low end of 30 cm, leaving 5 cm open.

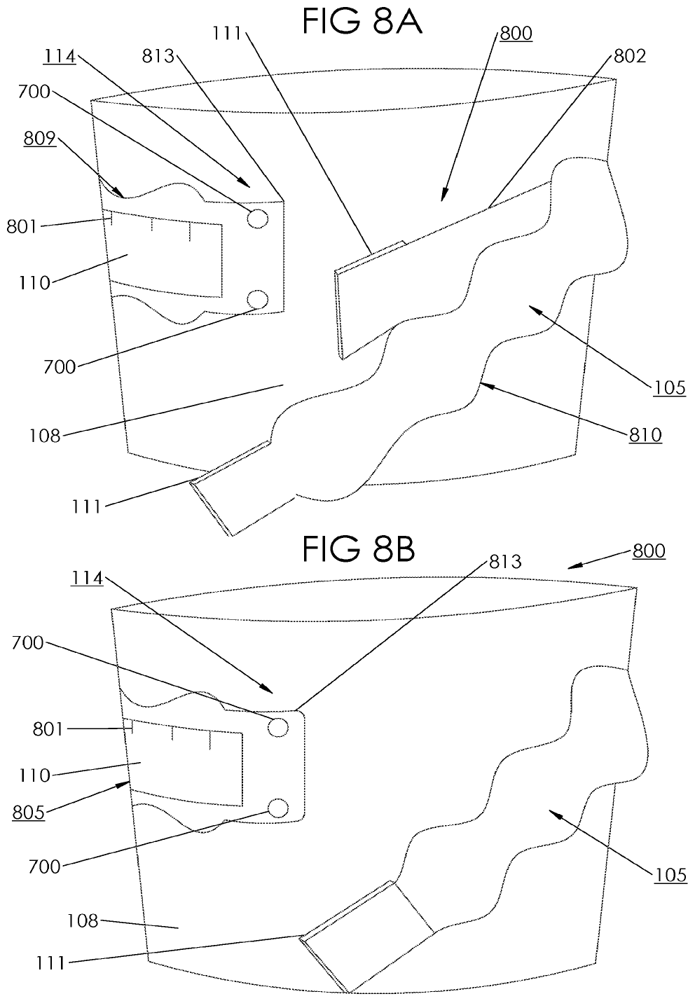

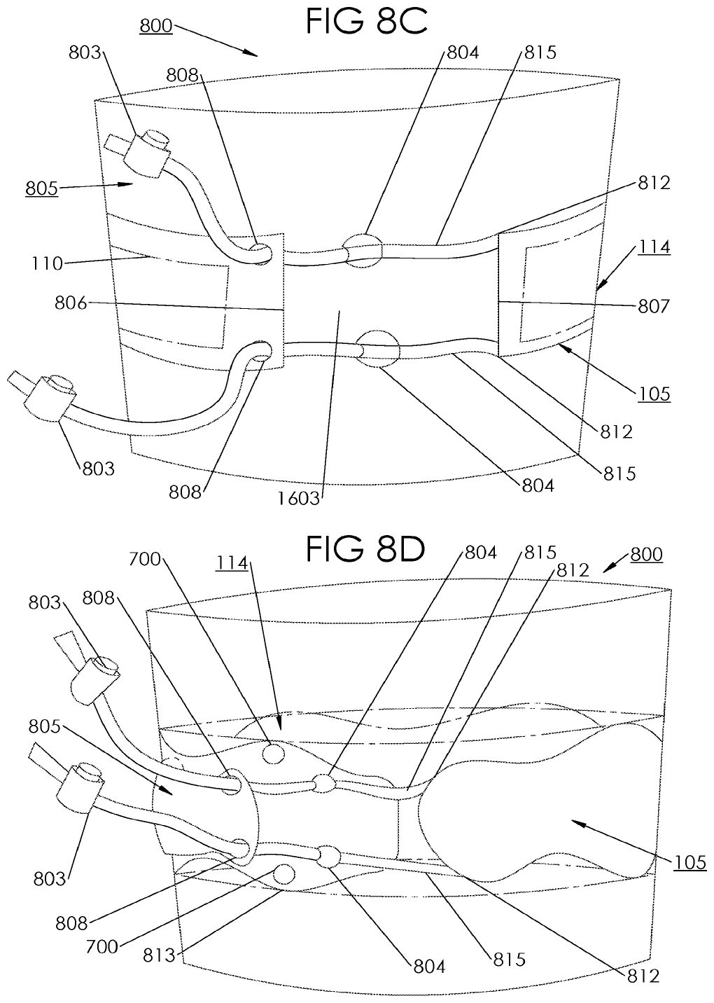

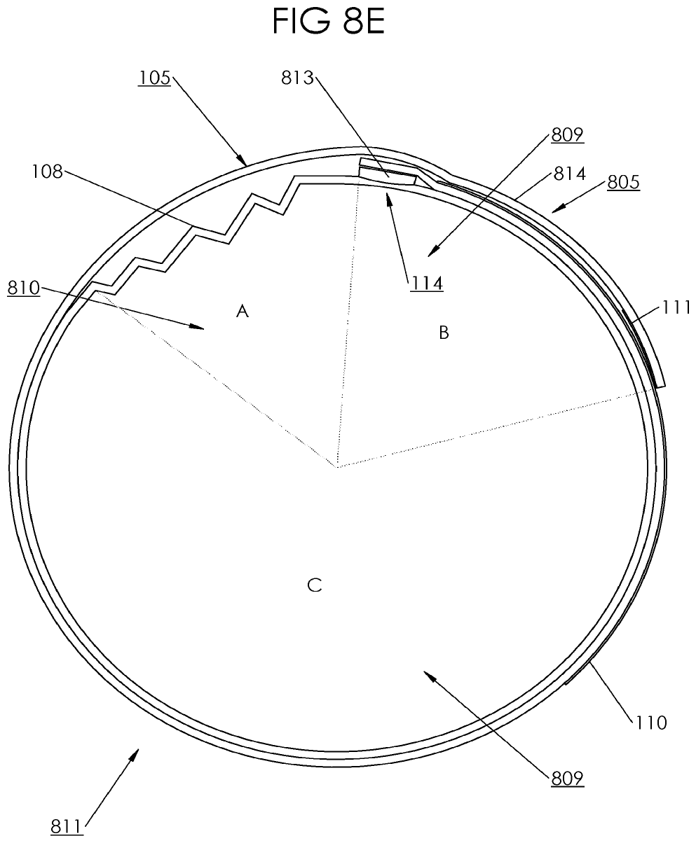

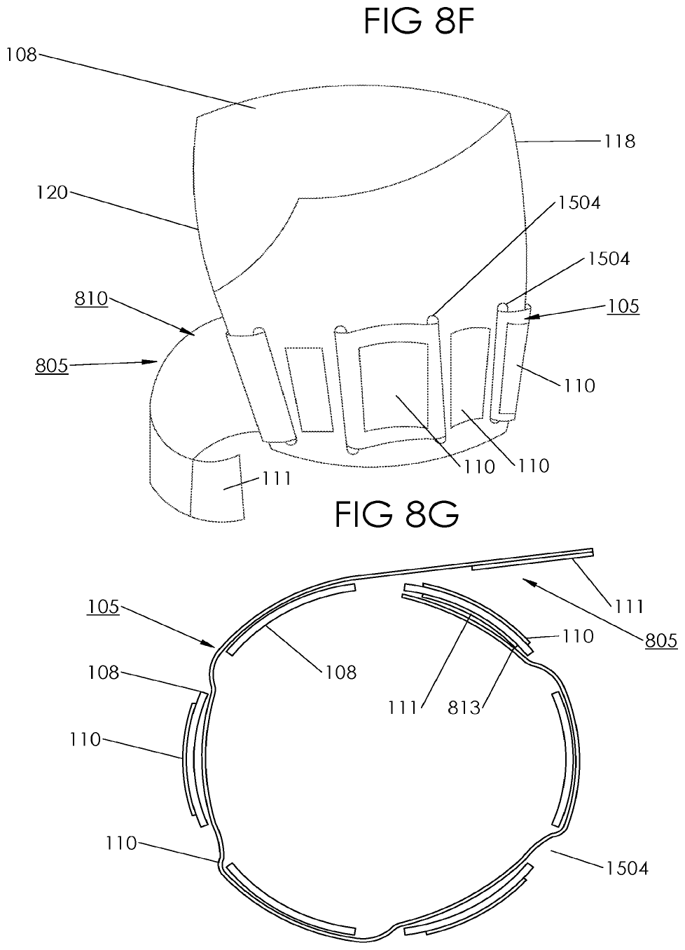

An advantage to overlapping compression means 105 is that a wider range of muscles may be covered for a given compression means 105 length, and in such case the overlapped portion is desirably prevented from displacing laterally relative to the overlapping portion as described herein. It may be advantageous further in this case that a portion of the compression means 105 be non-connected as in FIG. 8E with the conventional clothing element 108 to make the overlapping easier vs having to overlap a portion of the conventional clothing element 108 at the same time if the full length of the compression means 105 is connected as in FIG. 16A. An example of a connected and non-connected portion 810 of the compression means 105 in an overlapping configuration is shown clearly in FIG. 8E in cross section. The reader shall note that even though a compression means 105 may overlap for a certain range of limb sizes, the same compression means 105 may displace apart for larger limb sizes as described further in Alternate Embodiments 7 and 8. The non-connected portion 810 therefore allows independent movement of the conventional clothing element 108 underneath the non-connected portion 810 to increase or decrease in length for extremities of different sizes.

An advantage of a non-overlapping configuration, where an entire portion of the compression means 105 may be a connected portion 809 with a garment 100 as in 16A, is that no further means for preventing lateral displacement of the overlapped portion is generally necessary. Further there is no overlap of the compression means 105 and is therefore less bulky. Further there may be no non-connected portion 810 so there is nothing hanging off the garment 100 that can get caught during washing or just awkward when putting on in general. Further still, in the case of a compression means 105 integrated into the conventional clothing means as described in Alternate Embodiment 2, it may be easier to manufacture a garment 100 where there is no overlapping portion as there is no need to extend an airtight section of the compression means 105 off the surface of the conventional clothing element 108 and patterns and conventional sewing techniques may be maintained. Therefore a non-overlapping configuration of the compression means 105 may lend itself better to cheaper construction and mass production, and overall comfort of the user.

However the reader shall understand that the concepts described in this application for overlapping and non-overlapping configurations, or both, may be adaptable and in combination with any of the other concepts herein such as attachment means 114, conventional clothing garments, adjustable fastening means 805, etc.

The compression means 105 may be comprised of all elastic materials and therefore be elastic itself, or may be non-stretch. In the case the compression means 105 is non-stretch, as described as preferable in prior applications to the applicant, the geometry and attachment means 114 with the conventional clothing element 108 may still allow the garment 100 to accommodate varying limb sizes as described herein. The required change in dimension may come from overlapping of the non-stretch compression means 105, or may come from displacement of a first end 806 and a second end 807 of the compression means 105 away from one another by virtue of a non-connected portion 810 809, or one or more cutout profiles 1504 as described in Alternate Embodiments 7 and 8. In the case of displacement of the first end 806 and second away from one another to increase a bladder end gap 1603, the additional increase in circumference may come from stretch in any underlying conventional clothing element 108 material, being elastic, or may come from one or more cutout profiles 1504 as described later. In the context of this application the reader shall understand the bladder end gap 1603 as illustrated in FIG. 15A,B shall be a portion of the garment 100 that is not connected to the compression means 105 and thus may displace to increase a circumference either by stretching or by opening a cutout profile 1504, or displace to shrink via bunching or overlapping to decrease a circumference around the range of muscles.

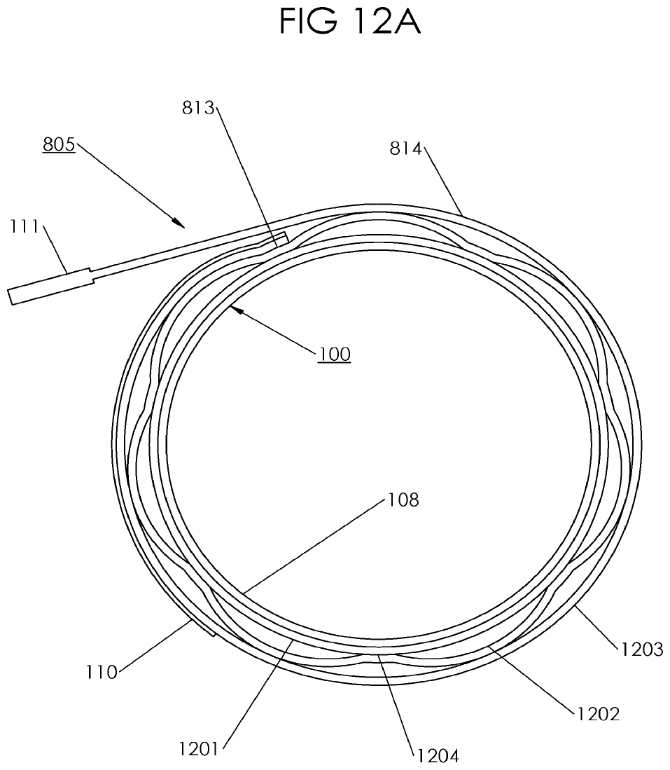

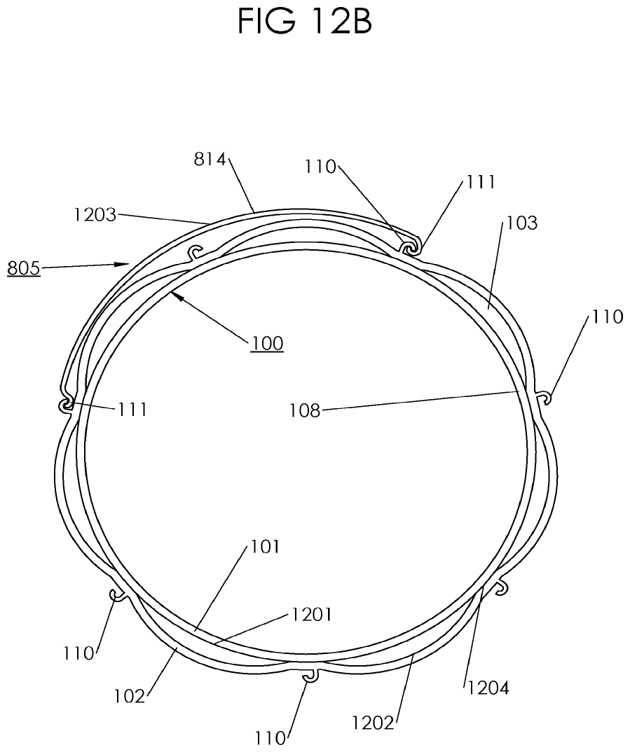

In the case the compression means 105 is itself elastic, it may be attached via suitable elastic means such as bonding or with an elastic stitch, or integrated elastically with the conventional clothing element 108 as described in Alternate Embodiment 2. Elasticity of the compression means 105 has been described in application Ser. No. 15/951,016 regarding an elastic spring element with spring constant between 0.5 and 20 lb/in. In the applicant's invention here, there may be no elastic spring element and the compression means 105 itself being elastic may take on the elastic properties of the spring element. In order to apply sufficient compression in this case several options exist. FIG. 12A and FIG. 16C,D illustrate an outer barrier layer 1203 or stiffener 1602 used to force inflation of the compression means 105 radially inward against the user as in conventional blood pressure cuff designs. Alternatively a barrel inflatable belt may be used, which naturally shrinks in circumference as described in previous applications to the applicant. However, if the barrel inflatable belt is elastic it will still want to expand radially outward to some degree. To combat this, a wider compression means 105, for example up to 15 cm, may be used than would typically be used for blood flow restriction training. A wider compression means 105 has the advantage that less radial pressure is required to restrict a given amount of blood flow, and thus, even though the compression means 105 is not as effective with radial compression because it is elastic, this is ok.

Additionally, by maintaining elasticity, the compression means 105 may be more comfortably worn by the user for long periods of time without having to readjust tension after a session is completed and pressure removed if it elastic on the user's body and applying a lighter compression when not in use.

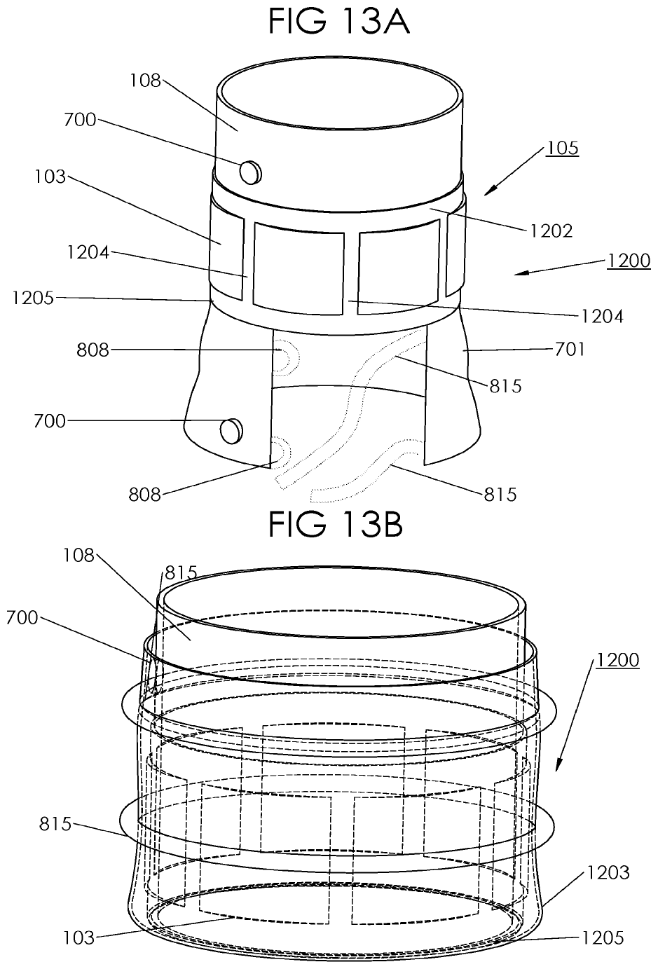

The perimeter of the bladder may be a pure rectangle or may be a series of inflatable chambers 103 as described in prior applications to the applicant and shown in FIG. 13A for reference. While compression means 105 in many of the figures don't show this level of detail the user shall understand that the applicant's invention is not related to a specific construction of compression means 105 and therefore compression means 105 in relation to this specification shall be similarly interpreted broadly. The compression means 105 also comprises an input port 104 as shown in FIG. 2A,B and has been extensively described in prior applications to the applicant. The reader shall note that although not all depictions of the compression means 105 herein show an input port 104, this is for the sake of clarity and all compression means 105 shall be understood to have some form of input port 104 for pressurization.

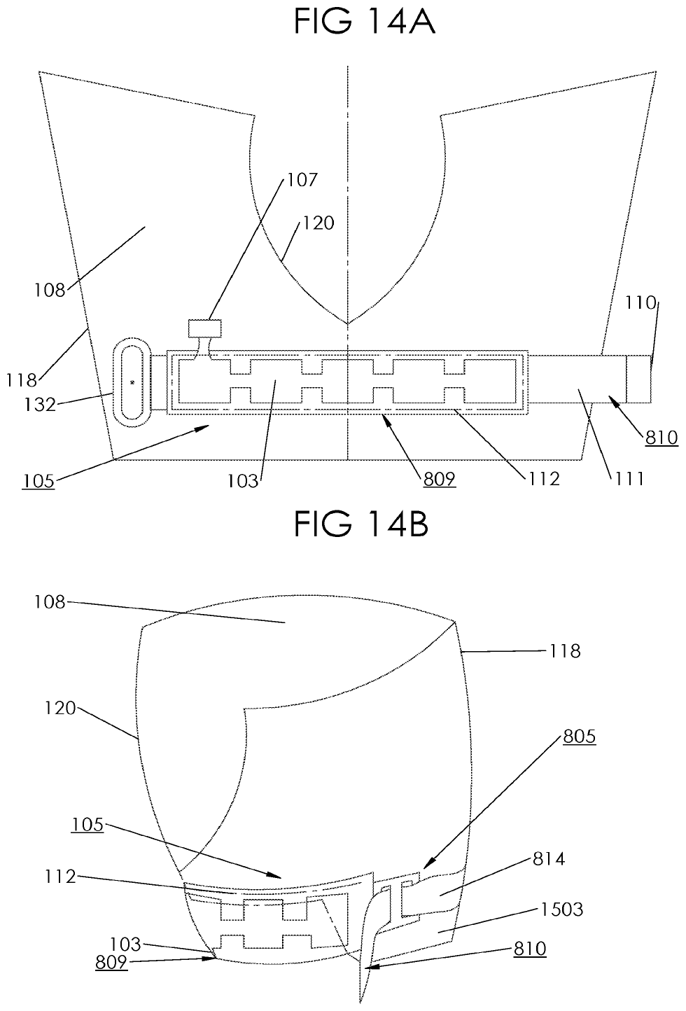

Similarly the compression means 105 may be formed in a fold back style belt as in FIG. 14A, or a straight overlap style belt and both such configurations may be suitably incorporated into the applicant's inventions of this specification.

### Conventional Clothing Element

The foundational element of the BFR garment 100 is a conventional clothing element 108, however the applicant herein provides additional features, such as but limited to: attachment means 114, air pathways. 119, electrical conduits 125, initial tension means 800, and adjustable fastening means 805 that may or may not be added to the conventional clothing element 108, may be removable or permanently attached, and in all cases may also be considered part of the garment 100 for the purposes of this application. The applicant also will describe various features and elements including, but not limited to: control systems 900, inflation means 106, sensing means 906, and external controller 907s that are generally considered part of the device 1 and not considered part of the garment 100, but may also be moved from the device 1 to the garment 100 and integrally connected and considered part of the definition of the garment 100 as a “system” in the spirit of this invention. An alternate embodiment will describe how the operation of the applicant's invention may omit a garment 100 and clothing element as a required component as well.

The conventional clothing element 108 is any type of clothing that may be worn around a user's body and covers or is in proximity to a desired range of muscles to be compressed. The range of muscles to be compressed for the purposes of BFR training are generally the upper thigh and groin region on both legs as well as the upper arm region above the bicep and below the deltoid on both arms. While the applicant recommends these be the muscle regions to compress the reader shall note that the applicant's invention of an integrated BFR garment 100 is not limited to only compressing these areas with compression means 105.

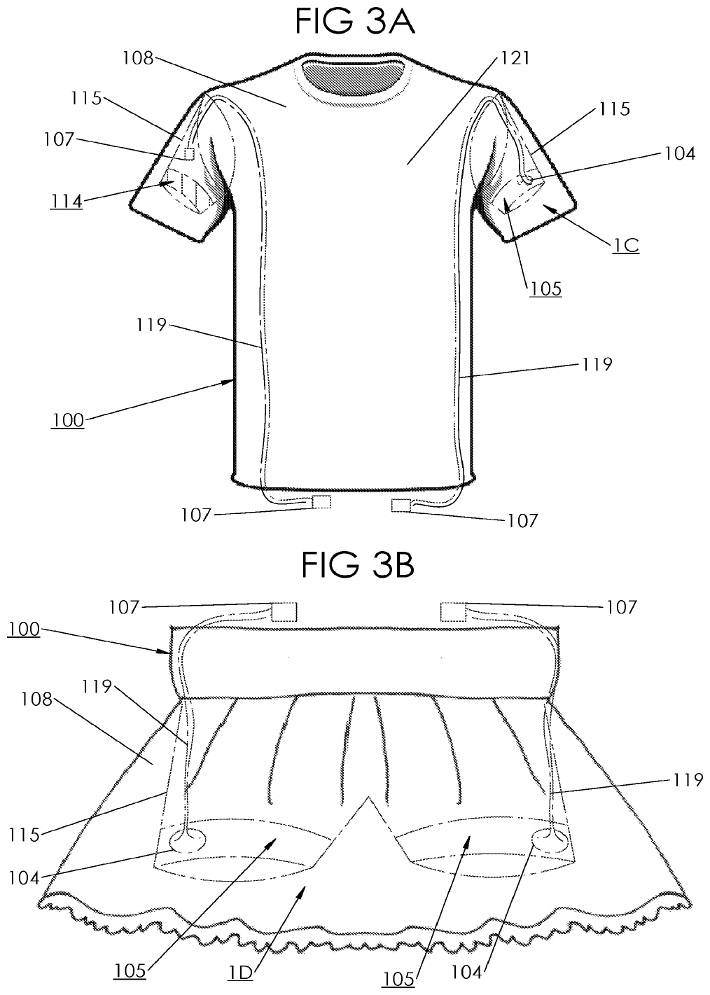

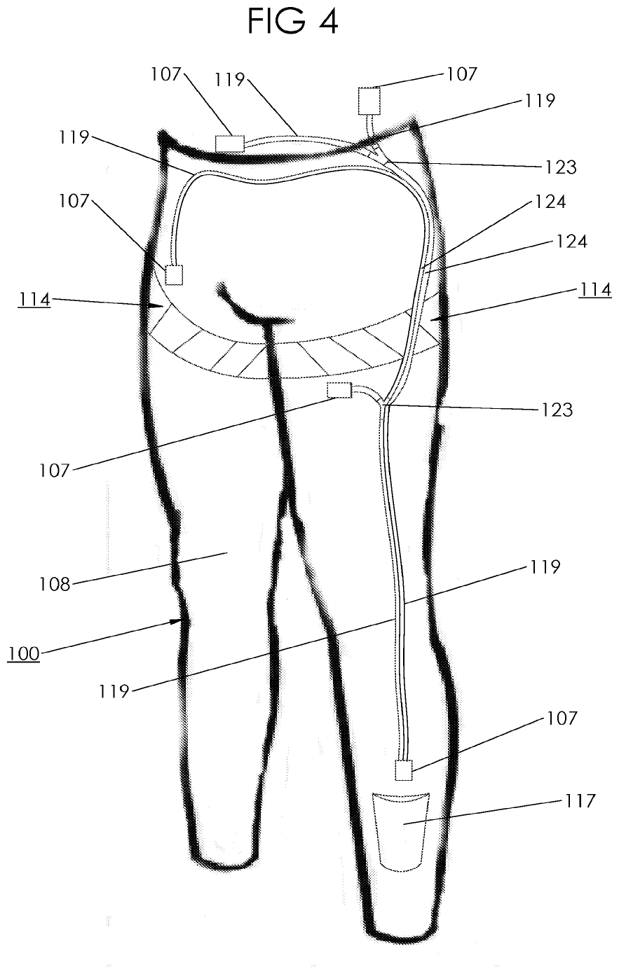

The conventional clothing element 108 therefore may be any of, but not limited to: the short sleeved shirt of FIGS. 1A-C, a long sleeved shirt, the shorts of FIG. 2A-B, the lined shirt of FIG. 3A, the skirt of FIG. 3B, or the long tight fitting pants of FIG. 4. Additionally the conventional clothing element 108 may be a wrap, sleeve, underwear, compression shorts, swimming suits, Speedo's, or any other type of clothing worn by a user during general daily life, or sporting activity. The reader shall understand that the conventional clothing element 108 may be made of any suitable material for creating a garment 100 such as Lycra, cotton, polyester, or any suitable material known in the art of general clothing or athletic wear design. The conventional clothing element 108 may be stetchy or non-stretch, and may further be breathable or may be airtight. The garment 100 may also have portions that are stretchy or non-stretch, breathable or airtight, and need not be comprised entirely of one material or material property. The reader shall understand the conventional clothing element 108 to be configured or fabricated or modified according to the needs of this preferred embodiment and the other alternate embodiments where appropriate.

The conventional clothing element 108 is preferably tight fitting and stretches to fit over the user's body, but may also be loose fitting as in an undershirt and may be minimally elastic. A tight fitting undershirt for example is preferable to a loose fitting T-shirt, but the fit on the user is not a critical element to the invention and application will describe out the invention herein accommodates both options. Tight fitting heretofore is used to describe a garment 100 that expands in circumference to accommodate a user's body and loose fitting is used to describe a garment 100 that is larger than the user's body such that an air gap exists between the user's skin and the inside surface of the garment. The reader shall note that a garment 100 may be both a loose fitting and tight fitting garment 100 as described in Alternate Embodiment 8, whereby the garment 100 is designed for median size range such that it is loose fitting for a certain range of smaller users and tight fitting for a second set of larger users.

The conventional clothing element 108 may be designed in different sizes to accommodate different types of users. For example a S, M, L, XL etc. size range may be used, or a limb circumference may be used such as that suggested in the applicant's prior invention on inflatable belts. The reader shall understand that sizing is well understood in the art, and adaptations specific to BFR training may be made, and may also be altered based on the characteristics of the herein described invention. The conventional clothing element 108 may further accommodate a wider range of sizes by comprising an optional cutout profile 1504 as described further in Alternate Embodiments 7&8.

The conventional clothing element 108 may be designed for use without an additional layer covering the conventional clothing element 108 as in Alternate Embodiment 1, or may be designed and intended to be worn underneath another piece of clothing, or even on top of another piece of clothing. Where the conventional clothing element 108 serves as an undergarment, or the garment 100 incorporates a liner 115 as in Alternate Embodiment 1, this may be advantageous in terms of hiding the BFR garment 100 from public view so that there is not an element of distraction or attention drawn to the user for those who are shy and don't want to be seen walking around while doing BFR training.

### Air Pathways

The device 1 preferably comprises one or more air pathways. 119, and the conventional clothing element 108 is preferably in communication with one or more air pathways 119 as shown in FIG. 1A, which transport air from a first point 128 on the conventional clothing element 108 to a second point 129 on the conventional clothing element 108.

The air pathways 119 are composed of substantially airtight material such as a section of polyurethane, latex or PVC tubing, or alternatively formed by creating a channel along the garment 100 by, for example bonding a first layer of polyurethane, or suitable thermoplastic material to the conventional clothing element 108, and then bonding a second layer along the edges only such that the middle section between the two layers is un-bonded, and may expand to form a channel to allow air to pass through. Partially bonding layers of flat thermoplastic sheet to the conventional clothing element 108 may have the advantage that when the air pathway 119 is not inflated it lies flat and flexible against the skin, vs a tube which may protrude farther off the surface of the conventional clothing element 108. In the case air pathway 119 are integrally formed with the conventional clothing element 108 and garment 100, the air pathways 119 may be considered part of the garment 100 and the device 1 then attachable to the air pathways. Therein lies an illustration of how one element, an air pathway 119 in this case, can be moved between the device 1 and the garment 100 for the purpose of this specification. The reader shall note that several examples of how to create an air pathway 119 have been given, but the invention shall not be limited to these and any suitable means for allowing air to move between points on the BFR garment 100 shall be considered within the scope of this invention.

The air pathways 119 may start on any point on the BFR garment 100 and may stop on any point on the BFR garment. The important aspect is that they are constructed and placed suitably to connect a compression means 105 with an inflation means 106 in such a way that the compression means 105 may be activated and controlled to apply appropriate compression, otherwise termed the working compression level, to the range of muscles at the appropriate point in time for effective BFR training by the user. For example the air pathways 119 may start on the regions of the upper thigh, hip, buttocks, groin, or waist and in proximity of the compression means 105 for a lower body BFR garment. These same air pathways 119 may terminate near the ankles, waist, hip, stomach, neck, lower back, or other region where a control system 900 preferably containing the inflation means 106 may be located.

For an upper body BFR garment 100, the air pathways 119 may originate near the region of the upper arm, shoulder, scapula, pectoral, back, or side region of the BFR garment 100 and may terminate near the ankles, waist, hip, stomach, neck, lower back, or other region where the control system 900 preferably containing the inflation means 106 may be located. The reader shall understand the concept and purpose of the air pathway 119 and not limit the invention only to the regions discussed above.

The routing of the air pathways 119 may similarly be done in a multitude of ways. It is desirable that the air pathways 119 generally follow the seam lines in the conventional clothing element 108 as much as possible for maximum comfort of the user. These may be an out-seam 118 of a shirt as in FIG. 1B, or an in-seam of pants as in FIG. 2A (note the air pathway 119 is not depicted as running along the inseam 120 in FIG. 2A, but the inseam 120 is illustrated) as but two examples. The air pathway 119 may be routed around different faces and surfaces of the garment 100, for example from a front face 121 of a sleeve 116 as shown in FIG. 1C over the shoulder and down a back face 122 of the conventional clothing element 108.

The air pathway 119 may also route from an internal surface (being the inside of the garment) of the garment 100 to an external surface (being the outside of the garment), but may also route exclusively on the internal surface or external surface. Routing along an internal surface may be advantageous in that the air pathway 119 is hidden from view and routing along an external surface may be advantageous in that the air pathway 119 is more easily accessed and may be less abrasive against the user's body. Other suitable areas for routing the air pathways 119 may be, but are not limited to a front face 121, back face 122, inseam 120, out-seam 118, side-seam, or other suitable area of the BFR garment. The reader shall understand the terms front face 121 and back face 122 equate to the front and back parts of piece of clothing and inseam 120 and out-seam 118 are generally understood in the art of sewing to be like the inseam 120 of pants or an outer facing seam of pants or shirt for example. The reader shall understand it is desirable that the routing and location of the air pathways 119 minimally restrict movement or disturb the user during normal daily activity and also avoid pinching and closing off of the air pathway 119 during movement and use.

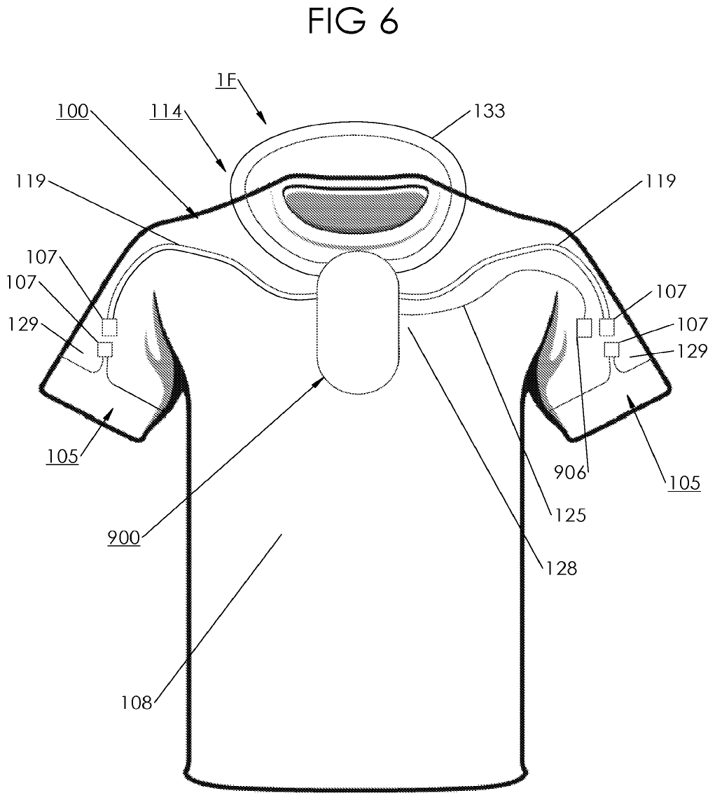

The air pathway 119 may be integrally formed, as in FIG. 1A-B, with the BFR garment 100 during the manufacturing process such that it may not be removed, or it may be removable and attached via attachment means 114 shown as loop 127 and sleeve 116 in FIG. 1C. Similar to the layered air pathway 119 concept above, tubing or other conventional air pathway 119 may be bonded, glued, stitched in, or otherwise permanently connected to the conventional clothing element 108 in order to minimize hassle for the user in taking components in and out for washing or cleaning for example. Alternatively the air pathway 119 may be removable as shown in FIG. 1C (or integrated with the device 1 as in FIG. 6), wherein the air pathway 119, shown as a tube, is passed through a loop 127 and a sleeve 116 in order to secure and route the air pathway 119 along the BFR garment 100 between the first point 128 and second point 129. An air pathway 119 that is removable may have the advantage that it can be easily replaced if a hole is punctured for example, may be more easily cleaned, and may further simplify the manufacturing process and open up a broader range of manufacturers who may not have the capability to bonding air pathways 119 together with conventional clothing element 108s.

Multiple air pathways 119 may be joined with an air pathway junction 123, an example of which is shown in FIG. 2B. An advantage of the air pathway junction 123 is reduction in connectors, improved reliability, even application of pressures, and a larger reservoir 124 to buffer pressure spikes from muscle contractions. The air pathway junction 123 is described further in Alternate Embodiment 6, and as with the air pathway 119 may comprise part of the device 1, garment 100, or be an independent element.

The reader shall understand that all the figures shown may be configured to work with either an integrally formed air pathway 119 or a separable air pathway 119 and the specific implementations in the figures are illustrative only and not meant to limit to scope of the invention. Similar, where multiple means of securing an air pathway 119 to the BFR garment 100 are shown, for example by loop 127 and sleeve 116 in FIG. 1C, the reader shall understand that only one such means may be used without departing from the concept of providing enough support and routing guidance to get the air between two points in a comfortable, effective, and reliable manner. Therefore the quantity and length of loops 127 and sleeves 116, or comparable means, shall be whatever is necessary to guide and support the air pathway 119 in the desired manner.

The reader shall also note that not all figures depict an air pathway 119 but that this does not mean an air pathway 119 cannot, or is not used with that particular figure or embodiment or as part of the device 1 or the garment. Therefore omissions of air pathways 119 for the sake of clarity do not prevent air pathways 119 from being used with such embodiments or figures in this application.

Finally the reader shall note that the air pathway 119 is not a requirement of the applicant's invention for a device 1 and garment 100, and each compression means 105 may be individually inflated by an inflation means 106, however the air pathways 119 offer a convenient method for efficiently, and potentially automatically, inflating multiple compression means 105 of the garment.

### Connection Means

At the ends of each air pathway 119 are preferably disposed one or more connection means 107. While the air pathway 119 may be integrally formed with the device 1A and compression means 105 as shown in FIG. 2A for example, thereby forgoing the need for a connection means 107 between the compression means 105 and the air pathway 119, this may not be desirable. In particular, for compression means 105 that are separable from the BFR garment 100, a connection means 107 allows the air pathway 119 to connect to the compression means 105 after the compression means 105 is installed. This means the air pathway 119 has the flexibility of being attachable (permanently or removably) to the conventional clothing element 108 independently of the compression means 105. The connection means 107 may be any suitable means that connects two substantially airtight areas together such as a quick disconnect valve, a bayonet coupling, a hose barb, or any other means known in the art. The connection means 107 may comprise a one-way valve to maintain pressure on one side of the connection means 107, or otherwise be through-flow. Connection means 107 may also be disposed on the end meant for attaching to an inflation means 106 or control system 900. Alternatively the control system 900 and/or inflation means 106 may be permanently connected to the air pathway 119 as in FIG. 6, but it may be desirable to keep the two elements separate for ease of installment, cleaning, or operation without entanglement of the air pathway.

### Electrical Conduit

Similar to the air pathway 119 as shown in FIG. 2A, the device 1A or garment 100 may comprise one or more electrical conduits 125 and the electrical conduits 125 may be provided between two points on the device 1A, or if a garment 100 is provided, between two points on the garment 100 as shown in FIG. 1A, 2A, 6. The electrical conduit 125 may have similar properties and limitations as referenced in the air pathway 119 description. Rather than transporting air however, the electrical conduit 125 transports electrical signals which may be data 908 signals or supply power among other options. The electrical conduit 125 may take the form of a wire, cable, strand of fabric in the case of conductive clothing, or other suitable conduction mediums for getting signal and/or power from one position on the body to another. As with the air pathway 119, the electrical conduit 125 may be integrally formed and bonded, sewing, woven, or otherwise permanently connected to the garment 100, or may be separate from the garment 100 and housed and routed through one or more sleeves 116, loops 127, or equivalent securing and routing members.

Additionally, and also similarly to the air pathway 119 the electrical conduit 125 may be terminated with one or more connection means 107 for electrical connection to another member such as a sensing means 906 or the control system 900. The reader shall note there are many suitable types of connection means 107 known in the art of electronics design, and that in the purposes of this application, the connection means 107 shall extend to both a connector for pneumatic connections as well as electrical connections. Alternatively to connection means 107, the electrical conduit 125 may be integrally formed with either or both of a sensing means 906 or control system 900, for example via direct soldering.

Additionally, the electrical conduit 125 may run alongside the air pathway 119 or may run to a different location on the BFR garment. For example if an EKG signal is desired for heart rate variability data 908 collection, which is useful as described in a previous application to the applicant, a sensing means 906 may be placed near the heart and down by the side of the stomach and these locations may be different than the routing of the air pathway. The reader shall therefore understand that while much of the description of the air pathway 119 in terms of fixation, location, presence, connection means 107, etc. applies to the electrical conduit 125, the electrical conduit 125 is its own element and shall be treated independently in the context of this invention and designs disclosed herein. The reader shall also understand that RF may be used to route data 908 in place of an electrical conduit 125 and RF shall also therefore be considered an electrical conduit 125 for the purposes of this application.

The reader shall also note that not all figures depict an electrical conduit 125, but that this does not mean an electrical conduit 125 cannot, or is not used with that particular figure or embodiment. Therefore omissions of electrical conduit 125 for the sake of clarity do not prevent electrical conduit 125 from being used with such embodiments in this application.

### Attachment Means—for Compression Means 105

An attachment means 114 may be in communication with either or both of the device 1 and the garment 100 to allow removal of the device 1 and compression means 105 as in FIG. 8A,B from a garment 100 if provided, or to permanently connect and compression means 105 to the garment 100 as in the preferred embodiment of FIG. 16A. FIGS. 7A-E, 8A-G depict a variety of attachment means 114 for securing a separable compression means 105 to the conventional clothing element 108 as part of the garment. FIGS. 7A-E show only a section of the conventional clothing element 108, illustrated as a cylindrical shape, and the reader shall understand the cylindrical shape meant to be placed over a range of muscles as previously described. The attachment means 114 may be considered to be any method, components, or combination thereof that accomplishes the goal of securing the compression means 105 to the conventional clothing element 108, and the securing may be permanent or may be temporary depending on the attachment means 114. Various embodiments will be described below, and the reader shall recognize that all such variations may be considered suitable and adaptable in part, or in combination, to the various forms of BFR garments 100 disclosed in this application. The reader shall also understand that the attachment means 114 may further direct and define the orientation and location of the compression means 105 on the user. For example, where button snaps 700 are used, the location of the button snaps may define a starting location for one end of the compression means 105, which positions the compression means 105 in a fixed spot and a fixed orientation on the garment.

Therefore, the term attachment means 114 shall be interpreted broadly and extended beyond the illustrations in the figures discussed and apply to any other types of attachment known to those skilled in the art.

If provided in communication with the garment 100, the attachment means 114 is located on the conventional clothing means in the vicinity of the range of muscles to be compressed and preferably over the range of muscles to be compressed. Where the user size may differ, the conventional clothing element 108 may also be configured to be in different sizes and therefore the scale and location of the attachment means 114 similarly altered. Alternatively, as in FIG. 8D, the attachment means 114 itself may be adjustably connected with the conventional clothing means via suitable methods discussed herein such as, but not limited to, hook and loop fastener, loops 127, sleeves 116, glue, tape, adhesive, sewing, buckles and straps, etc.

Where the conventional clothing element 108 is preferably stretchable, as in a tight fitting garment 100, the location of the attachment means 114 on the user's extremity may also adjust due to the stretching the conventional clothing element 108 as in the button snaps 700 of FIG. 7A. This stretching action preferably happens automatically as the conventional clothing element 108 naturally expands in order to be tight fitting to the body, like a compression garment. For example in 7A, a larger limb will stretch the portion of the conventional clothing element 108 in between the button snaps 700 and displace each button snap further away from its neighbor the larger the limb size. In this case, there may be more button snaps 700 disposed on the compression means 105 versus the conventional clothing element 108 for better alignment across multiple user sizes.

Where the compression means 105 is non-stretch, as in the preferred embodiment of FIG. 16A, a conventional straight stitch may be used. In FIG. 16A the compression means 105 is disposed around a portion of the range of muscles a portion of the conventional clothing element 108, which is elastic, is left uncovered by the attachment means 114 and compression means 105. The non-stretch nature of the attachment means 114 is appropriate because the compression means 105 itself is non-stretch, however if the compression means 105 were elastic, the straight stitch could be an elastic stitch. In FIG. 16A, because the attachment means 114 is not disposed around the entire circumference, a section of the conventional clothing element 108 can stretch and adjust in length. The attachment means 114 in FIG. 16A is permanent.

On the other hand, where sewing is used as the attachment means 114 for permanently connecting a compression means 105 to the conventional clothing element 108, the type of stitch may be used to affect the elastic behavior of the garment. For example where the compression means 105 is itself elastic, and circumferential expansion is desired, a zig-zag, overlock, or other expandable stitch may attach the compression means 105. The attachment means 114 in the context of allowing a garment 100 to expand or contract in dimension is described further in this application.

Optional location means (not shown) or measurement means may be provided in order to locate and space or place the attachment means 114 in the correct location in relation to an anatomical landmark (not shown) such as a hip bone or shoulder bone. However, in the applicant's recommendation for placement of the compression means 105, which is as high upon the arm as possible and as high up on the legs as possible, the location is naturally driven by the user simply by donning the BFR garment 100 and letting the conventional clothing element 108 stretch as it is independently designed to do. The attachment means 114 therefore, in additional to locating the compression means 105 on the user's extremity, may allow for sufficient expansion of the conventional clothing element 108 when being worn by a user. Additionally, while the attachment means 114 may provide additional construction or behavior benefits to the BFR garment 100, the basic purpose is to connect a compression means 105, air pathway 119, or other element with the conventional clothing element 108 without interfering with the ability to apply initial tension or working compressive level to the range of muscles, and the variations disclosed below shall be understood to accomplish this basic design goal.

### Attachment Means—For Inflation Means 106/Control System 900

Attachment means 114 in the context of this specification not only relates to a means of attaching the compression means 105 to the garment 100 or user's body, but also a means of attaching other elements of the device 1 such as, but not limited to: a control system 900, inflation means 106, air pathway 119, electrical conduit 125 to the user's body. Attachment means 114 as it relates to non-compression means 105 items shall therefore also benefit from the same broad interpretation as was described above related to attaching the compression means 105 to the conventional clothing element 108 and user's body.

FIGS. 4, 5, 6 depict several additional variations of attachment means 114 as they relate to attaching the device 1, comprising control system 900 and/or inflation means 106 for example, to the conventional clothing element 108 or user's body. FIG. 4 depicts a pocket 117 into which the control system 900 or inflation means 106 may be slipped. The pocket 117 may have an optional locking means (not shown) like a button snap 700 to close the opening or simply rely on tension and friction and/or gravity to prevent the contents from falling out. The pocket 117 may be placed in any convenient location on the conventional clothing element 108, for example the ankle area of a pair of tight fitting pants as shown in FIG. 4. The ankle area may be particularly suitable as this may be covered easily with working slacks or pants and be imperceptible to outside observers, not unlike an ankle bracelet for those on house arrest. Additionally, when combined with other embodiments for the inflation mechanism the inflation may be actuated by a foot step of a user to pump air into the compression means 105 as in Alternate Embodiment 2, or external reservoir 909 and therefore the controller may be suitably located near the pump source as shown in FIG. 4.

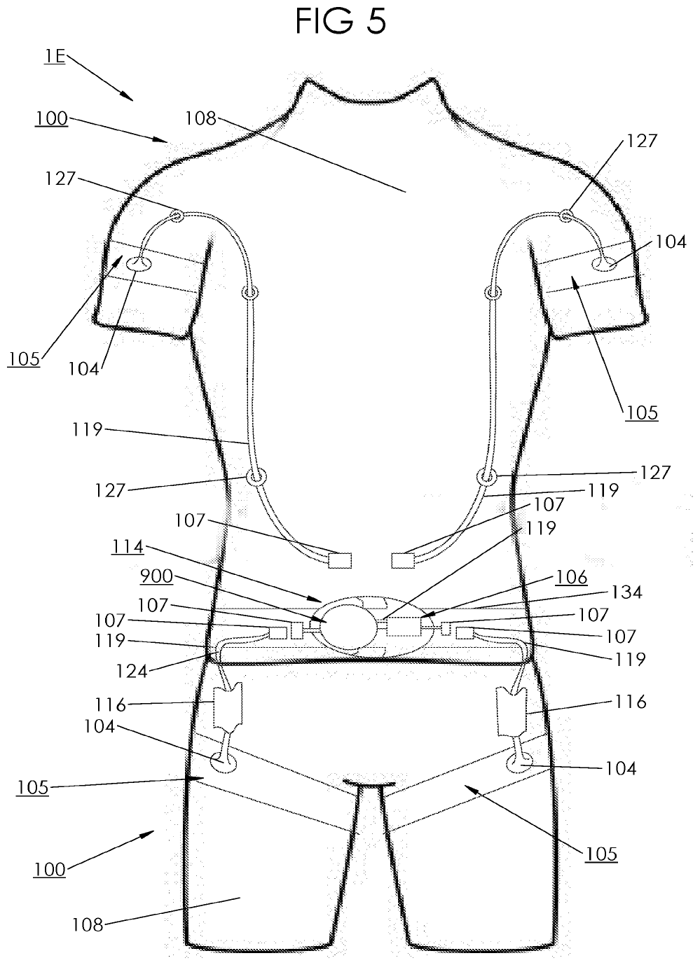

FIG. 5 depicts device 1E comprising multiple compression means 105 and air pathways 119 in communication with an upper and lower body garment 100, and further comprising a control system 900 attachable to a belt 134 worn by a user that may not be connected to the conventional clothing element 108 and simply worn as a normal belt, or otherwise hidden under clothing. The belt 134 may be integrated with the control system 900 and/or inflation means 106 similar to how a heart rate monitor chest strap is created, or the control system and/or inflation means may be attachable to the belt. In the case of integration, the control system 900 or inflation means 106 may have an elastic, or otherwise adjustable, band connected to each side of it such that the band suspends and secures the inflation means 106 or control system 900 at an appropriate location near the user's 90 waist for appropriate connection of the optional air pathways 119 and electrical conduits 125. In the case the inflation means 106 and/or control system 900 is NOT integrated, the attachment means 114 may constitute a harness in the form of a clip in brace as shown, or other mechanism for mechanically fastening the control system 900 and/or inflation means 106 to the attachment means 114, which in turn is secured to the user in an appropriate location. The belt 134 of FIG. 5 may be integrated with one of the conventional clothing element 108s or may remain a separate item that may or may not be secured with an additional attachment means 114 such as the loop 127 (acting as belt loops) described earlier. FIG. 5 shows one variation of the control system 900 where the inflation means 106 is outside an enclosure of the other components in the control system and is in communication with the control system via an electrical conduit 125 so that the control system may control the compression means. FIG. 5 also illustrates how the control system 900 may have connection means 107 (only one is shown for clarify, but multiple connection means may be provided) for connecting an air pathway 119 to the control system, and as shown in FIG. 5, the control system may also be in communication with the inflation means via an air pathway, separable, or integrally formed as shown. The air pathway 119 between the inflation means and the control system 900 may transmit air to additional compression means through the control system as shown, the control system therein serving the function of an air pathway junction 123, and the control system may also sense the pressure in the reservoir 124 via a sensing means 906 (not shown) reading the pressure in the reservoir. FIG. 5 also illustrates how the compression means 105 may be directly in communication and even integrally connected with the inflation means 106.

FIG. 6 shows a device 1F comprising an attachment means 114 in the form of a neck strap 133 for wearing around a user's neck and for holding a control system 900 and/or inflation means 106 for subsequent connection to either optional air pathways 119 and/or electrical conduits 125 via connection means 107. The neck strap 133 may standalone that, like the belt 134, may be a control system 900 and/or inflation means 106 integrated with a strap, or may simply be an attachment means 114 in the form of a harness into which a control system 900 and/or inflation means 106 is clipped or otherwise attached. The neck strap 133 is preferably free floating such that it may be worn on the inside or outside of the user's clothing. The air pathway 119 shown may be integrated into the BFR garment 100 or simply be integrated into the neck strap 133 as shown in FIG. 6. The neck strap 133 therefore preferably serves as a separate wearable item that the user can attach to their BFR garment 100 for supplying air and/or electrical signals and power to the compression means 105 which is in communication with the conventional clothing element 108.

Connectors attached to air pathways 119 and or electrical conduits 125, may be terminated in the vicinity of the attachment means 114 for the inflation means 106 or control system 900 as previously described so that they may be easily connected and air and/or electrical signals passed to the control system 900 in order to apply appropriate compressive forces to the range of muscles. The neck strap 133 for FIG. 6 is shown in communication with two compression means, but the user shall understand additional air pathways 119 may be added to the device 1E for the legs.

### Initial Tension Means—

The applicant has filed prior applications discussing the reason and importance of initially tensioning the compression means 105 to a pre-determined and consistent level. In summary, the pre-tension applied by an initial tension means 800 should be consistent from one session to the next because it sets the baseline off of which the working compression level is generated. If the initial tension is too loose, or inconsistent, then the pressure settings used by the user or control system 900 will similarly be inconsistent and may be too tight so as to be dangerous or too loose so as to be ineffective. The initial tension means 800 is ideally prescriptive and repeatable in its own right and the applicant's prior inventions aim to solve this problem with other prior art. The initial tension means 800 may be part of the device 1 if integrated into the compression means 105, part of the garment 100 if integrated with the conventional clothing element 108, or with both if the garment 100 is integrated with the device 1.

The reader shall note that it may be desirable that the initial tension applied be such that a minimal amount of blood flow is restricted and the user is not put into an uncomfortable position. The degree to which the compression means 105 may further be inflated to apply a compressive force will dictate how loose this initial compression level can be. For example, if a standard rectangular inflatable bladder is used and the bladder construction allows for significant expansion due excess material, or an elastic inner bladder layer 1201 for example, then the initial tension setting can be a more loose fit setting. Conversely, if the rectangular bladder does not accommodate significantly expansion potential because it is narrow and/or elastic, then the initial tension setting must be tighter fit in order to provide enough further compressive capability to reach the required working compressive force on the range of muscles. In the case of a barrel inflatable design such as that invented previously by the applicant, the bladder itself will shrink in size so the initial tension setting need only be sufficiently tight that the shrinkage of the bladder in circumference coupled with the inward pressure from expanded inflatable chambers 103 may achieve the required compressive force on the range of muscles. The barrel inflatable belt design may be desirable in this respect because it can be more “loose fit” generally as the initial tension, and therefore provide a high level of comfort or “normal” feeling to the user when not in use throughout the day, and simply inflating it to the closed position to apply the working compressive force is achievable without further adjustment of the initial tension to start the belt “tighter” on the limb.

In the preferred embodiment of FIG. 16A, the initial tension is applied by the stretch of the material of the conventional clothing element 108 itself via expansion of the bladder end gap 1603, and no additional components are necessary. This adds simplicity in construction and still may provide enough adjustment for an adequate size range. As the compression means 105 in 16A is non-stretch, all of the adjustment must come from the bladder end gap 1603, by either stretching to increase circumference or overlapping/bunching the fabric to reduce the circumference or bunch up. In FIG. 16A a piece of first fastening means 110 shown as loop fastener is disposed on an outer surface of the compression means 105 starting at a second end 807, and a piece of second fastening means 111 shown as loop fastener is disposed around a first end 806 of the compression means 105, overlaps the bladder end gap 1603, and is securable to first fastening mean to fix the open position 811. The initial tension as stated was supplied by the stretch in the fabric of the bladder end gap 1603, and the closure of the adjustable fastening means 805 (first fastening means 110 to second fastening means 111) maintains the initial compression level and prepares the belt for inflation.

To augment the available size range, a portion of the compression means 105 may further be configured to be a non-connected portion 810 similar to described in relation to FIG. 8E, which is essentially expanding the length of the bladder end gap 1603. If a bladder end gap 1603 can stretch 100% for example, a bladder end gap 1603 of 5 cm has a 10 cm limb adjustment range if it is tight fitting. If another 5 cm is non-connected, the bladder end gap 1603 length effectively increases to 10 cm and the available size range is doubled. FIG. 8E illustrates what happens in the case where a compression means 105 is attached to the conventional clothing means around a portion of the circumference and this attachment may be as an integrally formed junction or using attachment means 114 described previously and shown as anchor point 813 in FIG. 8E. The more of the compression means 105 that can be attached to, or integrated with the conventional clothing element 108, the more usable the BFR garment 100 is because there are less loose items or parts of items that are dangling off and can get caught on other garments 100 or in the washing machine etc.

However there are competing aspects of the design as mentioned previously of the compression means 105 and the ability of the BFR garment 100 to accommodate individuals of different limb girths. Namely, the conventional clothing element 108 must stretch in the case of a tight fitting style, or must bunch or overlap, in the case of a loose fitting style in order to allow the compression means 105 to achieve a proper initial tension around the user's limb. The compression means 105 by contrast is preferably non-stretch (or at least an outer most layer is non-stretch) because free expansion of the compression means 105 under inflation detracts from the ability of the compression means 105 to apply sufficient compressive force to the range of muscles. Similarly the compression means 105 cannot bunch because then it cannot carry tension required to apply any compressive force at all. Therefore, a portion of the compression means 105 and the conventional clothing means may be a non-connected portion 810 shown in FIG. 8E such that the compression means 105 can be set to an appropriate initial tension after the BFR garment 100 is donned by the user. The reader shall note the applicant has disclosed designs for an expanding compression means 105 in the form of an expandable bladder, but even in this case an outer barrier layer 1203 is provided, which is also considered part of the compression means 105. The outer barrier layer 1203 is disconnected around at least a portion of the conventional clothing element 108 therefore as shown FIGS. 12A,B, 13A,B, 16D.

FIG. 8E shows a section view of a loose fitting conventional clothing means, tensioned to be smaller than its nominal circumference thereby causing bunching of the conventional clothing element 108. The compression means 105 is shown as an inflatable belt in this case (the bladder is not shown). The belt is attached to the conventional clothing element 108 with attachment means 114, shown as button snaps 700, at an anchor point 813, which aids in the cinching down of the strap 814. If the conventional clothing element 108 were tight fitting, the non-connected portion 810 809, shown as section A in FIG. 8E, would not bunch, but instead would stretch. Because the compression means 105 is non-stretch and connected along the connected portion 809 (shown as sections B & C) via attachment means 114, the connected portion 809 is similarly non-stretch. Therefore, substantially all of the adjustment for limb girths is preferably taken up by the non-connected portion 810 809, section C. The coverage of the non-connected portion 810 may range from 10% (substantially disconnected version) to 90% (substantially connected version) of the full nominal circumference of the correlating section of the conventional clothing element 108 where the compression means 105 is to be placed. The precise ranges may be dependent on the material properties of the conventional clothing element 108, and whether a cutout profile 1504 is also provided as in FIG. 15A,B, but the reader shall understand that where the conventional clothing element 108 is more flexible, a smaller percentage of the overall circumference may be disconnected to still cover a wider range of limb sizes. As previously stated, a smaller non-connected portion 810 makes the BFR garment 100 more usable and less hassle with components loose and hanging off. The compression means 105 may then be wrapped around the circumference and secured to itself via an adjustable fastening means 805, shown as a first fastening means 110 disposed along a length and on an outer surface and a section of second fastening means 111 disposed along an inner surface at the disconnected end of the compression means 105. This overlap portion is shown as B in FIG. 8E.

Rather than bunching, in the case of a loose fitting conventional clothing element 108, the conventional clothing element 108 may instead be overlapped with itself to provide a smoother adjustment. Button snaps 700, zippered pleats, or other provisions may be provided shrink a nominal circumference of the garment 100 prior to donning by the user to transform a loose fitting garment 100 into a tight fitting garment 100 depending on the user's limb girth, for subsequent connection of the compression means 105. These variations are not shown for the sake of brevity but tailoring and adjustment of clothing dimensions with buttons, zippers, snaps, etc. shall be understood in the art. Important features of the applicant's invention as illustrated in FIG. 8E therefore are that the compression means 105 and attachment to the garment 100 allows the conventional clothing element 108 to change its dimensions from a nominal circumference around the limb (prior to applying to the limb), and increase or decrease in circumference potentially to accommodate a smaller or larger limb and depending on whether the garment 100 is conventional clothing element 108 is tight or loose fitting.

In FIG. 16A a section of the conventional clothing element 108 is left as an uncovered portion and may therefore stretch, bunch, or overlap to accommodate different limb sizes and apply an initial compression when the adjustable fastening means 805 is secured. If tight-fitting, the initial compression may be provided by the stretch in the garment 100 when being donned, and if loose fitting, may be applied by simply securing the adjustable fastening means 805 in an appropriate location around the circumference. The different with FIG. 8E and FIG. 16A is no portion of the compression means 105 is a non-connected portion 810 in FIG. 16A, therefore it may be easier to fabricate and less bulk dangling off. The benefits in manufacturing and mass production are similarly described elsewhere in this application.

### Adjustable Fastening Means 805—

The device 1 and/or garment 100 further comprises an adjustable fastening means 805 to set the compression means 105 to an open position 811 with an adjustable fastening means 805 shown as hook and loop fastener in FIG. 16A and various other figures. In FIG. 16A, a first fastening means 110 shown as loop fastener is disposed on an exposed surface starting at a second end 807 of the compression means 105 and being disposed along part of, or all of the compression means 105 up to the first end 806. An exposed surface in the purpose of this application is any surface that is accessible from the outside of the garment. The first fastening means 110 is further extended off the first end 806 in the form of a tail strap and a section of second fastening means 111, shown as a section of hook fastener, is disposed on the end of the tail strap. The bladder end gap 1603, the area between the first and second end 807, displaces to allow expansion and then the hook may be placed on the loop to lock a maximum circumference in the open position 811. FIG. 16A also shows an optional rectangular section of hook fastener disposed on the compression means 105 at the second end 807, that mates with the tail strap loop fastener to prevent lateral displacement of that end of the compression means 105. Benefits of preventing lateral displacement have been described in prior applications and further in Alternate Embodiment 8. The reader shall note the optional section of hook fastener may be eliminated and the tail strap formed entirely of hook fastener to accomplish the same results. The reader shall further note that if the tail strap (hook or loop) may be connected further back along the compression means 105 and not at the first end 806. This may allow for overlapping of the compression means 105 and great range in size accommodation.

Hook and loop fastener is cost effective, easy to apply, and readily available. Hook and loop similarly may be non-stretch or may be elastic and therefore provides many advantages as an adjustable fastening means 805. In this application where hook and loop described, the reader shall understand that the two are generally interchangeable if done together. The reader shall also note that where lengths or sections of hook and loop are described, these may be extended, shortened, broken up in to multiple pieces with gaps in between as in FIG. 7E and these variations may be performed to optimize performance and cost of a particular construction.

The open position 811 is the configuration of the compression means 105 where the compression means 105 is fixed in length and location, i.e. not adjusted further, prior to inflation, and the compression means 105 may be subsequently inflated and moved to a closed position where a working compression level is applied to the range of muscles to restrict a flow of blood as desired for proper BFR training. The open position 811 is shown clearly in FIG. 8E where adjustable fastening means 805 (hook and loop fastener) are secured down and no more adjustment or movement of components is needed to initiate inflation in preparation for BFR training session. As described in patent application Ser. No. 15/951,016 Belt Pre-tensioning and Position System for Training a Muscle to Whalen, a stretch factor in the open position 811, which is desired to be substantially non-stretch is preferably at least 40 lb/in but may be configured to work if at least 20 lb/in. As described in prior applications the compression means 105 may be inflated to a closed position, and further moved to a working position by the range of muscles, but such operation has been described extensively in prior applications is referenced herein in its entirety. The working compression level is the compression applied throughout the training session in either the closed position, working position, or a transitory position in between due to contraction of the underlying musculature. The working compression level is therefore correlated with a pressure in the compression means 105 during the training period, the pressure synchronously changing between the closed and working position as the muscles are contracted.

The desired pressure to apply the working compression level may be known prior to inflating the compression means 105, for example a pressure from a previous session or a predictive algorithm, or the pressure may be determined while the compression means 105 is on the user, for example by inflating temporarily to full occlusion of the blood flow into the extremity and then reducing the compression level a certain amount from that “occlusion pressure”, for example to 80% of the occlusion pressure. Since proper and safe blood flow restriction training does not involve fully occluding blood flow in an extremity, the pressure associated with the working compression level may be inadequate as to fully occlude a flow of blood in an extremity during the intended training period.

The adjustable fastening means 805 therefore may be any suitable means such as snaps, cam locks, ladder locks as shown in FIG. 14B, a fabric loop 132 with fold back style belt with hook and loop fastener as shown in FIG. 14A, ropes with slide locks 803 as shown in 13A,B and 8C, D, or any other suitable means for securing a position of the compression means 105 in the open position 811 to prepare for inflation.

The adjustable fastening means 805 is in communication with the garment 100, but not necessarily the compression means 105. As shown in FIG. 13A, B, the adjustable fastening means 805 may comprise a non-stretch fold up flap 701, that folds up along fold line 1205 and encompasses the compression means 105 to limit external expansion, but may not itself be connected or attached to the compression means 105. Conversely, the adjustable fastening means 805 may be directly in communication with the compression means 105 as in FIG. 16A where the adjustable fastening means 805 comprises loop fastener disposed on an outer surface of the compression means 105 starting at a second end 807, and mating hook fastener disposed on the compression means 105 starting at a second end 807 and connectable to the loop fastener.

In relation to a tight fitting garment 100, the tight fitting garment 100 may stretch to accommodate a user as previously described and the adjustable fastening means 805 may lock an outer circumference of the compression means 105 to prevent further expansion. In the case of a loose fitting garment 100, the conventional clothing element 108 may reduce in length by bunching, folding and overlapping, or as otherwise described, and the adjustable fastening means 805 may secure the garment 100 in a smaller circumference and snug to the user's body to apply a desired initial compression level in the open position 811. The adjustable fastening means 805 may therefore prevent unfolding or un-bunching of material.

Herein, the reader shall understand that the components with which the adjustable fastening means 805 communicates may vary and may be modified to optimize the performance of the design and not limited to the specific description and figures disclosed herein.

### Inflation Means—

The device 1 may incorporate an inflation means 106, and the inflation means 106 may be any mechanism or combination of actuators such as valves, pumps, etc, automatic or manual, which supplies and removes pressurized gas to the compression means 105 if the compression means 105 is inflatable. The inflation means 106 could for example be, but is not limited to, an electromechanical pump, or simply a squeeze ball hand pump and may or may not incorporate a release valve, one-way valve, or pressure control valve. As various inflation/deflation circuits have been discussed extensively in the prior art to the applicant, for the purpose of this specification, inflation means 106 shall be understood to also comprise means for removing pressure form the compression means 105. Examples may be that an electromechanical pump itself comprise a pressure relief means or means of maintain pressure, or that a solenoid pressure relief valve is provided for venting gas separate from the electromechanical pump. One or more other components such as one-way valves may be provided to prevent back flow through the air circuit such that the inflation means 106 is not continually working and draining batter power from the energy storage means 905. If the air pathway 119 connecting an electromechanical pump to the compression means 105 comprises a self-acting one way valve in line for example, a second air pathway 119 may be provided on the opposite side of the one-way valve from the electromechanical pump in order to automatically vent the pressurized gas from the compression means 105. The reader shall understand there are many combinations of these systems, many of which have been disclosed by the applicant in prior applications and all such variations are considered within the scope of this application. Further, the reader shall note that the applicant considers all combination of inflation and deflation mechanisms, and accompanying air pathway 119 circuit variations to be one concept termed herein as simply the inflation means 106.

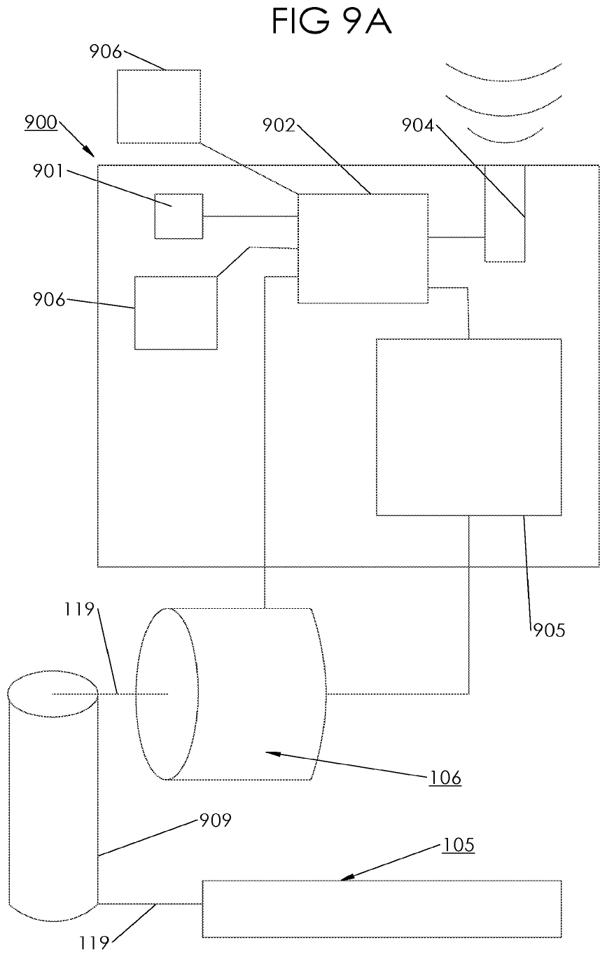

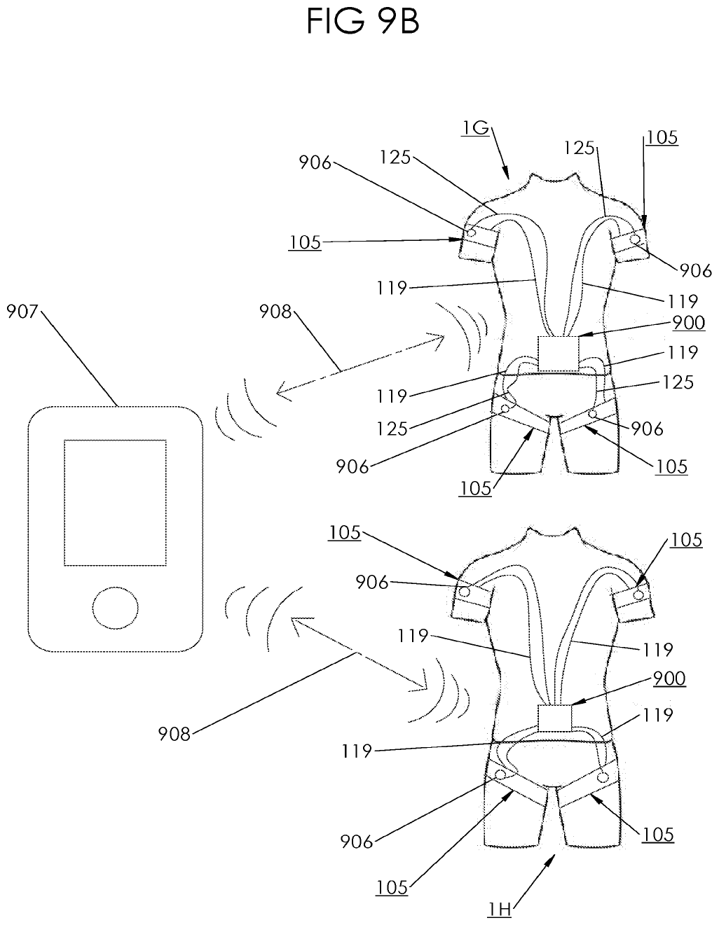

Inflation means 106 may be integrated with the control system 900 or may be a separate item altogether. The inflation means 106 may reside on the compression means 105, attachment means 114, conventional clothing element 108, or it may reside separate from all other components in the BFR garment. The inflation means 106 is preferably controllable by the control system 900 and comprise part of the control system 900 or is located inside an enclosure (not shown) of the control system 900. Alternatively, the inflation means 106 may be an external pump in communication with the control system 900 via an electrical conduit 125 and otherwise separated. The inflation means 106 may be controllable electronically or alternatively manually controlled as in a hand pump with release valve like in a palm sphygmomanometer. The inflation means 106 may be connected to one or more air pathways 119 for transporting pressurized gas to the compression means 105. The inflation means 106 may be in communication with an optional external reservoir 909 shown in FIG. 9A, of compressed air that is used as a storage medium for inflation later without activation of the inflation means 106 thereby allowing inflation of the compression means 105 without simultaneous activation of the inflation means 106. The inflation means 106 may therein be a low flow high pressure pump, and the external reservoir 909 may store gas at a pressure higher than the maximum pressure intended for user with the garment. One advantage to this is that the pump may therefore be smaller, quieter, and lower cost if it doesn't need to provide high flow rates. If an external reservoir 909 is used as the principle supply to the compression means 105, a release valve and pressure sensor may also be in communication with the air pathway 119 to the compression means 105 such that a pressure control system 900 is created for maintaining a desired pressure in the compression means 105 at a lower pressure than stored in the external reservoir 909. A further benefit of the external reservoir 909 is it may allow the pump to operate at convenient periods during the day. If the garment 100 is used for example during a workday, it may be annoying if the pump is making noise during a meeting for example. Therein, a noise sensor may be in communication with the control system 900 to only allow the pump to operate when an ambient noise value is above a certain threshold, for example, but not limited to, the noise produced by the pump. Thereby, even if the control system 900 wants to apply pressure to the BFR garment 100 in a quiet situation, no significant noise is generated and the operation of the BFR garment 100 is essentially imperceptible everyone but the user. The external reservoir 909 may also be useful in times when no battery power or electrical power is available in the case the inflation means 106 is electromechanical.

One or more connection means 107 may be disposed on the inflation means 106 such as a tube, hose barb, or other suitable substantially airtight compression mechanism for transporting pressurized gas to the air pathway 119 or compression means 105 directly. The inflation means 106 may further be interpreted to include a pressure relief means that reduces a pressure in the compression means 105. In this way, the component that creates the pressurized gas (mechanical or electromechanical) may be connected to a reservoir and not actually controlled by the control system 900. The inflation means 106 could constitute a flow control valve that is opened by the control system 900 to allow pressurized gas from the reservoir to fill up the compression means 105 until a target pressure is reached. An additional valve could similarly be provided, optionally controlled by the control system 900, to release pressurized gas from the compression means 105. In this way, the inflation means 106 could be considered to be the valve or valves that let pressurized gas in and out of the compression means 105.

As stated, in the case the inflation means 106 is electrometrical it may be connected to the control system 900 via one or more electrical conduits 125 carrying power and/or control signals as shown in FIG. 9A. The inflation means 106 in FIG. 9A is shown to reside external to an enclosure (not shown) of the control system 900, for example it may be placed on the compression means 105, and receive power from an energy storage means 905, for example a battery, and receives a control signal from a procession means, for example a microcontroller.

### Processing/Sensing Means 906—

The device 1G,H may further comprise one or more sensing means 906 as in FIG. 9B, and sensing means 906 have similarly been discussed extensively in prior applications to the applicant and all references incorporated herein including, but not limited to, heart rate variability sensors, pressure sensors, haptic sensors for gathering user feedback, accelerometers or IMUs, EMG, or EKG sensors among other options. Sensing means 906 may reside at any location on the BFR garment 100, for example under the compression means 105 or adjacent to the compression means 105, or may reside external to the BFR garment 100, or underneath the BFR garment 100, for example a conventional heart rate chest strap. The device 1G,H may also comprise a processing means 902 and the sensing means 906 may be in direct connection with the processing means 902 as shown in FIG. 9A or may rely on wireless communication with the processing means 902. The sensing means 906 may transport data 908 between the sensing means 906 and the processing means 902 which may tell the processing means 902 something about the state of the user, the state of the user's program, or the state of the compression means 105 among other things. The reader shall refer to the applicant's prior application on Efficacy Feedback for further reference as to the type of data 908 and what its uses may be. The applicant will subsequently describe below further novel and inventive uses for data 908 to make the user's BFR training even more useful and easy to perform and work in concert with the concept of the BFR garment 100 as a BFR training tool for the average person to achieve gains and benefit without additional effort as well as for monitoring and control by a coach, healthcare professional, or other kind of instructor.

The processing means 902 is shown in FIG. 9A by a microcontroller, may control the automation of the control system 900 for control of the compressive force on the range of muscles. In the case of a pneumatic system this may include controlling a pressure in the compression means 105 via control of the inflation means 106 and/or a pressure relief valve (not shown). The processor may accept input from sensing means 906 and may run an instruction set according to data 908 stored on the memory storage means 901. The memory storage means 901 may alternatively be combined with the processing means 902 as is common in the field of electronics. The processor therefore serves as the brain of the system and may be pre-programmed with certain protocols or control algorithms, and these programs may be altered by the user or automatically, and may be adjusted before, during, or after a BFR training session. Input from sensing means 906 may influence one or more instructions generated by the processing means 902, such as when to turn an inflation means 106 on and off. If a mechanical cinching mechanism is used to apply compressive force, for example a winch-like mechanism or Nitnol fabric with electricity applied, then the processing means 902 may for example control the actuation of the winching and or application of voltage to the Nitnol fabric until a certain tension or surface pressure is achieved, and this tension or surface pressure data 908 may be communicated to the processing means 902 by a sensing means 906. The processing means 902 may be integrated with the control system 900 or may reside on an external controller 907 as in FIG. 9B, or both. In the case the processor resides elsewhere, a communication means 904 may be provided that relays data 908 and control signals between the BFR garment's control system 900 and the external processing means 902 that is controlling the behavior of the control system 900. The reader shall therefore note that there are many different electrical and mechanical combinations, locations, or otherwise general configurations that accomplish the goals of controlling the BFR garment 100, either directly or remotely, and optionally getting relevant data 908 on and off and all such combinations shall be construed to be within the scope of this invention.

### Control System 900—

The device 1 may further comprise a control system 900 and the control system 900, depicted in FIG. 9A,B in one variation, represents a combination of sensors, actuators such as inflation means 106 or valves (not shown), energy storage means 905, processing means 902, memory storage means 901, etc. which may be combined in whole, or in part to provide core functionality for automated control the BFR garment. While the device 1 may be operated without the addition of control means, or the control means may be detachable from the device 1 or garment 100, the control means may assist the usability of the device 1 by automating functionality and relaying important data 908 about the user's training session or state in general in order to improve training parameters or alert external coaches or medical professionals so they may intervene or make training suggestions. The data 908 gathered by the sensing means 906, and relaying of this data 908 to an external controller 907 for example is described in the applicant's prior application on Efficacy Feedback and these concepts shall be referenced herein. The control system 900 in the applicant's current invention may be integrated with the BFR garment 100, or preferably is a standalone object that may be connected to the various air pathways 119 and/or electrical conduits 125 to communicate electrical signals, and or pressured gas to the sensing means 906, inflation means 106 (if external), or compression means 105 among other things. Various attachment means 114 have been described above for how to connect the control system 900 to the device 1 or BFR garment 100 in a variety of convenient and unobtrusive or discrete ways, such that the control system 900 may travel with a user throughout the day without causing notice by outside observers, or may be worn during a sporting activity for example without interfering with movement of the user.

The control system 900 may house one or all of the above mentioned components and the mechanical location and integrated housing concept is not required, but rather the user shall understand the control system 900 conceptualizes the combination of such similar components in order to make a system that ultimately controls a compression level in the compression means 105 and may or may not relay data 908 about the compression means 105 and/or user to another source or external controller 907. Further the reader shall understand that various components of the control system 900 may reside in different physical locations without departing from the scope of the invention. For example, the inflation means 106, release valves (not shown), etc. may reside in a separate enclosure and be connectable with the rest of the control system 900 components as needed for proper operation. In another variation, a sensing means 906 may be left on a user, while the rest of the control system 900 detached and the sensing means 906 having its own communication means 904 for relaying data 908 to the control system 900. As such, FIG. 9A shows the control system comprising a processing means 902 in communication with: a communication means 904, two sensing means 904 where one sensing means is external to the enclosure, an energy storage means 905 in the enclosure, and a memory storage means in the enclosure. The inflation means 106 further is in communication with the control system but is shown external to the enclosure. However, as stated, the inflation means 106 may also reside in the enclosure. FIG. 9A shows an optional external reservoir 909 in communication with the inflation means 106 and the external reservoir is also in communication with a compression means 105. In this configuration a valve (not shown) would be controllable by the control system to allow air flow from the reservoir into the compression means until the working compression level is reached and then the valve shut. The external reservoir 909 may be omitted and the inflation means in direct communication with the compression means as one variation and the reader shall understand there are many ways to move components around, or change the order of connection, or omit altogether while still maintaining the spirit of the control system 900, which is to provide, maintain, and remove a pressure in the compression means 105 at a minimum.

### Preferred Embodiment—Operation

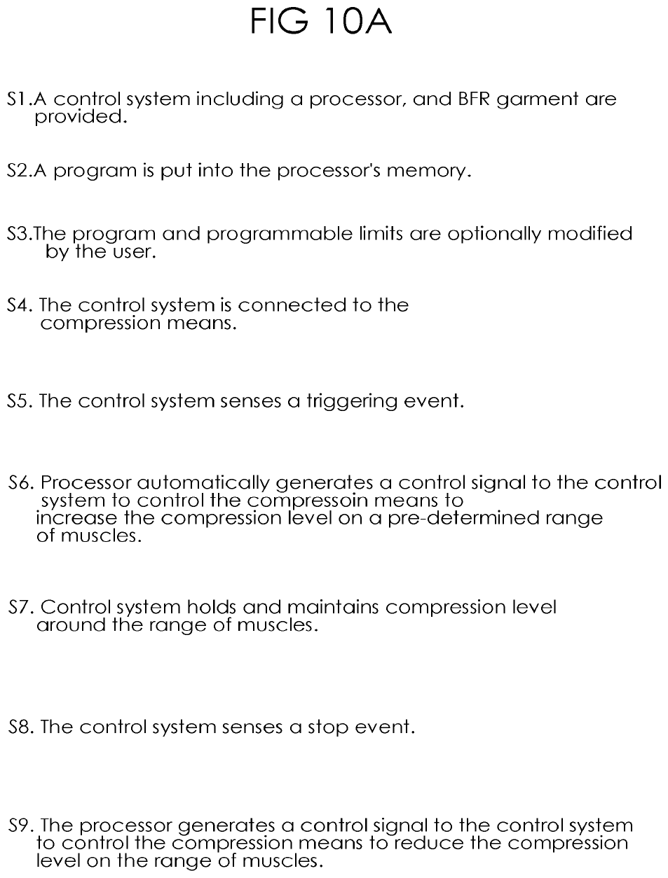

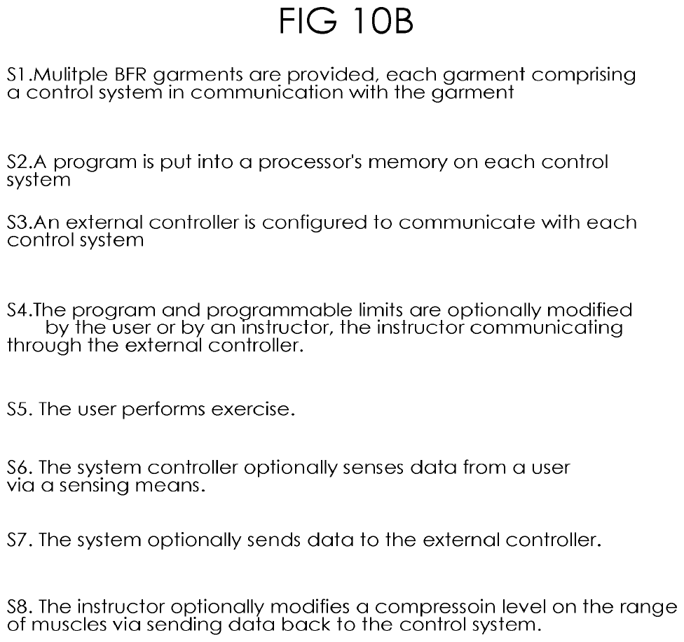

A device 1 in communication with a garment 100 for modifying blood flow has been described in detail, and further variations further described in alternate embodiments, as to how they may be constructed and the myriad of combinations that may lead to an effective apparatus thereof. An important aspect to the utility of the BFR garment 100 is also how it is used, how automation is integrated, and is discussed below and two variations detailed out in FIG. 10A,B.

One primary use of the applicant's invention is in the general training and conditioning of average individuals who are not used to or accustomed to training, or simply don't have time in their day to break routine. The applicant's invention of the device 1, with or without the garment 100, solves this issue by allowing them to do BFR training during their normal day without altering their behavior and can transform a normal daily activity that is not an intentional exercise session, into a session of substantial exercise. The reader shall note the applicant prefers the combination of the device 1 with the BFR garment 100, but shall understand the clothing aspects of the garment 100 are not necessary in all cases and embodiments for accomplish the benefits discussed herein. Research has recently shown that adults using BFR training on their lower body doing walking training (walking around) for six weeks showed gains in normal functional daily activities. This same concept applies to those suffering from metabolic syndrome or other sedentary lifestyle or sarcopenia related issues. They simply put the BFR garment 100 on in the morning and go about their daily activities and the garment 100 does all the work. This mechanism is augmented by the applicants invention and use of automation and sensing means 906 whereby programs may be stored in the processing means 902 or memory storage means 901 of FIG. 9A that describe how the device 1 is to behave during the day and how it should alter its behavior due to the user's activity at any given moment, or minimum or maximum limits on training time throughout the day.

FIG. 10A illustrates a simple example. For example it has been experienced that BFR training in the morning and in the evening can help with sleeping and recovery. Therefore a program can be stored in memory in step 2 of FIG. 10A, and optionally modified in step 3, that from the time the device 1 or garment 100 is put on and the control system connected in step 4, a BFR training period is initiated for a set period of time, and at a period 6 hrs, 10 hrs, etc later a second BFR training period is initiated for a set period of time. The initiation, or triggering event in step 5, may start at a specific time of day, could be programmed by the user or modified in step 3, or could be driven off a sensing means 906 that evaluates a user's alertness. Therefore a time in the day can be a trigger that is pre-programmed and pre-determined. Alternatively, an action by the user as a triggering event in step 5, or data 908 from a sensing means 906 can trigger a BFR training session. For example every time a user starts to walk around, a sensing means 906, for example an accelerometer or EKG o EMG sensor, may relay data 908 to the processor to initiate the compression means 105 and start a BFR training session. The control system then starts in step 6 to apply pressure into the compression means 105 until the desired working compression level is reached. The control system will maintain this pressure in step 7, or maintain a modified pressure if the pressure is changed during the working period. The sensing means 906, or timer as stated above may similarly convey to the processing means 902 when the user has stopped moving, or when a time period is over in step 8, and the processing means 902 may control the compression means 105 to remove the compressive force and restore normal biological conditions in step 9. If programmed in the instruction set, the processor may instruct the control system 900 to remove the working compression level a certain period of time after the user has stopped moving as well, or example between 5-30 min after movement stops. In either of these cases the user may be free to change or toggle parameters to suit their own preferences and styles as type of manual override. The reader shall note therefore there are many signals or conditions that may trigger a control system 900 to apply or remove the working compression level and all such combinations are considered within the scope of the applicant's invention. Similarly modifications or the program, or setting of limits, such as maximum time under blood flow restriction in a day are numerous and considered within the scope of this invention.

BFR training has also been observed to produce a sense of alertness and pain relief, which is hypothesized to occur from a release of endorphins and adrenaline associated with the training. Therefore if a user has an important meeting or competition coming up they may schedule a BFR training session to occur just before that event to improve their performance during the presentation, meeting, or competition. If a user is in pain, they may instruct the device 1 to activate and perform a pain relief BFR training session and this may be suitable simply by continuing to do what they are currently doing whether that is typing on a computer, walking through the mall, etc. The pain relief BFR session may alternatively be automatically triggered by a sensing means 906 that looks at a stress level of the user or otherwise determines if the user is in pain, or may be pre-determined as to occur at one or more points during the day. The BFR training may also be used to improve strength of a repetitive motion, for example a working on an assembly line where the control system 900 and sense are configured to automatically perform a BFR training session or may sense when the user is doing a repetitive motion and then start the BFR training session. Doing a BFR training session during the repetitive motion may be advantageous in that it takes advantage of the concept of specificity of movement and getting stronger doing a specific task. Alternative an instructor, or foreman in the case of an assembly line worker, may control the BFR training via an external controller 907.

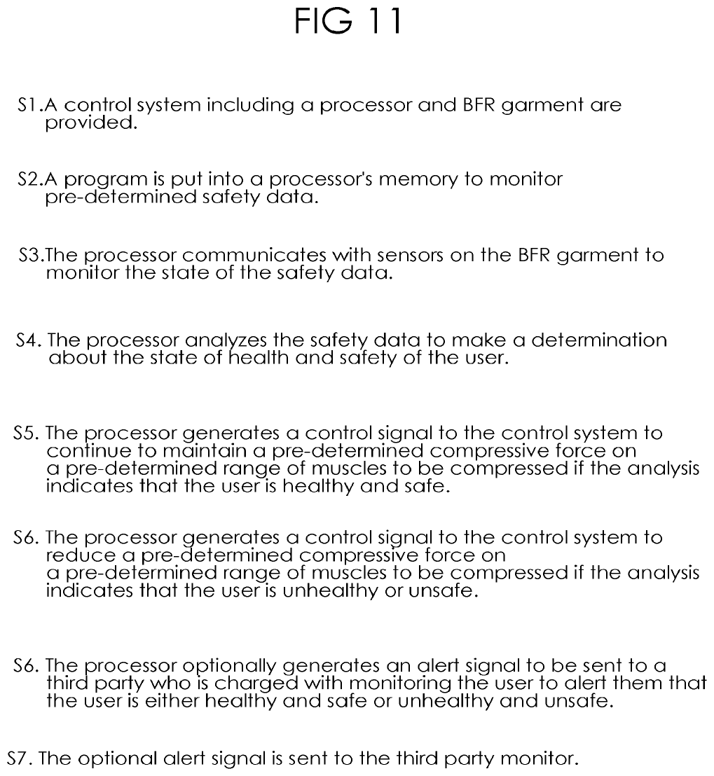

The device 1 may be controlled for safety as shown in FIG. 11 as well whereby a device is provided in step 1. Limits are set in the control system 900 either by the user, pre-programmed, or by a coach or trainer as to the maximum compression allowed, the duration of any bout of BFR training, and the amount of BFR training done in a day, and more in step 2. The reader shall understand there are many such variables associated with a training session such as rep counts, load levels, etc and any variable involved in a training session may be modified by the user, an instructor, or automatically driven by an instruction set in the processing means 902. The reader shall also note many types of data 908 and their use in modifying variables of a BFR training session are described by the applicant's application Ser. No. 15/653,429, and these concepts shall hereby extend to this application.

Additionally, safety data 908 can be monitored such that pressure is maintained in step 5 such that a user is always kept in a safe condition and the device 1 can monitor and automatically release pressure in step 6 if safety data 908 comes back, is analyzed by the processing means 902 which is monitoring and analyzing data in step 3 and step 4, and it is determined the user is in an unsafe or unhealthy state. Safety data may then be optionally related to a third party, such as a coach or healthcare professional in step 7 for further analysis or action. Safety data 908 may relate to heart rate, presence of a pulse or signs of cardiac arrest, since of severe fatigue or inactivity, dehydration or lack of movement as but a few examples. Safety may also relate to EMG signals or EKG signals which may be altered by BFR exercise and where lower or higher levels are generated than are expected so that the device 1 can determine for example that the user is using too high of loads during the training, not taking long enough rest periods, or otherwise doing the training improperly. As with efficacy data 908, this safety data 908 can be relayed to a control panel or external controller 907 so that a monitoring person can take action. The monitoring may continue past the end of a BFR training session as well and may last for up to 60 min so that an instructor or health care professional may have visibility that a user is ok following an exercise session.

Aside from an individual's standalone use of the device 1 and/or garment 100, it may also be used in group training activity as described in FIG. 10B where multiple devices 1 and garments 100 are provided in step 1 for multiple users. Group training is a popular mechanism whereby multiple users are training simultaneously and the communal effect helps push each one harder and make the training more effective and enjoyable. To start a group training session a program is optional put into memory storage means 901 on each control system for each device in step 2. Alternatively the instructor may run a live session with no pre-programmed values. An external controller 907 is preferred then connected with each control system 900 in step 3 so a single controller can be used to affect and modify or read data 908 from each device. The instructor may then optionally modify the program, or any limits such as maximum heart rate, maximum pressure, etc in step 4. The group training session then begins in step 5. During the session each control system 900 may sense data 908 in step 6 about the user and relay this to the instructor via the external controller 907 such as heart rate, HRV, efficacy level, pressure value, etc in step 7. The instructor may then modify a setting of the control system 900, for example the working compression level for a specific user based on the data. Therein the applicant has disclosed a system for managing a group training session with multiple devices 1 and garments 100.

A device 1 that may control the conditions of the training activity and make inflation, setup, movement, communication of data 908 to a coach or instructor via an external controller 907 as shown in FIG. 9B may be advantageous and facilitate the use of BFR in a group training setting. An external controller 907 may communicate with one or more system controllers to control, check, and adjust the status of each participant according to the instructor so that each individual is pushed to their maximum limit and kept in a safe environment. In a group training environment, group training can also be virtual where not all users are in the same location. In any group training environment it may be advantageous to handicap users relative to one another for evening out a competitive field of users who may be at different levels of strength and fitness. In this case, the amount of compressive force may be increased for users who are in better shape in order to make the training activity harder and bring them to the level of their less conditioned counterparts. This handicapping may be combined with the concepts in the applicant's Efficacy Feedback application as well to monitor, tune, and adjust either during an existing session or between subsequent sessions. For example if a group of users is doing a spinning class, those users who are advanced and in very good condition may get the same cycling program but get added BFR pressure in order to make the workout and effort more comparable to the other users who are also spinning the same program. The instructor, or user, may similarly want to modify the working compression level during a session. For example, a working compression level may initially be pre-determined to be 200 mm Hg for the user's arms. However when the user is training, the instructor may see, in particular via heart rate data 908, that the work is too hard or two easy, and may subsequently instruct the control system 900 to increase or decrease the working compression level by changing the pressure in the compression means 105 through the control system 900 directly or via the external controller 907.

Another method of use is for a coach working with one or more athletes wherein a coach may view data 908 about an athlete's condition on an external controller 907 similar to FIG. 9B. In this case the coach may decide an athlete is not working hard enough and send a signal over the communication means 904 to that athlete's control system 900 to apply BFR and increase the workload for that athlete to motivate them to work harder. If the control system 900 is detached from the device 1 and/or garment 100, the coach may also ask the athlete to attach to the control system 900 and then apply a new working compression level to the athlete's device 1 via the control system 900 and/or external controller 907. As mentioned previously the control system 900 need not be attached to the device 1 or garment 100 100% of the time. Similarly a coach may handicap a training routine where athletes in good shape get the compression means 105 activated as the others start to drop off so that the training for them becomes effectively harder. A coach may view data 908 from the device 1 to monitor when an athlete has had an effective session or not and such concepts are detailed further in the applicants prior application on Efficacy Feedback.

A device 1 incorporating a control system 900 and inflation means 106 may automatically activate to apply compression via an external sensing means 906 as well, for example a GPS or accelerometer sensor on a smartphone. The GPS or accelerometer sensor may sense the user is moving or walking, communicate this information to the processing means 902 of the control system 900, and if the conditions programmed into the processing means 902 are met, the control system 900 activates the inflation means 106 (or release valve on an external reservoir 909) and BFR garment 100 is activated. The GPS sensor may also be used for example to sense when the user is in proximity of other users of a device 1 and activate the compression means 105 in an effort to start an impromptu group or partner workout session. The speed and simplicity of BFR training makes it conducive to small “micro-training sessions” throughout the day.

If an optional external reservoir 909 is provided, an instruction set in processing means 902 may be written to activate the inflation means 106 when the external reservoir 909 is below a certain pressure level. This pressure level may be communicated to the processing means 902 via a pressure sensor or other means known to those skilled in the art of pressure control systems 900. The inflation means 106 in this configuration may not be in communication directly, or through an air pathway 119, with any compression means 105. The inflation means 106 may further be activated only during specific conditions, for example a time of day or when an ambient noise level is above the noise generated by the compression means 105 for example. In this way the device 1 and/or garment 100 can stay inconspicuous. The control system 900 may then control the working compression level in the compression means 105 by releasing pressurized gas from the external reservoir 909 into the compression means 105 to the desired level as monitored by a pressure sensor or equivalent means. If the external reservoir 909 is large enough and at a high enough pressure, it may only need to be inflated once in the morning for example, and supply pressure to the garment 100 for the entire day. This may be advantageous in that the inflation means 106 can be disconnected and the main bulk, power requirements, and noise generator removed from the system for the day.

If multiple compression means 105 are in communication with one another via an air pathway junction 123 for example, the air pathway junction 123 may reduce the pressure spikes. As a the range of muscles under a compression means 105 contract and expand, a certain volume inside the compression means 105 is displaced and this leads to a pressure spike according to the ideal gas law that p1×(v1/v2)=p2. So we can see that if two compression means 105 are connected, the ratio of the volumes is what drives the new pressure value. Given the contraction of the range of muscles produces a fixed amount of volume decrease (V2 goes down), the small this volume decrease relative to the overall volume in the system, the more the ratio of v1/v2 goes to 1 and the more P1 approximates P2. An external reservoir 909 may be provided in this case as well, simply as a means to dampen the effects of pressure spikes from muscle contractions.

### Alternate Embodiment—#1—Liner

FIG. 3A (device 1C) and FIG. 3B (device 1D) show a garment 100 adapted to form a liner 115. Garment 100s 100 with liner 115s are generally known in the art and common to see in sporting wear like running shorts, soccer shorts, etc. and may be designed to take the place of underwear. A liner 115 integrated into a garment 100, where the compression means 105 is in communication with the liner 115, may provide a benefit that the BFR aspect of the garment 100 is hidden from public view. As the applicant discusses elsewhere in this application, there are uses where BFR may provide benefits when simply worn during normal daily activity. In the applicant's invention herein described, automation, sensing, and a garment 100 for BFR may make the concept practical for implementation in daily use from the time someone wakes up until the time they go to bed. The convenience is what makes this system and concept possible, however there is still a subset of people who are embarrassed or just don't like to stand out because they are wearing BFR equipment. In an athletic setting, this may be less of a problem, but some athletes may still want to be discreet about their use of BFR. In a normal daily setting however, for example an office worker, they may want to enjoy the benefits of BFR training during the workday without walking around displaying belts, or straps or other things over their shirts that wrinkle and generally draw attention to themselves. A liner 115, where the device 1C,D and compression means 105 is either integrally formed, or otherwise attachable via attachment means 114 as described above, may conceal the fact that the user is wearing BFR equipment and give a normal daily appearance.

For the upper body, the liner 115 may be constructed as a secondary sleeve 116 sewn or otherwise connected to the conventional clothing element 108 such that the main sleeve 116 of the conventional clothing element 108 covers at least a portion of the liner 115. The main sleeve 116 may cover all or only a portion of the BFR equipment, such as compression means 105 as well. It is not necessary for the concept of a liner 115, that the full BFR equipment be hidden although it may be advantageous to be fully concealed. Air pathways, if used, may be similarly concealed on an internal surface of the garment 100, or even sandwiched between a second liner 115, or internal sleeve 116 on the conventional clothing element 108. The air pathway 119 may be passed through the liner 115 to either the internal surface of the conventional clothing element 108 or the external surface. A compression means 105 may be integrally formed on the liner 115 and preferably located underneath the main conventional clothing element 108, or the compression means 105 may be attached via suitable attachment means 114. The compression means 105 and connection means 107, if provided, may be substantially low profile such that they do not create excess bulging under the main conventional clothing element 108, and give the appearance that the user is wearing something. The applicant's prior invention of a barrel inflatable belt, which shrinks in circumference during inflation, is ideally suited as the profile is reduced with inflation vs expanded with inflation like a traditional bladder. FIG. 3A shows an integrated device 1C and garment 100 with the liner 115 concept implemented where one side comprises a compression means 105 integrally formed with the liner 115 and the other side comprises an attachment means 114 in communication with the liner 115. The location of the attachment means 114 or integrally formed compression means 105 is substantially similar and follows the guidelines described previously such at it is easily located properly over the desired range of muscles to be compressed without special steps required by the user for adjusting location.

For the lower body, FIG. 3B illustrates a liner 115 integrally formed with a skirt (shorts or pants would be done in a similar fashion) as the conventional clothing element 108. The liner 115 shown is similar to the shorts of FIG. 2A, and is preferably tight fitting and serve to replace a need to wear underwear. The liner 115 as shown is integrally formed with the device 1D and compression means 105, but as stated above, this is not required. Similar to the upper body garment 100, the air pathways. 119, if provided, may be integrally formed or separable, and may run along the inside surface or outside surface of the liner 115, and may protrude up the top for connection to an inflation source or control system 900. The skirt covers preferably substantially all of the liner 115 and BFR equipment such that it is imperceptible to an outside observer that the user is wearing compression means 105.

In general, it is preferable that the liner 115 is tight fitting to the skin, like a compression garment 100, but is not necessarily so and a loose fitting liner 115 may also be used. The reader shall note that while the liner 115 has been depicted in FIG. 3A, B in relation to a short sleeved shirt and a skirt, the concept of the liner 115 may be extended to any type of conventional clothing without departing form the scope of this invention. The reader shall also note that effectively the liner 115 serves as the garment 100 for the device 1C,D where a secondary layer of clothing is placed over the BFR equipment to conceal it, and this is substantially equivalent to the user simply wearing an integrated garment with device 1C,D and putting another item of clothing on top. The advantages of the liner 115 are that it is integrally formed and does not require the user to wear multiple layers of clothing, which may be too hot or encumber movement more than desired.

### Alternate Embodiment—#2—Integrated Compression Means

The compression means 105 is part of the device 1, and previously has been generalized as being attachable via attachment means 114, but may also be integrated into the conventional clothing element 108 as shown in FIG. 12A, 13 or may alternatively be separately connectable to the conventional clothing garment 100 as previously discussed. In the case the compression means 105 is integrally formed, examples are given in FIGS. 12A,B, 13A,B, 14A,B, 15A,B, 16A-D. The reader shall understand that integral formation of part of the device 1 with the garment 100 shall be considered to integrally form the device 1 with the garment 100 in the context herein.

FIG. 12A shows a sectional view of an garment 100 with integrated device 1 and compression means 105. A conventional clothing element 108 is shown as the inner most layer and may have the properties and characteristics as described elsewhere in this specification. An inner bladder layer 1201 is integrally formed with the conventional clothing element 108. The integration may be achieved by bonding, gluing, stitching, weaving, laminating, seam taping or otherwise inseparably connecting the inner bladder layer 1201 with the conventional clothing element 108. If the inner bladder layer 1201 is connected around the entire perimeter of the conventional clothing element 108, then the inner bladder layer 1201 preferably, not necessarily, is stretchable as well and is preferably. A suitable material may be a polyurethane or PVC film or other thermoplastic for example. The conventional clothing element 108 may also itself be comprise the inner bladder layer 1201 of the conventional clothing element 108, or a relevant portion is made of substantially airtight material such as neoprene rubber. The inner bladder layer 1201 may be the same circumference as the smallest circumference of the conventional clothing element 108 in this case, or may even be smaller such that a portion of the conventional clothing element 108 is not covered around the circumference by the inner bladder layer 1201, forming a bladder end gap 1603. This uncovered area, or bladder end gap 1603, may act as areas A and B in FIGS. 7E, 16A to allow for additional expansion of the conventional clothing element 108 to accommodate larger limb sizes. If the inner bladder layer 1201 has sufficient elasticity and covers the full circumference, then the inner bladder layer 1201 may also accommodate a sufficiently large range of limb sizes without putting undue compression on the largest limb sizes. The reader shall note this is the case for all such tight fitting garments 100 where they are designed to provide less compression on the smallest limb and will be tighter on larger limbs.

In the case the garment 100 is not tight fitting, the inner bladder layer 1201 may be as small as the length equivalent to the smallest intended limb girth and the excess fabric of the conventional clothing element 108 may be bunched up for small limb sizes as shown in FIG. 8E, as one possible configuration. The inner bladder layer 1201 may also overlap itself during the tensioning process if proper layers and locations of fastening means are provided as discussed elsewhere in this specification. In the previous example where the conventional clothing element 108 is loose fitting therefore, both the conventional clothing element 108 and inner bladder layer 1201 may overlap. The inner bladder layer 1201 may also be non-stretch, but then a portion of the conventional clothing element 108 should allow for accommodation of a range of limb sizes. If the conventional clothing element 108 is tight fitting, a sufficient portion of the conventional clothing element 108 and the inner bladder layer 1201 shall remain disconnected so that this portion of the conventional clothing element 108 can take up the stretch necessary to accommodate larger limb sizes. Contrarily if the garment 100 is not tight fitting, the non-connected portion 810 of the conventional clothing element 108 may bunch up such that the two ends of the connected portion 809 form the smallest designed limb circumference. The reader shall note it is also possible for the two connected portions 809 to overlap each other with proper provision of fastening means but this may not be desirable. This concept is shown as the connected portions 809 B and C in FIG. 8E and the non-connected portion 810 A in FIG. 8E. If the conventional clothing element 108 of FIG. 8E is tight fitting then the non-connected portion 810 will stretch and allow for expansion of the conventional clothing element 108 without putting undue compression on the limb. If the conventional clothing element 108 of FIG. 8E is loose fitting, the conventional clothing element 108 may bunch up when the initial tension is applied to make that section of the conventional clothing element 108 snug against the limb of the user. The point the reader shall recognize is that the applicant's invention may be configured according the characteristics of the underlying conventional clothing element 108, it's dimensions, and the designed range of limb sizes it is meant to accommodate.

An outer bladder layer 1202 may be connected to a portion or all of the outer surface of the inner bladder layer 1201 as shown in FIG. 12A. The connection may only be made around the perimeter as in a conventional blood pressure cuff, or preferably is made in a pattern that allows for shrinkage of the inflatable portion as in the applicant's prior invention of a barrel inflatable belt. In the case of a barrel inflatable belt design, the outer bladder layer 1202 is connected along multiple bladder connection joints 1204 as shown in FIG. 12A, 13A. The inflatable barrel design is further desirable as being integrated into the BFR garment 100 because it reduces the overall profile of the limb and will not show bulging externally when another garment 100 is worn on top. The outer bladder layer 1202 may be stretchable as well as shown in FIG. 12A, or may alternatively be non-stretch and comparable to the outer belt material 102 described above and in prior applications to the applicant. If the outer bladder layer 1202 is stretchable, then prior to securing of the outer barrier layer 1203, the entire bladder may expand to accommodate larger limb sizes, and the width of the compression means 105 may be wider as described previously. In FIG. 12A, the bladder and conventional clothing element 108 may be designed to be tight fitting and expand as larger limb are inserted. The outer barrier layer 1203 of 12A, shown as strap 814, is preferably not stretchable and not connected along a substantial portion of the circumference, thereby allowing this expansion to occur. If the outer bladder layer 1202 is not stretchable, or the conventional clothing element 108 is loose fitting, then the outer bladder layer 1202 may be shorter than the smallest designed circumference, or may have a non-connected portion 810 and provisions to overlap or bunch up itself as was described above in relation to the inner bladder layer 1201, such that stretch and/or bunching of the bladder is achieved for a proper initial tension setting.

If an outer barrier layer 1203 as in FIG. 12A is used, the outer barrier layer 1203 is preferably non-stretch and is designed to limit the outward expansion of the stretchable bladder, if both the inner bladder layer 1201 and outer bladder layer 1202 are themselves stretchable. As mentioned above, the outer bladder layer 1202 and outer barrier layer 1203 may be combined as a single piece as long as sufficient stretch or bunching accommodation via overlapping and/or connected portion 809 and non-connected portion 810 are allowed elsewhere around the circumference since this combination will not accommodate a variety of limb sizes by stretch if the garment 100 is tight fitting, or by bunching/overlapping if the garment 100 is loose fitting. The outer barrier layer 1203 may be connected to the conventional clothing element 108 or the outer bladder layer 1202 at an anchor point 813, and the connection may be done via suitable means such as sewing, welding, heat sealing, bonding, or other means known in the art. The anchor point 813 may be on any point around the circumference or may be chosen such that the outer barrier layer 1203 is easily wrapped around the limb for a specific size of limb, for example the median of the intended range of limb girths. The anchor point 813 may be small in area, for example a line of stitches, or may be over a large range of the circumference. In an overlap style belt, a first fastening means 110 may be disposed on an outer surface of the outer barrier layer 1203 along a sufficient length such that when the outer barrier layer 1203 completes a 360 degree turn, a mating second fastening means 111 disposed on an inner surface of the outer barrier layer 1203 connects with the first fastening means 110 to lock the maximum circumference of the assembly limited by the now-closed outer barrier layer 1203.

The reader shall note that the applicant has described the integrated concept with regards to an overlap style of belt, but the same idea may be adapted to a fold-back style belt with a fabric loop 132 as in FIG. 13A, and similar concepts as the applicant has extensively described in previous applications. All such descriptions are referenced herein in their entirety and shall be understood to be integrate-able with the current invention.

FIG. 13A,B shows another variation of an integrated BFR garment 100 with integrated compression means 105. An inner bladder layer 1201 and outer bladder layer 1202 may be integrated with the conventional BFR garment 100 as discussed above in relation to FIG. 12A and are preferably stretchable and encompass the full circumference as described earlier. However the bladder may also be non-stretch, or a portion thereof made non-stretch, or may simply cover less than the full circumference of the limb with provisions in dimensions and connected and non-connected portion 810 809s as previously discussed. FIG. 13A,B show the preferred compression means 105 construction which is a barrel inflatable belt bladder with multiple inflatable chambers 103 and bladder connection joint 1204 disposed around the circumference. The difference between FIG. 13A,B and FIG. 12A is FIGS. 13A,B comprise of a fold up flap 701 that folds up along fold line 1205 and is secured with adjustable fastening means 805, shown as buttons snaps 700, instead of an outer barrier layer 1203. The fold up flap 701 is preferably non-stretch material and serves the same functions as the outer barrier layer 1203 however it may simply be inverted and folded up along fold line 1205 and snapped into place as shown in FIG. 13A. An additional section of the flap (not shown), may extend outward to cover the gap in the fold up flap 701 shown in FIG. 13A, and the additional section may be then be tightened against the limb to connect both ends of the fold up flap 701 thus securing the outer circumference and prevent expansion of the compression means 105. This extension may comprise a first fastening means 110 with a second fastening means 111 disposed on the matching side of the fold up flap 701 such that once the flap is folded up, the gap may be sufficient closed and covered with the extension (not shown) and the two fastening means secured to each other to lock the maximum circumference. As drawn, FIGS. 13A,B shows flexible member 815 as a draw string concept with the fold up flap 701 in the “up” position where it is covering the bladder and the draw strings are pulled tight to limit the outer circumference of the bladder and fold up flap 701 snug against the user's limb. Draw strings, as described in the tension means bellow, may be useful in limiting the circumference in an easily adjustable way such that the user can remove all compression when desired and can quickly draw the strings closed against an adjustable stop 804 without even looking in order to apply the right level of initial tension. The draw strings may alternatively be rotated 90 degrees and multiple cross patterns used in a lacing concept to cinch the two sides of the fold up flap 701 together. Lacing is commonly known in the art on its construction. The adjustable stop 804 described earlier may also be used to provide an adjustable guide to the user for easily applying initial tension without having to look or measure. A user may simply reach under an over-shirt or even grab the slide lock 803 through an over-shirt and cinch the lacing down until the adjustable stop 804 is reached, thus setting the initial tension properly in preparing for BFR training.

FIG. 14A,B show another variation of an integrated BFR garment. FIG. 14A depicts one example of a flat pattern of one leg of a pair of shorts for doing BFR training. The reader shall note there are many patterns for building conventional clothing element 108s and the applicant's invention is not limited only to the pattern concept of FIG. 14A. In this embodiment, a bladder is created between an inner bladder layer 1201 and an outer bladder layer 1202. As shown, the material of the conventional clothing element 108 is preferably air tight and forms the inner bladder layer 1201, and the material of the outer bladder layer 1202 is non-stretch and also substantially air tight. The outer bladder layer 1202 may be connected to the inner bladder layer 1201 when the layers are flat as shown, which makes assembly simple. The connection may be done by any suitable means such as bonding, gluing, sewing and seam taping, or any other means known in the art. In FIG. 14A the connection is made with sewing along the sewing line shown by the dotted lines. The reader shall note that with sewing, holes are created and a layer of seam tape 1601 or other suitable means shall be used to cover the holes and prevent leakage, but that this layer is not shown on the figure for the sake of clarity. The seam tape 1601 may be disposed on both the inside and outside of the garment 100 as in FIG. 16B. FIG. 16B shows a cross sectional view of one variation and illustrates both the concept of bonding and sewing and seam taping 1601. Stitch line 112 in FIG. 16B is shown on the left side and is covered by seam tape 1601 to maintain a substantially airtight chamber 103 as shown. On the right side the outer barrier layer 1202 is bonded to the conventional clothing element 108 whose surface forms the inner barrier layer 1201. If the outer barrier layer 1202 is substantially non-stretch and the bladder formed is a barrel inflatable belt, then no additional stiffener 1602 may be needed.

The bladder may comprise a means, such as a section of tubing welded into a side wall as shown, for connection of connecting means which in turn may be connectable to an air pathway 119 as described in prior applications and as shown in FIG. 14A. The position of the formation of inflatable chambers 103 may be at any point along the circumference and may be vertically located to line up with the desired location for coving the range of muscles. Preferably, the inflatable chambers 103 are located around the interior area of the range of muscles where the vasculature is located as described previously in the applicant's application for applying targeted compression. However the reader shall understand the location of the inflatable chambers 103 may also be on the outside of the limb, rear side, or front side. On one end of the inflatable chambers 103, a strap is connected with first fastening means 110 and second fastening means 111 disposed on an outer surface, and on the other end a fabric loop 132 is connected to the inflatable chambers 103. This configuration illustrates a fold-back style of belt and functions substantially similar to the applicant's prior applications for barrel inflatable belts. The strap is fed through the fabric loop 132 and folded back to apply the desired initial tension and the first fastening means 110 and second fastening means 111 are connected to lock the maximum outer circumference in the open position 811.

FIG. 14B shows a variation on the configuration of 14A wherein the inflatable chambers 103 are formed out of two pieces of substantially non-stretch material and connected to the bottom of a stretchable conventional clothing element 108 (i.e. the shorts leg) around a portion of the perimeter. The connection may be as in FIG. 14A and is shown as a stitch line 112. However in this case the stitch line 112 is not puncturing the inflatable section and therefore no seam taping is required. The flat pattern may comprise a webbing 1503 which is intended to provide a protection for the skin in the area under the tension means provided where there is a gap between the two ends of the inflatable chambers 103. The webbing 1503 may simply be an extension of the standard conventional clothing element 108 and similarly stretch or bunch, or may be a separate piece of fabric or suitable material. Not shown, additional sections of fabric may be sewn to the other side of the inflatable chambers 103 that is not sewn as shown in FIG. 14B. In this way, the section of the BFR garment 100 that must provide compression on the range of muscles may therefore constitute an intermediate section between two elements of the conventional clothing element 108. A tension means may be provided for prescribing a suitable initial tension and may be a ladder lock style lock connected to one side of the inflatable chambers 103 and a strap connected to the other side of the inflatable chambers 103 for feeding through the ladder lock. The ladder lock and strap are illustrated to provide one more example of the myriad of possibilities for applying a suitable initial tension and subsequent locking of this position to constitute the open position 811. The strap is preferably non-stretch such that maximum circumference is locked when the strap is locked in the ladder lock (or other fastening means is activated to lock the initial tension). The strap or ladder lock in FIGS. 14A,B may be disposed at an end of the compression means 105 as shown or disposed elsewhere along the length of the compression means 105. A benefit of disposing along the length is that a portion of the bladder may then overlap instead trying to pull through the fabric loop 132 or ladder lock and this overlap provides a wider range of limb sizes to be accommodated. In the case illustrated in 14A for example, the compression means 105 is attached along the entire perimeter so the compression means 105 cannot physically overlap itself.

FIGS. 15A,B similarly show designs of compression means 105 attached to the garment 100 as integrated, but may be configured to be removable using other concepts in this application. FIGS. 15A,B are described in more detail in Alternate Embodiments 7 & 8.

FIG. 16B-D show cross sectional views of integrated compression means 105 where the conventional clothing element 108 forms the inner bladder layer 1201. In FIG. 16B, the left attachment means 114 is shown as a stitch line 112 connecting the outer bladder layer 1202 to the conventional clothing element 108. The stitch line 112 may be non-stretch or elastic depending on other elements of the design such as whether an outer barrier layer 1203, as shown in FIG. 16D, will be added or not. Where stitching is used, the stitching shall be sealed substantially airtight with seam tape 1601 or an equivalent means. The right side of the chamber shows the attachment being a bonding, welding or other operation that joins the inner and outer bladder layer 1202 together. The reader shall note that the attachment means 114 used is preferably the same for the full perimeter of the bladder and that FIGS. 16B-D show different options used for illustrational purposes only. In the case where additional compression limiting is designed, or if the bladder outer layer is elastic for example, a stiffener 1602 may be used as shown in FIG. 16C. The stiffener 1602 may reside in a channel external to the bladder and may be enclosed by a first fastening means 110 that is used in setting the open position 811 as in other embodiments described herein. The first fastening means 110 in this case may be attached to the outer bladder layer 1202 via similar means as described in relation to FIG. 16B and seam tape 1601 or equivalent sealing methods may be used when stitching is the method of attachment. Alternatively to, or in combination with a stiffener 1602, an outer barrier layer 1203 may be attached to the garment 100 via suitable attachment means 114 such as stitching or bonding as shown in FIG. 16D. The outer barrier layer 1203 may further serve to limit expansion of the bladder radially inward. The outer barrier layer 1203 and stiffener 1602 may be used together by taking first fastening means 110 in FIG. 16C and expanding it to connect to the garment 100 as in FIG. 16D, the outer barrier layer 1203 wrapping at least one full time around the extremity.

The reader shall therefore understand that the compression means 105, whether separable and attachable to the conventional clothing element 108, or integrally formed with the conventional clothing element 108, provides the means of compressing the range of muscles to a pre-determined working compression level. The compression means 105 is designed to accommodate a range of muscles to be compressed, and is designed adjustable enough to not overly restrict flow when the BFR garment 100 is not supposed to be active. To accomplish this, the specific implementation and design of the compression means 105, attachment means 114, and conventional clothing element 108 all work together and may mix and match ideas disclosed or referenced herein as long as the objections of the overall system are met.

### Alternate Embodiment—#3—Foot Pump

The applicant has disclosed so far that the inflation means 106 is either a manual or electromechanical source for pressurized gas. Another embodiment for a manual inflation means 106, aside from a hand pump, is a foot pump which may be worn inside of a shoe and comprise part of the device 1. The foot pump may be formed in the shape of an insole for placement inside of one or both of a user's shoes or may be integrated into the shoe itself. Alternatively the foot pump may be placed underneath a user's shoe in the form of a sole as well. The foot pump may not only provided pressurized gas to the system but may also serve to cushion the user's footsteps during walking exercise.

The foot pump may be actuated by the bodyweight of the user during walking around and this may occur during a specific training time or generally throughout the day. The foot pump may be connected directly to an air pathway 119 via a connection means 107, or integrated directly with the air pathway. An external reservoir 909 may comprise part of the device 1 and the foot pump may incorporate the external reservoir 909 or an external reservoir 909 may be in communication with the air pathway 119 separate from the foot pump. The external reservoir 909 is optional, but if present, it may store pressurized gas which in turn is supplied to the compression means 105 per a user's or control system 900's command to apply a working compression level on the range of muscles. The external reservoir 909 has is described elsewhere in this application and all features shall apply in conjunction with a foot pump. The foot pump therefore may incorporate a one-way valve in line with the air pathway 119 such that the foot pump only can pump air into the external reservoir 909 and air cannot escape back into the chamber of the foot pump.

One or more stop cocks, or on/off valves may be disposed between the air pathway 119 and external reservoir 909, between air pathway 119 the compression means 105, and/or the external reservoir and the compression means 105 such that gas is passed to the compression means 105 when the valves are configured appropriately. All such combinations of shutoff valves, air pathways 119 and external reservoirs 909 shall be considered within the scope of this invention. A bleeder valve may further be in communication with the compression means 105 to bleed air out when the training session is completed. This bleeder valve may also be a pressure relief valve that automatically limits a pressure in the compression means 105 to a pre-determined level and may also be actuated to remove pressure entirely. The series of foot pump, one way valve, optional external reservoir 909, one or more on/off valves, bleeder valve, and/or pressure relief valve therefore forms a fully user configurable manual system that may be configured to supply a pre-determined pressure to the compression means 105 and subsequently apply a pre-determined compressive level to the range of muscles.

The valves mentioned above may be manually operated, for example by rotating a valve handle 90 degrees in the case of a stop cock, or may be automated and connected to a control system 900. The design of the air circuit therefore shall be such that air may be supplied to a compression means 105, a pre-determined pressure may be maintained in the compression means 105 for a period of time, and pressure may be removed from the compression means 105 when desired. The foot pump may be constructed from a bellows like structure or simply an air bladder where the weight of the user stepping on the bladder or bellows causes and amount of air to be compressed and pushed past the one-way valve into the air circuit that will at some point supply the pressurized gas to the compression means 105. A restoring actuator, for example a spring, or material property of the foot pump itself restores the bladder to its nominal shape and size after each step. In this way the bladder can continuously pump air into the system with each step. The external reservoir 909, as has been described earlier, can store compressed gas such that when the user, or control system 900 if the BFR garment 100 is automated, wants to activate the BFR functionality, the external reservoir supplies the gas and no further actuation of the actuator is actually needed. This is beneficial that if the user is in a place where they can't move around to activate a manual pump, and don't want the noise of an electromechanical pump, the pressurized gas may still be supplied for discrete BFR training via the external reservoir 909 and valve system. While the foot pump is not drawn in a figure, it shall be understand that many forms of foot pumps or manual pumps are well known in the art and may be adapted in construction to work with the applicant's invention. A bellows placed inside a shoe for example may constitute a foot pump, as can any other bladder or air bag system that is designed to compress and force a volume of air out an exit, and refill the volume via an inlet. As such are is very commonly understood, the applicant has not redrawn this incarnation of the foot pump as inflation means 106 in this application.

### Alternate Embodiment—#4—Initial Tension Means

FIGS. 8A-E shows various optional initial tension means 800 concepts for applying an initial tension to the compression means 105 for exerting an initial compression level on the range of muscles.

FIGS. 8A-E illustrate several concepts for integrating an initial tension means 800 into the device 1 and/or garment 100 for proper tensioning prior to closing of the compression means 105 to the open position 811 with an adjustable fastening means 805, which is the position of the compression means 105 from which the working compressive level is generated via inflation.

FIG. 8A depicts a conventional clothing element 108 with a pair of button snaps 700 located vertically along one face as an attachment means 114 for attaching the device 1 as a compression means 105. The button snaps 700 may be any similar fastening means such as hook and loop fastener as previously described so long as they secure one end of the compression means 105. The compression means 105 has a mating fastening means, button snaps 700 in the case of FIG. 8A, such that one end of the compression means 105 may be connected to the conventional clothing means and hold its position. The compression means 105 has a first fastening means 110 disposed on its outer surface along at least a portion of its length, and a second fastening means 111 disposed on an inner surface at the end opposite the button snaps 700. The compression means 105 further has an initial tension means 800 in the form of an elastic member 802 disposed at a point along its length and running parallel and towards the end of the compression means 105 with the second fastening means 111 as shown in FIG. 8A. Using an elastic member 802 to apply pre-tension, and its variations, have been described extensively in prior applications to the applicant and are referenced in full regarding this specification. Similarly, while an overlap style of belt is depicted in FIG. 8A, the reader shall understand that a fold back style of belt and its associated initial tension means 800 as described in prior applications may similarly be adapted for connection to a conventional clothing means as in FIG. 8A. The elastic member 802 may be stretched until the second fastening means 111 disposed on the end of the elastic member 802 reaches a pre-determined marking guide 801, or other signified location disposed on the opposite end of the compression means 105 as shown in FIG. 8A. Upon securing the second fastening means 111 to the first fastening means 110 at the desired marking guide 801, the initial tension and circumference of the compression means 105 is set in a repeatable and pre-determinable way. The remaining loose portion of the compression means 105 can then be laid down flat to form the open position 811 and the device 1 and garment 100 are set up for inflation and applying the working compressive level to the range of muscles.

FIG. 8B is similar to FIG. 8A except that the elastic member 802 is omitted. In this case, the compression means 105 is simply connected to the conventional clothing element 108 with the fastening means, shown as button snaps 700, and wrapped around the limb until the second fastening means 111 reaches the appropriate marking guide 801. The reader shall understand that one benefit of connecting the compression means 105 to the conventional clothing means with an attachment means 114, is that the compression means 105 is held in place. This is important for example in wrapping the compression means 105 around the limb particularly on the arms where only one hand is available. The marking guide 801 in this case may be sufficient for wrapping the compression means 105 to the correct initial tension since the conventional clothing means may sufficiently resist rotation of the compression means 105 around the body when applying the initial tension. In this way the initial tension is applied by securing the compression means 105 in the open position 811 without an intermediate pre-tension step as described in the prior applications.

FIG. 8C-D shows an alternative version of an initial tension means 800 wherein the initial tension means 800 can easily and quickly be set and reset. In FIG. 8C, a first end 806 of a compression means 105 is connected to the conventional clothing element 108 with an appropriate attachment means 114 as has been described extensively in this application. Alternatively the compression means 105 may be integrally formed or permanently attached as also described herein. In FIG. 8C, the attachment means 114 is depicted as a piece of hook and loop fastener disposed around a portion of the circumference of the conventional clothing element 108. The reader may note the attachment means 114 may also be a stitch line 112 that permanently connects the compression means 105 to the garment 100. A pair of flexible members 815 are disposed on a top edge and bottom edge of the compression means 105 on a second end 807, and the flexible members 815 may be a strap, or rope, or any other suitable member for cinching two ends of the compression means 105 together. The flexible members 815 are routed through openings 808 on the first end 806 of the compression means 105 and the openings may simply be holes in the material itself, grommets, or the equivalent. Adjustable stops 804 are disposed along each of the flexible members 815 in between the first end 806 and the second end 807 of the compression means 105. The position of the stops may be moved but shall correspond to a pre-determined initial tension that is desired. Adjustable fastening means 805, depicted as slide locks 803 in FIG. 8C,D are disposed along the flexible members 815 on the opposite side of the openings 808 from the adjustable stops 804. Slide locks 803, or their equivalent, are generally known in the art in use with draw strings and other cinching type mechanisms. The initial tension means 800 of FIG. 8C therefore functions as follows. The user places the adjustable stops 804 at a point along the flexible member 815 corresponding to a pre-determined circumference around the range of muscles corresponding to a desired initial tension. This may be prescribed via markings on the flexible member 815, or may be calibrated by the user based on feel and then the adjustable stops 804 locked down on the flexible member 815 and not movable until the user wants to move them. The locked position of the adjustable stops 804 dictate the minimum circumference of the compression means 105 prior to inflation and therefore set the initial tension of the compression means 105. The user further may then either loosen the compression to a completely loose position where no effective compression is provided to the user other than what is designed by the conventional clothing element 108 if it is tight fitting, or the user may cinch down the slide locks 803 and pull the first end 806 and second end 807 of the compression means 105 towards each other to sandwich the opening 808 between the adjustable stop 804 and the slide lock 803. By adjusting between the completely loose position and the initial tension position simply by moving the slide lock 803 the user can easily adjust the initial compressive force from zero to the desired initial compressive force in preparation for doing BFR training. Additionally, as the slide locks 803 may not be able to carry the high tensile forces that develop during inflation and BFR training, additional fastening means (not shown) may be added to lock the first end 806 and second end 807 of the compression means 105 together to prevent further expansion. Additional fastening means may be in the form of a hook and loop fastener pair, hooks, or other means as has been extensively covered in this application. Alternatively the slide locks 803, a cam or ratchet mechanism may be used in conjunction with the adjustable stops 804. Cams and ratchets are commonly known in the art of cinching down strapping, and all such variations of this concept may be applied and understand that where an adjustable stop 804 is used, the cam or ratchet mechanism may cinch the strapping up to the point of the adjustable stop 804 and no further. In this way, the reader shall understand that the general concept is one of cinching between a loosened configuration and initial tension position and all mechanisms or means of cinching that accomplish this shall be considered within the scope of this invention.

FIG. 8D illustrates one variation on this concept where an adjustable fastening means 805 is disposed along the length of the compression means 105 and connected via suitable attachment means 114, shown as hook and loop fastener in FIG. 8D. Where as in FIG. 8C, the compression means 105 is not allowed to overlap, the illustration of FIG. 8D is designed to overlap. The reader shall understand that the design of 8C may be modified to allow overlap by moving connection point 812 of the flexible member 815 back from the second end 807 toward the first end 806, thereby allowing the portion between the connection point 812 and the second end 807 to overlap with the first end 806. In FIG. 8D, the adjustable fastening means 805 may be movable along the length of the compression means 105 to allow significant overlap. Attachment means 114 are shown as button snaps 700 in this case and serve as the anchor point 813 for the compression means 105 thereby holding the compression means 105 in place from rotating as the user cinches down. The initial tension means 800 of FIG. 8D otherwise operates substantially similar to that of FIG. 8C.

The reader shall also note that instead of flexible members 815 and adjustable stops 804, a mechanism using a winch or cinch strap may be used to pull two points of the compression means 105 toward each other. An initial tensioning means 800 such as the brand Boa Fit (https://www.boafitcom/products) would suite this purpose for tightening to the extremity and taking up any slack, particularly in the case of a loose fitting garment 100. The reader shall understand that such a connector is also a flexible member 815, the applicant is just providing another alternative for adjusting the length of the flexible member as it pertains to tightening the compression means 105 to the user to apply the desired initial tension in the open position. Such connectors may be advantageous that, like FIG. 8B, they provide constitute both the initial tension means 800 and the adjustable fastening means 805.

Finally the reader shall note that the initial tension may also be applied to the compression means 105 by virtue of an elastic property of the compression means 105 itself vs the elasticity of the conventional clothing element 108. While an elastic compression means 105 has other considerations required to apply sufficient compression as discussed herein, it also has advantages of in terms of initial compression. The elasticity of the initial compression means 105 as in FIG. 13A,B, or the garment 100 itself as in FIG. 8F,G or FIG. 16A applies initial compression due to the stretch in the fabric itself in the case of a tight fitting garment. In FIG. 13 A,B the initial compression means 105 is made of elastic material and therefore stretches with the conventional clothing element 108 over the user's limb before being wrapped and secured with the adjustable fastening means 805. The convenience of this is no action is required by the user to set up or adjust anything, they simply put the garment 100 on, which is one design goal of the applicant's invention. In the case of FIG. 8F,G the cutout profiles 1504, and potentially sections of the conventional clothing means that are left available to stretch, displace to accommodate larger limb sizes. This displacement necessarily exerts a reaction for on the user to apply the initial compression to the range of muscles.

FIG. 12B shows another example of an initial tension means 800 in the form of an integrated device 1 and garment 100 with elastic, substantially airtight compression means 105 in form of a barrel inflatable belt, formed around the circumference by joining to the conventional clothing element 108. In FIG. 12B, the conventional clothing element 108 comprises the inner bladder layer 1201 as well as described in Alternate Embodiment 2 for how to integrate the compression means 105 into the conventional clothing element 108. The preference with FIG. 12B is that the garment 100 is tight-fitting and that the elasticity of the garment 100 and compression means 105 accommodates a wide range of limb sizes. An outer barrier layer 1203 is shown connecting two points of the garment 100 with adjustable fastening means 805 shown as a first fastening means 110 and second fastening means 111 in the form of hooks. The outer barrier layer 1203 is preferable non-stretch and so by connecting these two points, the outer barrier layer 1203 is limiting the maximum displacement between the two points, effectively transforming this section of the compression means 105 and garment 100 to be non-stretch. The more of the compression means 105 and garment 100 that is covered by the outer barrier layer 1203, the more initial compression will be applied for a given limb size because there is less of the circumference that is elastic.

The benefit is as follows. The outer barrier layer 1203 is appropriately placed for a given target extremity size. The garment 100 is donned by the user and the garment 100 stretches to accommodate the extremity size and the stretching applies an initial compression to the extremity based on how much of the compression means 105 is covered by the outer barrier layer 1203. If the extremity size is small, it may be advantageous to cover a larger portion of the compression means 105 with the outer barrier layer 1203 to apply a greater initial compression. Conversely, if the extremity is large, a smaller portion may be connected by the outer barrier layer 1203 as the garment 100 and compression means 105 need to stretch more without applying undue initial compression. The exact compression and position may be adjustable by the user and determined based on limb size, intensity level, or other factors previously described by the applicant for setting appropriate initial compression levels.

However, in relation to FIG. 12B, the compression means 105 and garment 100 may also be loose fitting and a portion of the garment 100 folded and overlapping itself to reduce the circumference, and the outer barrier layer 1203 connecting two sections of the garment 100 with adjustable attachment means 114 to secure the garment 100 in the shrunken state and against the extremity. In this case the shrunken state is applying the initial compression and securing the open position 811. The compression means 105 may further be mildly elastic, at least 20 lb/in or non-stretch and the configuration can still work.

### Alternate Embodiment—#5—Compression Means Standalone

The applicant has described extensively many different features, benefits, components, and combinations that may be made to form a device 1 and garment 100 for optimizing BFR training. In addition to the inclusion of pumps, air pathways. 119, electrical conduits 125, inflation means 106, control systems 900, etc, a device 1 and garment 100 comprised of just a conventional clothing element 108 and either an integrated or attachable compression means 105 shall also constitute a useful embodiment of the applicant's invention. The compression means 105 in this case may comprise only a connection means 107 for input of the pressurized gas, but the neither the device 1 nor garment 100 need to include provisions for connecting air pathways 119 and/or electrical conduits 125, or integrating those features. In this case the applicant dons the garment 100 and device 1 (compression means 105), or connects the compression means 105 after the garment 100 is put on, and from there may operate the device 1 by connecting, inflating, and disconnecting external air pathways. 119, inflation means 106, and or control systems 900. This is a simplified version of the applicant's invention, but the reader shall note that it does form a basic embodiment of the device 1 plus garment. This alternate embodiment also illustrates how it's possible to remove various elements while still maintaining the spirit of the applications invention to make BFR training simple and easy to do.

### Alternate Embodiment—#6—Connected Compression Means

FIGS. 2B,4 show an alternate embodiment for routing and connecting air pathways 119 between two compression means 105 and a single inflation means 106. The reader shall recognize that there are many different ways to pressurize pneumatic compression means 105 and some of these variations have been discussed previously. It may be desirable however to connect compression means 105 together when the compression means 105 are located around similar ranges of muscles, such as the thigh region and/or the upper arm region. By connecting the compression means 105 together pneumatically the user ensures that during inflation, both compression means 105 are set at the same level and only a single inflation action is required. Additionally, only one inflation means 106 is required instead of two for simultaneous inflation of the compression means 105. This saves on bulk, cost, time, and improves the reliability and comfort of the system overall. While it is possible to connect inflation means 106 around different ranges of muscles, for example one are and one leg, the applicant's preference is to connect similar ranges of muscles in order to maintain more consistent BFR training.

The device 1B may therefore comprise one or more air pathway junctions 123, and the compression means 105 are connected via their own air pathway 119, which are then joined at the air pathway junction 123 as shown in shorts of FIG. 2B, and in the long pants and shirt of FIG. 4. The air pathway junction 123 may be integrally formed by, for example, splitting a tube in a Y configuration or transitioning a single tube into two tubes, or may be a separate piece such as a Y-splitter with hose barbs on each of the three ends. The important aspect of the air pathway junction 123 is that it takes a single input air pathway 119 and splits it into two or more air pathways 119. The reader shall also note that while a split into only two air pathways 119 is shown in FIG. 2B, it is possible and within the scope of this invention to split the air pathway 119 into more than two pieces for connection to more than two compression means 105, or further cascading multiple air pathway junctions 123 in series to “tree-off” the air pathway 119 and supply more compression means 105. The reader shall also understand that the location of the split may be at any point along any of the air pathways 119 as long as the goal of pneumatic connection of compression means 105 is met. FIG. 4 shows two sets of air pathways 119 running parallel up preferably an out-seam 118 of the pants and connecting the inflation means 106 with compression means 105. Each air pathway 119 is split via an air pathway junction 123 into two branches, for example with a Y-splitter with hose barbs, and each branch then goes to its own compression means 105. The air pathways 119 and air pathway junction 123 may be loose running inside of the pants or may be attached to the pants via suitable attachment means 114 as previously described. One set of air pathways 119 is shown terminating near each compression means 105 (one for each leg) and the other air pathway is shown terminating near the waist of the user. Terminating near the waist may be a comfortable location for attaching another garment 100 with two compression means 105 for the arms, therefore not requiring a second control system 900, but rather using one control system located down at the ankle to supply pressurized gas to four different compression means, one for each limb.

Other than the connection of air pathways 119, routing of the combined air pathway 119, and other accommodating modifications, the construction of the rest of the device 1B and/or garment 100 may be substantially similar to the other concepts discussed in this application.

The operation of the system has a unique and distinct benefit in addition to simply reducing the number of inflation means 106, electrical connections, time to setup and associated modifications with this component reduction. During exercise, one of the main goals of the BFR garment 100, or otherwise general compression means 105, as stated in the prior art is to maintain the level of compression as constant as possible. This is discussed in patents to Sato and in implementation of KAATSU and Delfi tourniquet systems which monitor and adjust pressure to keep it constant. A fundamental issue all of these systems have is that the response time of the electromechanical actuators (i.e. pumps and valves), is that they do not react fast enough to accommodate rapid muscle expansion from contraction. Therefore any fast movements, as advocated by BFR training, result in pressure spikes in the compression means 105 and corresponding spikes in compressive force on the range of muscles.

With a pneumatic system, where pressure can adjust at the speed of sound, having a larger reservoir 124 as called out in FIG. 2B, or volume of air, has been discussed in prior applications to the applicant as advantageous for maintaining more constant bladder pressures. This is because with a larger volume of air, the percentage of volume decrease that happens with a muscle contraction, is substantially proportionally smaller, and so the corresponding effect on pressure increase is smaller, corresponding to a lower spike in compressive force on the muscles. Whereas all prior art systems are described, built, and implemented with separate inflation means 106 and compression means 105, and no interconnection of the compression means 105 whatsoever, these systems ignore a simple “free” benefit by connecting the two bladders together. When the bladders in each compression means 105 are inflated initially, the bladders are automatically put at the same pressure level. This makes it easy for a user to be sure they are doing the same thing on both sides and saves operational steps of inflating the two bladders separately. During operation, when both sets of range of muscles are activated, the pressure will increase as it normally would in each belt and there is not a substantial difference between the two compression means 105 being separate pneumatically. However, when imbalances are created between the two limbs, or single limb activities like running are done, when one of the limbs contracts and the volume decreases, some air moves from the contracted limb to the relaxed limb. Because the volume is effectively doubled (because you have two compression means 105), the pressure spike is reduced substantially, potentially in half. So for many scenarios and exercises, there is a great benefit in terms of comfort and safety by having pneumatically connected compression means 105 via an air pathway junction 123 as shown in FIGS. 2B, 4.

### Alternate Embodiment—#7—Slit & Weaving Clothing Element

The conventional clothing element 108 may comprise one or more cutout profiles 1504 around the range of muscles to be compressed as shown in FIGS. 8F,G and FIG. 15A,B. Cutout profiles 1504 may allow for additional expansion or ease of overlapping portions of the conventional clothing element 108 to accommodate a greater range of sizes.

While the conventional clothing element 108 itself might be elastic, it is generally advantageous that the portion of the compression means 105 or garment 100 around the range of muscles is not elastic in order to apply sufficient compression. This has been extensively discussed in prior art applications to the applicant, the importance of a non-stretch outer layer. However if a circumferential section of the garment 100 is non-stretch, the section being either the conventional clothing element 108 itself or combined with the compression means 105, then this section cannot be used to accommodate a wide range of muscles to apply proper compression, so these two desired properties work against one another. The applicant's invention of one or more cutout profiles 1504 solves this problem as the cutout profiles 1504 relieve the circumferential tension in the garment 100 material in the location where the non-stretch portion exists, around the range of muscles.

The cutout profiles 1504 may be as few as 1 cutout profile 1504 as in FIG. 15A,B, or may be up to 20 cutout profiles 1504. In FIG. 8F, the cutout profiles 1504 allow a device 1 (compression means 105) in the form of a belt to weave in and out of the conventional clothing element 108. A first fastening means 110 in the form of loop fastener may be disposed on a portion of the outside surface of the compression means 105 and may be attachable to a second fastening means 111 in the form of a hook fastener disposed on an inner surface of the conventional clothing element 108. A series of first fastening means 110 may be further disposed on an outer surface of the conventional clothing element 108 and a second fastening means 111 disposed on an inner face of the compression means 105, the two being attachable to lock an option position of the compression means 105.

To operate, the compression means 105 is attached to the inside surface of the conventional clothing element 108. This connection serves as an anchor point 813 to prevent the compression means 105 from moving substantially relative to the conventional clothing element 108 during tensioning. The compression means 105 is then woven in and out of the cutout profiles 1504 around the range of muscles. The user may pull the compression means 105 tight to apply a desired initial tension and in so doing the compression means 105 may shrink in circumference and move slightly relative to the conventional clothing element 108. The compression means 105 is then fastened to the outer surface of the conventional clothing element 108 with the second set of first and second fastening means 111 to lock the open position 811.

The reader shall note that such a construction and method as shown in FIG. 8F,G may be used for both a tight fitting and a loose fitting conventional clothing element 108. In the case of a tight fitting element, the compression means 105, while being preferably non-stretch, is not connected to the conventional clothing element 108 aside from the anchor point 813 and until the end, so the conventional clothing element 108 is allowed to stretch and expand and the width of each cutout profile 1504 will expand in this case. In the case of a loose fitting garment 100, the conventional clothing element 108 will not be affected until the compression means 105 is tensioned, and during this process, any bunching of material will be evenly distributed around the circumference. In addition the portions of the conventional clothing element 108 not covered by the compression means 105 may overlap the covered portions on either edge during the tensioning profess such that no significant bunching is seen at all.

Finally the reader shall note that the cutout profiles 1504 in this case also serve to lock the compression means 105 from displacing laterally along the length of the limb. This is an added benefit in maintaining consistent compression as the applicant has observed that significant compression is lost in an overlapping style compression means 105 if the overlapping portions are not substantially fixed laterally relative to one another.

### Alternate Embodiment—#8—Slit & Webbing 1503 Clothing Element

A device 1 and compression means 105 integrated with a slit garment 1500 of FIG. 15A (device 1I), and FIG. 15B (device 1J) shows the cutout profile 1504 may also be used without weaving a compression means 105 in and out of the conventional clothing element 108. FIG. 15A depicts a single cutout profile 1504 at an end of the conventional clothing element 108 in the form of a pleat, the cutout profile 1504 comprising a first slit edge 1501 and a second slit edge 1502. The conventional clothing element 108 further comprises an optional webbing 1503 that covers the cutout profile 1504 so the user's skin is not exposed to the compression means 105 or adjustable fastening means 805.

FIG. 15B similarly shows a cutout profile 1504 in the form of a barbell wherein a slit is formed substantially over the range of muscles, the first slit edge 1501 and second slit edge 1502 being close to one another so there is minimal gap between them. The cutout profile 1504 may be located at any point around the circumference of the range of muscles but is preferably on a front face 121 of lateral face for easy access to the adjustable fastening means 805.

The cutout profile 1504 may be in the form of a triangle or barbell as shown or any other suitable shape. The location of the cutout profile 1504 may be at a bottom edge of the conventional clothing element 108 as in FIG. 15A, or along a length of the conventional clothing element 108 as shown in FIG. 15B.

An adjustable fastening means 805 is preferably disposed to cover the cutout profile 1504 by attaching to the compression means 105, or alternatively the garment 100 (for example the conventional clothing element 108) in proximity to the first slit edge 1501, spanning the cutout profile 1504 and the second slit edge 1502, and connecting to the compression means 105 or alternatively the garment 100 (for example the conventional clothing element 108) at an appropriate location around the circumference to set a desired initial tension. The adjustable fastening means 805 is depicted as hook and loop fastener in FIGS. 15A,B, but the design may be adapted to use any kind of adjustable fastening means 805 as herein described.

FIG. 15A shows one variation of adjustable fastening means 805 where first fastening means 110 in the form of loop fastener is attached (either permanently or removably) to a first end 806 of the compression means 105 and the length is sufficient to cover a maximum limb size, for example 20 cm to 50 cm on arms and 45 cm to 100 cm on legs. The first fastening means 110 (loop fastener) is also disposed along an outer surface of the compression means 105 around the circumference. A second fastening means 111 (hook fastener) is disposed at the loose end of the first fastening means 110 to be securable to the first fastening means 110 at an appropriate location around the circumference. Additionally, an optional second piece of second fastening means 111 (hook fastener) is disposed at a second end 807 of the compression means 105 to mate with the loose end of the first fastening means 110. The advantage to adding one or more additional pieces of first fastening means 110 to an outer surface of the compression means 105 is that if the limb is small in circumference, a significant portion of the compression means 105 may be overlapped and loose underneath the loose end of the first fastening means 110. There may be a tendency for the compression means 105 to slip out from underneath the overlapping first fastening means 110 and loss of compression may occur. The additional pieces therefore connect the compression means 105 to the first fastening means 110 at multiple points around the circumference to maintain intact compression at all times and prevent lateral displacement of the overlapped and overlapping sections relative to one another. In practice the applicant found this is critical for applying a consistent compression level to the range of muscles.

FIG. 15B shows an alternative to FIG. 15A wherein a second fastening means 111 replaces the first fastening means 110 loose end and the additional pieces of second fastening means 111 are omitted. In this scenario, the second fastening means 111 will be in connected to the first fastening along the entire overlapping portion and no relative lateral displacement is possible where the compression means 105 can slip out from underneath.

In a tight fitting conventional clothing element 108, the webbing 1503 of FIG. 15A may expand and the first slit edge 1501 and second slit edge 1502 may displace away from one another. The length of the first fastening means 110 loose end is long enough in this case to accommodate the full range of designed limb sizes. For example in an arm garment 100 with minimum designed circumference of 25 cm, the conventional clothing element 108 would have a designed circumference of less than 25 cm so that some initial compression is applied in all cases. For a leg garment 100 the designed circumference may be less than 45 cm so that limbs greater than 45 cm always experience some compression.

In a loose fitting conventional clothing element 108, the webbing 1503 of FIG. 15A may do one of two things. In one case, the webbing 1503 may bunch up underneath the loose end of the first fastening means 110 as the initial tension is applied as in FIG. 8E. Additionally, the first slit edge 1501 may displace and overlap the webbing 1503 and second slit edge 1502, the webbing 1503 also overlapping itself and the second slit edge 1502 in this scenario. As the webbing 1503 is folding and overlapping itself, any bunching may be reduced and the user comfort improved. Whereas only allowing bunching of the webbing 1503 (or conventional clothing element 108 for that matter as in FIG. 16A) may allow adjustment of a certain amount, allowing overlapping may approximately double the amount of adjustment allowed, and therefore may also be more advantageous in requiring fewer sizes of loose fitting garments. Opposite to the above, for a loose fitting arm garment 100 where maximum limb size may be 50 cm, the circumference of the garment 100 would be greater than 50 cm, and similarly for a leg of 100 cm, the designed limb circumference of the garment 100 is greater than 100 cm.

The reader shall further note that a garment 100 and device 1 may be designed to act as both loose fitting and tight fitting. For example, in relation to FIG. 15A, if the arm range were 25 cm to 50 cm, the garment 100 circumference may be 37.5 cm nominally, the width of the webbing 1503 may be 6.25 cm so that the first slit edge 1501 and second slit edge 1502 may displace toward each other and overlap the full width of the webbing 1503 of to apply the desired initial compression to arms down to 25 cm, and first slit edge 1501 and second slit edge 1502 may displace away from one another, the webbing 1503 stretching 100% to accommodate arms up to 50 cm. In this way, the garment 100 and device 1 may minimize the range of sizes or bulk and extra material experienced by users at one end of the size spectrum.

### Alternate Embodiment #9—No Clothing Element

The reader shall note that while the garment 100 aspect provides many unique advantages, the concept of automated use and control throughout the day may be applied to a control system 900 and compression means 105 that may be independent of any conventional clothing element 108 or garment. As noted at the outset, the device 1 is the core component and may be used standalone, attached to, or integrated with a garment. The applicant's preferred embodiment is use of the device 1 with a garment 100, but the use as a standalone device 1 is described below.

In such a scenario the device 1, comprising compression means 105, or multiple compression means 105 may be donned by the user under a daily garment 100, on top of a daily garment 100, attached to a daily garment 100, on top of or attached to an under garment 100, and all such configurations shall be in the context of this invention, but the daily garment 100 having no special provisions related to the BFR system. Therefore the main difference in this embodiment is the clothing component may be considered separate and non-essential and inventive concepts herein still applicable. A daily garment 100 shall be considered in the family as the applicant has already described in relation to the conventional clothing element 108 such as, but not limited to: shorts, shirts, pants, skirts, business suits, socks, sleeves, jackets, under garments, etc.

The user therefore may wear normal daily clothing independent of the control system 900 and compression means 105 and simply put on the compression means 105 and control system 900 in an appropriate and comfortable manner for wearing throughout the day, similar to the applicant's prior art designs and descriptions.

The operation of this embodiment is substantially similar to the preferred embodiment wherein a control system 900 may sense a movement, data 908, or other physiologic aspect of the user and the movement, or other physiologic aspect, may be related to a part of the user not under blood flow restriction, or may be related to the range of muscles being compressed. The control system 900 may activate the compression means 105 upon sensing an appropriate activation signal, or otherwise determining it is appropriate to do so. For example the control system 900 may wait for the user to be continuously active for more than two minutes before activating the compression means 105, or the control system 900 may wait for a user's heart rate to reach a minimum value, or drop below a maximum value. The reader shall understand there are many triggers that may indicate an appropriate time to automatically apply compression to begin BFR training.

Further, the activation may be based on a time of day, such as when a user should get up and move around and therefore serve as a notification that it is time to get out of a sedentary position to get some exercise. The activation may also come from a GPS signal where the GPS signal is sensing movement of the user, for example from a smartphone, in order to activate the compression means 105 since the system knows the user is moving around. Or, if an external controller 907 is used, a coach, or other instructor may actuate the compression means 105 when they feel it is appropriate. Therefore the control system 900 may transform normal daily activities into BFR training to maximize the efficiency of the user's daily movements and activity.

The control system 900 may maintain a working compression level to the range of muscles as follows. The working compression level may be applied as long as the user is moving or the range of muscles under compression are moving or contracting. The compression level may be applied as long as a certain heart rate is maintained. The working compression level may be maintained until a certain efficacy data 908 has been obtained, the efficacy values being extensively described in prior applications to the applicant. The working compression level may be applied for a set, pre-determined period of time, for example between 5 min and 20 min or in some cases as long as 60 minutes. The working compression level may be applied during set times of the day. Still other criteria may be understood by the user as known to those skilled in the art or described elsewhere in this application or other applications to the applicant.

The control system 900 may then remove the pressure and working compression level when it determines is appropriate. This may be for example, but not limited to, a fixed period of time after starting the compression, when a safety data 908 about a user indicates the user is unsafe or unhealthy, when the movement of the range of muscles or user in general stops, or as otherwise determined it is time to end the BFR training.

Herein the reader shall see how the applicant's invention of the device 1 can be used with the core components of one or more compression means 105 and a control system 900 worn independently or in tandem with daily clothing to maximize the efficiency of normal every day activities. The reader shall further understand that the methods of when to apply, how long to apply, and when to remove a working compression level shall also apply in the case of use with a conventional clothing element 108 of the preferred embodiment and all such concepts in this alternate embodiment may be used in combination with other embodiments described herein.

### Alternate Embodiment—#10 Attachment Means 114

The attachment means 114 as previously described may be part of the device 1 or garment 100 and may connect the device 1 to the garment. FIG. 7A depicts a series of button snaps 700 place circumferentially around the conventional clothing element 108 for attachment of the compression means 105. In this embodiment, the compression means 105 has a mating set of button snaps 700 disposed along its length on one edge, and may have more button snaps 700 than the conventional clothing element 108 for better alignment across different limb sizes as discussed above. FIG. 7A shows the compression means 105 attached to the conventional clothing element 108 via the attachment means 114, in this case button snaps 700. The button snaps 700 alone do not interfere with the conventional clothing element 108 expanding. As the compression means 105 is preferably non-elastic (as discussed later and in prior applications), the button snaps 700 on the compression means 105 may be fixed in distance relative to one another. Thus, when the attachment means 114 is donned by the user, the spacing on the compression means 105 and attachment means 114 may not line up. In this case, not all button snaps 700 need be used, or the portion of the conventional clothing element 108 in between each button snap 700 may be stretched or bunched to accommodate the different in distance. Alternatively, the portion on the compression means 105, or an additional member not shown, may be movable relative to the body of the compression means 105 such that the distance between the button snaps 700 on the compression means 105 relative to one another may be changed. Such a construction may involve having the button snaps 700 attached to a thin “finger” of fabric protruding perpendicular to the body of the compression means 105 and being flexible enough that it can deform and move laterally parallel to the body of the compression means 105 to either move closer or farther way from adjacent button snaps 700 on their own respective fingers coming off perpendicular and coplanar with the compression means 105 body. This is but one example and the reader shall understand the basic concept of FIG. 7A whereby button snaps 700 are used to attach a compression means 105 around a portion of the conventional clothing element 108 in a removable manner and in a way that can be adjustable and comfortably accommodate users of different limb dimensions.

FIG. 7B depicts a fold up flap 701, connected to the conventional clothing element 108 at a bottom edge with a permanent attachment means 114 such as a stretchable stitch, and securable to the clothing element on a top edge with a series of button snaps 700. As shown, the fold up flap 701 may be secured at the bottom edge via sewing, welding, bonding, or other suitable means. Similarly the fold up flap 701 may be secured along the top edge by button snaps 700, zippers, hook and loop fastener, hooks, or any other kind of fastening means known to those skilled in the art. When the fold up flap 701 is connected along the top edge, a channel is formed and the compression means 105 may either be inserted at this point, or may have been previously applied and “folded into” the fold up flap 701 when the top edge was secured. An optional first fastening means 110 may be applied to the conventional clothing element 108 around at least a portion of the circumference that is covered by the fold up flap 701 in the secured state. A mating second fastening means 111 may be applied or integrated into the compression means 105 such that it may be connected to the first fastening means 110 and hold the position of the compression means 105 while the fold up flap 701 is folded up as shown in FIG. 7B. The first fastening means 110 and second fastening means 111 have been covered extensively in prior applications to the applicant and may be, but not limited to, button snaps 700 or hook and loop fastener. The first fastening means 110 may be a short section, for example 1 inch in length and just enough to hold the compression means 105 in place will the fold up flap 701 is secured, or may encompass the full circumference of the conventional clothing element 108 over the range of muscles for example as elastic loop fastener. The first fastening means 110 may be non-stretch or may be stretchable to help accommodated limbs of different girths. The fold up flap 701 therefore may provide additional support to secure the compression means 105 or it may provide only support for the compression means 105 (i.e. if the first fastening means 110 is not provided and the compression means 105 is loose inside the channel formed by the fold up flap 701). In the case the fold up flap 701 does not fully secure the compression means 105 the compression means 105 may loosely slide within the fold up flap 701 channel thereby allowing the conventional clothing element 108 to expand independently of the compression means 105 which may be desirable. The fold up flap 701, when forming a channel may cover a substantial portion of the compression means 105 and therefore may also provide another first fastening means 110 on an outside surface of the fold up flap 701 as shown in FIG. 7B. A second fastening means 111 may be disposed on an underside of the compression means 105 so that when the compression means 105 is wrapped around the limb inside of the channel created by the fold up flap 701, and comes full circle, the compression means 105 overlaps the fold up flap 701 and secures the first fastening means 110 disposed on the outside of the fold up flap 701 to lock the compression means 105 in an open position 811.

The reader shall note, the open position 811 is defined as the position of the compression means 105, just prior to inflation of the compression means 105 to the working position.

The reader shall understand that many different constructions for pneumatic compression means 105 in the forms of belts, straps, bands, both of a fold back nature and of an overlap nature have been described in the prior art and all such constructions and configurations shall be adaptable to this variation of the attachment means 114. The core concept of this attachment means 114 is using a fold up flap 701 to secure a portion of the compression means 105 to the conventional clothing element 108 for further tensioning and applying an appropriate compressive force to the range of muscles. All such modifications, additions of fasteners, etc., which are required for adapting various styles of compression means 105 shall be considered within the scope of this invention.

FIG. 7C shows an alternate embodiment of an attachment means 114 in the form of a sleeve 116 integrally formed with the conventional clothing element 108 wherein a compression means 105 may be fed and inserted. The sleeve 116 does not require additional components like button snaps 700 and may allow for similar benefits of letting the compression means 105 slide freely relative to the conventional clothing element 108 so that when a user puts on the BFR garment 100 originally, the conventional clothing element 108 can accommodate the user's limb girths as designed and subsequently the initial tensioning and working compressive force may be applied to the range of muscles. The sleeve 116 may be integrally formed by bonding, sewing, hooking, or otherwise connecting both a top and bottom edge to the conventional clothing means. The sleeve 116 may cover a substantial portion of the limb, for example up to 100%, in which case it may be desirable that the sleeve 116 and attachment means 114 of the sleeve 116 also be elastic, or the sleeve 116 may cover only a minimal portion of the limb, for example 10% to 30% and may be substantially non-stretch. Such methods of connecting a sleeve 116 to a piece of clothing are well known to those skilled in the art of sewing and shall be applied herein. The sleeve 116 may be a single sleeve 116 or multiple discrete sleeves 116 and the length of each sleeve 116 may vary, but in all cases the compression means 105 shall be sufficiently held in place on the conventional clothing element 108. The width of the sleeve 116 may be wider than the compression means 105 so that the compression means 105 can easily be fed through the sleeve. As in FIG. 7B, the sleeve 116 may comprise a first fastening means 110 (not shown) disposed on an outer surface for securing a compression means 105 as well as an attachment means 114 (not shown) for securing a first end 806 of the compression means 105 such as button snaps 700 in FIG. 8A. The compression means 105 may be inserted into or removed from the sleeve 116 through an opening and the position of the opening and optional attachment means 114 may determine where the compression means 105 is located and how it is oriented. For example the location of the opening may be in a posterior, anterior, or lateral position relative to the user's body for easy access to the opening for insertion of the compression means 105.

FIG. 7D shows an alternate embodiment of an attachment means 114 in the form of one or more loops 127 in communication with the conventional clothing element 108. A loop 127 may be considered to be similar to a belt loop and the compression means 105 may be fed through one or more of the loops. The loop 127 may be any suitable material, such as, but not limited to, the same fabric as the conventional clothing element 108 is made from. The loop 127 may be sufficiently wider enough that the compression means 105 can be easily fed through and may hold the compression means 105 in location for subsequent initial tensioning and applying a working compressive force to the range of muscles. The loops 127 may be spaced sufficiently that the compression means 105 is adequately held in place, or may only be a single loop for holding one end of the compression means 105 while the other end is wrapped around and secured in order to stay in place by friction for example. More than one loop 127 may be advantageous as it may hold the compression means 105 in place during movement from displacing laterally, which can be problematic as described later, or when putting additional clothing over, and prevent the compression means 105 from sliding up or down to an undesirable location.

Displacement of the compression means 105 in generally relative to the garment 100 is undesirable and may happen easily, particularly on the legs where many legs are conical in nature, causing the compression means 105 to want to slide down, and reducing therefore the compression force generated on the legs.

FIG. 7E shows another embodiment of an attachment means 114 in the form of one or more pieces of first fastening means 110 as discs placed periodically and circumferentially around the range of muscles and the discs are integrally formed or connected to the conventional clothing element 108. The first fastening means 110 may be for example a hook fastener and the compression means 105 may have an inner surface, which is a loop fastener. Many such combinations are possible for the first fastening means 110 and second fastening means 111, the point being that the compression means 105 is periodically attached to the conventional clothing means in at least one location around the circumference. By placing the first fastening means 110 periodically, instead of continuously, a non-stretch version of the first fastening means 110 may be used whereby the sections of the conventional clothing element 108 in between the periodic first fastening means 110, denoted by A and B in FIG. 7E, are allowed to stretch and lengthen in the case of a tight fitting garment 100, or bunch or overlap in the case of a loose fitting garment 100, in order to accommodate different limb circumferences, or reduction in limb circumference when initial tension or the working compressive force is applied to the range of muscles. In the configuration of FIG. 7E, the compression means 105 may be laid over the first fastening means 110 and simply stick onto the surface of the conventional clothing element 108 and may then further be tensioned initially and fastened down in the open position 811 as will be described later. The reader shall note that attachment means 114 of FIG. 7E may also be elastic loop fastener disposed around a majority, and potentially all of the circumference, or in other words 50-100% of the circumference of the range of muscles such that elasticity of the loop fastener expands with the conventional clothing element 108. In this way a similar end result to FIG. 7E is obtained. In the case where the compression means 105 is overlapping, another set of first and second fastening means 111 may be disposed on the compression means 105 for fastening the compression means 105 in an open position 811 as described later.

FIGS. 8A,B,D similarly show attachment means 114 as button snaps 700 for securing one end of a compression means 105 to the conventional clothing element 108 in a removable fashion. FIG. 8C shows attachment means 114 as hook and loop fastener for securing a full length of the compression means 105 to the conventional clothing element 108. FIGS. 8A-D further illustrate therefore how either one end, or an entire length of the compression means 105 may be attached to the conventional clothing element 108 without departing from the spirit of this invention.

### DESCRIPTION—CONCLUSION, RAMIFICATIONS, SCOPE

Thus the reader will see that the various inventions described herein provide an economical way to easily create a multifunctional, safe, inexpensive, easy to use blood flow restriction system and BFR garment 100 for incorporation therein. Additionally the reader will see that inventions described herein may take advantage of current mass production processes to keep the additional cost minimal, and that by reducing component count, for example via integrated BFR device 1s and garments, the applicant has not only reduced the manufacturing costs but reduce the level of complexity of operating the system, and reduced the bulk of the system which, since it is a wearable product to be used during exercise, is a significant factor as Sato himself describes. The applicant has further invented a means through which any user at all, whether athletic or deconditioned, may take advantage of the benefits of BFR without changing anything about their normal daily routine, thereby greatly expanding the scope of realistically potential users and possible dramatic impact on the health of the general population thereby.

While the above description contains specificities, these should not be construed as limitations on the scope of the invention, but rather as an exemplification of preferred embodiments thereof. Many other variations are possible.

### Elasticity

Where the term stretchable or elastic is used in this specification, the reader shall note that the applicant may be referring to an elastic property of the construction, and not necessarily that the materials themselves, as individuals or when combined, have elastic properties, i.e. form a material or combinations of materials that themselves stretch in nature. The applicant acknowledges that the materials may have elastic properties themselves, but this may only be an option, not a requirement.

Similarly, the reader shall note that the applicant, when defining materials as inelastic, understands that all materials stretch to some degree when a force is applied. The applicant's description of the term ‘inelastic’ within the context of this application shall therefore be construed to comply with the applicant's intent and purpose for each such element within each embodiment as described. For example, a 200 denier ballistic nylon fabric, coated with polyurethane may stretch 5% when subject to a stretching force, but such material may be considered inelastic or non-stretch in the context of this invention when compared to prior art bladders made of rubber which may stretch up to 100% for the same given applied force.

Materials described similarly may be understood to encompass combinations of materials, varying material properties such as durometer or elastic modulus, lengths and widths, and profiles, which affect properties such as elasticity and coefficient of friction, may be considered within the scope of this invention. Further the readers may note that where a material may be discussed as elastic, a non-elastic material may be combined with an elastic material to form what would be considered the original member (or visa-versa), but which is now two components and may not specifically match the description herein. However, in such cases, the readers may note that the applicant has in fact considered that materials may be combined to perform the function of the elements of the inventions described herein, but has not made all such descriptions because of the endless possible combinations possible. Yet another example is the reader may note that some element properties may be altered to remove various components. For example the inflatable portion of the compression means 105 may have some degree of elasticity in order to compensate for muscle contraction.

Again, the reader may note that all such combinations or omissions of components, or altering of various component properties may be considered within the scope of this invention.

### Bladder Shapes/Sizes

For example, in the case of inflatable bladders formed to provide compression means 105, the inflatable portion may be of any suitable geometry, size and shape to provide sufficient blood flow restriction as discussed above. Bladders may come in multiple lengths and widths to accommodate a range of individuals, and not necessarily minimized in the number of variations, and may match general sizing of the garments 100 or may be denoted as a separate size range altogether, but rather targeted toward a specific range of limb girths, or user types. It may be noted that wider cuffs have been shown to restrict flow to the same extent at lower pressures and may offer more comfort for certain applications that don't require dynamic movements. Such width variations for a specific user, such as assisting the elderly, may improve comfort while maintaining effectiveness. Belt shapes which employ enough tissue displacement to restrict venous return, such as some examples described herein, may be used, and may not necessarily cover the entire limb. All such configurations of profiles, sizes of belts, gas bladders, locations placements of such belts on the body, and bladders on belts, may be considered within the scope of this application.

### Open, Closed, Working Positions

The reader shall further note that the open, closed, and working positions as defined in the applicants prior applications shall apply to this specification. Open position 811 constitute the position at which the initial tension is provided and next action of the compression means 105 is inflation in order to move the compression means 105 to the closed position. The closed position constitutes the position at which the muscle is relaxed but the working compressive force is applied to the range of muscles. The dimensions of the compression means 105 in the positions mentioned above could also constitute the small or large end of any range or spectrum described in this specification. For example, if a belt size range is targeted at limbs of 18 cm-33 cm, the open position 811 could be a closed circumference of any size that fits over at least an 18 cm limb up to at least a 33 cm limb. In case the specified limb is 18 cm for example, the open position 811 may be 18 cm or slightly larger. Similarly the closed position in this case is less than the open position 811 by some amount. The specific amount depends on a variety of factors such as the starting open position 811 and tension on the limb, the amount of blood flow restriction desired, and the amount of pressure applied to reach the desired restriction. Similarly, the working position when wrapped around a limb entails an inner belt circumference that is greater than the closed position nominal circumference, and less than, or up to the nominal open position 811 circumference, thereby illustrating that the inner surface of the belt shrinks when moving from the open position 811 to the closed position, and elongates when moving from the closed position to the open position 811. As in the example above the open position 811 may be 18 cm in circumference, the closed position may be 16 cm in circumference, and maximum working position may be 17 cm in circumference. Or, if more compression is desired the open position 811 may be 18 cm in circumference, the closed position 15 cm in circumference and the working position 17 cm in circumference. Therein, the reader shall understand that these terms may vary considerably depending on a specific situation and the specification and appended claims shall take into account all possible scenarios and interpreted to the broadest extent.

### Combinations of Materials and Design Elements

The reader shall note that many design elements and material property combinations have been discussed and that these factors: attachment means 114, connection means 107, air pathways 119, electrical conduits 125, conventional clothing element 108s, compression means 105, control systems 900, external controller 907s number of inflatable chambers 103, height to width ratio of the chambers, width of the compression means 105, range of limb circumferences to cover, compression means 105 or conventional clothing element 108 material properties, cutout profiles 1504, body interfacing component (not shown), and targeted compression vs full encirclement of the limb, to name a few may all be combined in full or in part, altered in some way, shape, quantity or form, or otherwise modified so as to improve or alter the properties of the inflatable belt. For example, there may be as few as 1 chamber in the case of a targeting inflation belt, o a conventional cuff design, and this chamber may or may not contract and provide desired shrinking effects depending on its dimensions, even though it may not be as effective as having more chambers. Similarly, there may be 50 chambers for full encirclement of a large limb, and still achieve some amount of shrinkage and provide elasticity. The device 1 may be used by itself without consideration for a garment 100 or other clothing, may be attachable to conventional clothing modified appropriately, or may be integrated with a garment. The device 1 may partially reside on the garment 100 and components such as the inflation means 106 attached and detached, or the sensing means 906 may remain attached along with the compression means 105 but the system controller removed. The reader shall understand the intent of the applicants design permutations through the description and figures and apply a broad interpretation in particular to the device 1 and garment 100, or combination of the two. The applicant has covered in this application, the physics, mechanical properties, and tradeoffs of these various important properties and design elements, and the reader shall understand that all such combinations and modifications of these features that affect or improve the properties and function of the inflatable belt for restricting blood flow in a limb, shall be considered within the scope of this invention, and the applicant's invention shall not be limited solely to the combinations depicted in the figures or described in this specification.

### Materials

Various garment 100 designs and control systems 900 and other system components have been described herein, and various material constructions and configurations have likewise been disclosed. Various components being elastic, and relative degrees of elasticity have further been noted. The reader may note that for the sake of brevity, not all such combinations and material types have been discussed, but all such combinations, material properties or configurations may be considered within the scope of this invention. For example, in the case of the fastening means or attachment means 114: cam-locks, ratchets, and hook and loop fasteners have been described or referenced, however many other such means of fastening two objects together may be used such as a high friction joint tri-glide style mechanism, glues or adhesives, ropes or knots, mechanical hooks, buttons, racks and pinions, high friction surfaces, etc may be consider encompassed within the term fastening means and this term interpreted as broadly as possible. Further, in the case of elastic member 802s or fabrics, polyurethane coated fabrics may be substituted for PVC coated fabrics or a similar material, and urethane molds, but may be of latex rubber, or similar material. In all such cases where specific materials are called out, the readers may understand that, this specification is but one example, and as long as the general concept described is achieved, the specific material, or specific property thereof, is not a requirement of the invention.

### User

The user in the context of this application may be deemed to mean the person using the inventions described. This may be a client, patient, instructor, personal user, doctor, athletic trainer, coach, etc.

### General

One skilled in the art will recognize any minor modifications that would be needed for such an intermingling and such modifications may be considered within the scope of this specification and claims. Further, it may be recognized that many of the components described may be combined into a single object via different manufacturing processes such as welding, injection molding, casting, etc. While the applicant discusses some of these options briefly in the application, it may be recognized any and all combinations of the components discussed herein may be considered within the scope of this application and covered by the claims written. Similarly, it may be recognized that many components in the system and their connection points 812, or connection means 107, or anchor point 813s may also be interchanged or rearranged to achieve the same effect as the disclosed configurations. For example, where it is discussed that it may be advantageous to de-couple the inflation means 106 from the compression means 105, and a pressure relief valve is used to limit a maximum pressure in the belt, the pressure relief valve may reside either on the belt side of the coupling or the inflation means 106 side of the coupling. In the case of residing on the belt side of the coupling, then no further shutoff mechanism is necessary on the belt side of the coupling. However, the invention will function substantially the same if the coupling employs a shutoff function to keep air in the belt, which is opened during connection of the inflation means 106, and the pressure relief valve is on the inflation means 106 side of the coupling. In such a case, as long as the inflation means 106 is connected, the pressure relief valve is in the same air-circuit as the belt, and limits the pressure therein. Upon disconnection however the pressure relief valve is not connected in the air-circuit of the belt, however neither is the inflation means 106 and thus there is no risk of too high pressures accumulating in the belt. Thus the system is substantially similar in both cases. This is but one example, and in general, valves, and valve types, fastening means, such as cam locks, hook and loop fasteners, ratchet mechanisms, belt springs, inner and outer belt material 102s etc. may be interchanged, used in quantities of more than one, altered in width, length, or profile, employed in conjunction of overlapping belt styles, or doubling back of belt styles for locking, or more complicated belt designs such as those shown in patents to Sato, and the inventions disclosed herein may be considered to have encompassed all such permutations and combinations of such components. Yet another example is the inflatable belt may have two input port 104s, one to allow air in and another in communication with an outlet system such as a pressure relief valve. While such design is not shown in the figures above, the reader may note this concept is another example of how multiple items may be employed, and components shifted within the system to connect with different components, while the same overall system and effectiveness is maintained. Further still, the location and placement of various elements may be moved and altered such that they appear to differ from the figures shown, and description attached, however, all such configurations and combinations may be considered within the scope of the inventions disclosed herein. For example, in the case of the hook and loop fastener shown on the inflatable belt in FIG. 12A, the hook and loop fastener may be exchanged and the function still maintained. In addition, the location of the input port 104 may be in the middle of the inflatable compression means 105 instead of on one end. A body interface component (not shown) such as neoprene rubber may be permanently attached the inflatable bladder, or it may be removable. If removable, the attachment means 114 may be for example, hook and loop fasteners, and the fasteners may be along the edges or may run along the full width of both the inflatable bladder and body interface component. In the case the hook and loop fasteners run along the full width, they may be elastic such that the inflatable bladder may still inflate against the user's limb. As illustrated, there are many constructional permutations and combinations, and altering of various material properties which yield satisfactory results in a compression means 105 for use in a blood flow restriction system, and all such combinations and permutations and material property choices may be considered within the scope of this invention.

### BFR Garment Configurations

The device 1 and garment 100 configurations described above have been illustrated to be configurable in multiple ways including: integrated with the device 1 and compression means 105, separate from the device 1 and compression means 105, based on loose fitting clothing or tight fitting clothing, used with different kinds of compression means 105, initial tension means 800 and attachment means 114, integrated with electronics, or purely manual, and more. The concept of the device 1 and garment 100 and the effect on making BFR training ubiquitous may employ any of these concepts as stand alone, or may combine aspects of the different embodiments discussed.

For example, where conventional clothing elements 108 have been discussed as related only to an upper or a lower body, a full body suit may instead be used and the compression means 105 integrated into both the lower and upper body similar for example to FIG. 5.

As another example, the inflation means 106 has been described as part of the control system 900 or as a separate mechanism, the inflation means 106 may also be connected and integrated with the compression means 105 so that if the compression means 105 is detachable, so is the inflation means 106. In the inflation means 106 is integrated with the compression means 105, for example an electromechanical pump connected directly to an air bladder around the limb, then no air pathways 119 are needed and the device 1 is simplified.

As has been discussed in both this application and patents to Sato, there are a variety of ways to form a compression means 105 around a user's limb and each has some advantages and disadvantages as discussed in the various applications. The reader may recognize that the inventive concepts disclosed herein may be considered adaptable, by changing, but limited to, the following: size, length, location, neighboring components, adding or removing one or more components, such as a fabric loop 127, material property, such as elasticity, etc. Such modifications represent numerous permutations and configurations which are too many to reasonably depict and describe herein, however the reader may understand that the applicant has thought of such reasonable applications, and may consider as such, part of the scope of this disclosed invention.

### Full BFR Garment Integration—

The device 1 and garment 100 as described in the applicant's invention herein are each formed from a combination (and not necessarily all) of conventional clothing element 108(s), optional attachment means 114, compression means 105, optional initial tension means 800, adjustable fastening means 805, and optional control system 900 and associated sensors or external controller 907s. The integration of two or more of these items provides a device 1 or garment 100 that is wearable by user as part of their daily routine, or as part of a sporting apparel that a user will wear anyway and therefore reduces further barrier to entry or use by incorporating BFR into the normal daily activities of the user. Automation and methods of use as discussed below further increase the utility and make the BFR training even easier, or in fact not requiring any thought by the user while still remaining safe and effective. The applicant's current invention, when combined with prior inventions around compression means 105 and efficacy feedback means further optimize the training methodology of BFR for a wide swatch of users so that the training is comfortable, affordable, easy to do, and requires little to no extra effort from what users ordinarily do, and may be easily monitored and guided by a professional if needed.

The applicant has also disclosed constructions for integration of the device 1 and compression means 105 such that there is even fewer actions required by the user to implement BFR in their routine. By integrating the compression means 105 completely and using constructions and materials that are suitable for washing and normal wear of garments, the applicant provides a superior design and invention for an integrated BFR garment 100 that is adaptable to various limb sizes and provides sufficient compression via pneumatics vs. simple strapping. The applicant has further solved the issues around how to adequately adjust a garment 100 comfortably and efficiently and provide a range of tension setting possibilities that make adjustment of the garment 100 throughout the day easy to do and further increase the comfort and utility of the BFR garment. The applicant has also provided means for combining compression means 105 volumes and making reservoirs 124 that buffer the pressure spikes more than in the prior art with integration concepts joined together with the conventional clothing means to keep air pathways 119 inconspicuous, unnoticeable, and non-obstructive. The applicant has further disclosed how to combine the garment 100 with a device 1 comprising controllers and sensors so that more automation, ease of use, and effective use can be achieved. In total, the applicant has disclosed a system that can perform safe and effective BFR training on a user without the user having to add any additional steps in their daily routine or take any time out of their day they need to dedicate to BFR training.

### Intended Use

It has been observed that the BFR garment 100, and in particular the compression means 105 can be used to shape and alter the physical form of a user's body. In particular compression means 105 placed below the deltoid muscle and above the bicep muscle, when compressed, can actually enlarge the appearance of the bicep muscle. For some people, in particular young men, this “pump effect” may be desirable during situations where a stronger looking body can provide more confidence or other social advantages liking make one more attractive to the opposite sex. On the lower body, it has been observed that compression means 105 placed below the buttocks region has the effect of “lifting” the buttocks and giving a more fit, or sporty physique than otherwise. Other types of clothing garments, in particular leggings and such for women, attempt to firm up or tighten the body to improve physical appearance. The applicant's invention can do the same, and in fact better job for the slimming down and contouring the lower body, and in particular the buttocks region to lift it up. Therefore, in use, not only is the user actually improving physical form, and appearance of physique in a long term sense from the effects of the BFR training, they are also gaining temporary benefits in appearance just from wearing the BFR garment 100 or simply the compression means 105. Further integration into clothing and style is outside the scope of this invention, but the reader shall understand the BFR garment 100 may also be sold or marketed as improving shape or physical appearance of the body.

### Data 908 Types

The reader shall note that many types of data 908 about a user may be collected and used by the control system 900 in analyzing when to apply the working compression level and when to remove the working compression level. Examples, not limiting the scope, are: whether a user is moving, the user's physical location, or physiologic state of the user, a heart rate pulse event, a heart rate variability measurement, EKG values, EMG values, a pressure value, a repetition event, a movement event, a haptic event from the user, or an orientation of a limb. The reader shall understand that appropriate sensors for collecting such data 908 are known in the art and such sensors may be integrated with the applicant's invention as the sensing means 906 where appropriate.

Although the description above contains many specifications, these should not be construed as limiting the scope of the invention but as merely providing illustrations of some of the presently preferred embodiments of this invention. Thus the scope of this invention should be determined by the appended claims and their legal equivalents, rather than by the examples given.

1. An integrated garment and blood flow restriction (BFR) training system, the system comprising:
a garment comprising an elastic fabric;
a first air pathway coupled to a first compression element that is disposed at a first extremity of the garment;
a second air pathway coupled to a second compression element that is disposed at a second extremity of the garment;
an inflation system coupled to the first air pathway and the second air pathway, wherein the inflation system is configured to force air through the first air pathway and the second air pathway to inflate the first compression element and the second compression element respectively.

2. The system of claim 1, wherein the first air pathway comprises:
a first layer of thermoplastic material bonded to the garment; and
a second layer of thermoplastic material bonded to the first layer of thermoplastic material along two edges thereof to form an inflatable channel therebetween.

3. The system of claim 2, wherein the garment comprises a shirt, wherein the first extremity is the end of a right sleeve of the shirt, and wherein the second extremity is the end of a left sleeve of the shirt.

4. The system of claim 3, wherein the inflation system comprises an electromechanical pump.

5. The system of claim 1, wherein the inflation system comprises a bladder coupled to the shirt, the bladder having an air intake configured to obtain air from the ambient environment, the bladder configured to direct air through a one-way valve into the first air pathway and the second air pathway responsive to compression of the bladder.

6. The system of claim 3, further comprising:
a first pressure sensor coupled to the first compression element;
a second pressure sensor coupled to the second compression element; and
a control system coupled to the inflation system, wherein the control system comprises a microprocessor configured to activate the inflation system to inflate the first compression element and the second compression element to a desired pressure as measured by the first pressure sensor and the second pressure sensor.

7. The system of claim 6, wherein the first compression element comprises:
a stretchable inner bladder layer extending entirely around the right sleeve of the shirt;
a stretchable outer bladder layer coupled to the stretchable inner bladder layer on at least two sides thereof to form an inflatable region therein; and
a non-stretchable outer barrier layer disposed atop the outer bladder layer to limit expansion of the first compression element in the outward direction.

8. The system of claim 7, wherein the stretchable outer bladder layer is coupled to the stretchable inner bladder layer in a series of bladder connection joints disposed about the circumference of the right sleeve of the shirt.

9. An integrated garment and blood flow restriction (BFR) training system, the system comprising:
a garment configured as leggings and comprising an elastic fabric;
a first air pathway coupled to a first compression element that is disposed circumferentially around a right leg portion of the leggings and proximate the crotch seam;
a second air pathway coupled to a second compression element that is disposed circumferentially around a left leg portion of the leggings and proximate the crotch seam;
an inflation system coupled to the first air pathway and the second air pathway, wherein the inflation system is configured to force air through the first air pathway and the second air pathway to inflate the first compression element and the second compression element respectively, wherein the inflation system comprises a bladder disposed in the sole of a shoe and coupled to the first air pathway and the second air pathway via a one-way valve, the bladder having an air intake configured to obtain air from the ambient environment, and the bladder configured to direct air through the one-way valve into the first air pathway and the second air pathway responsive to compression of the sole of the shoe resulting from a wearer walking,
wherein the first compression element is configured with a release valve that vents pressure from the first compression element above a specified pressure to maintain the first compression element at or below the specified pressure.
